SEQUENCE LISTING (28 sequences)

SEQ ID NO: 1 - DNA, 3132 bp, Mus musculus
gagttccgcgcagcgctccggagctgaaaggactgcaatcagcgcccgaggtcaggaggaataggtggcgcctagagagcgggtggcaccagagttgcggcagcccggaggtggaagctctcgagaggggcagaacagcctgcagggagcagcggggcgccggcaggctggtgtggggggagaccctgcaggagtcatggccgggctacgagccaggcggggccctgggcgcaggctactggtgttgtccacgctgggtttttgcctaatgctgcaagtcagtgccaagagacctcctaagacgcccccctgcccacccagctgctcctgcaccagggacaccgccttctgcgtggactctaagtcggtgcccaagaacctgccttcagaggtcatctccctgacgctggtgaatgctgccttctcagagatccaggatggagccttctcccatctgccgttgttgcagttcttgttactcaactctaacaagttcacactgattggagacaatgccttcataggactgtcgcacctgcagtacctcttcatagagaacaatgacatctgggctctctccaagtttaccttcagaggactgaagtccttgacacacctttctctggccaacaataacctgcagacactgcccagagacatcttccgtcccctggacatcctgagtgacttagacctccggggcaacgcacttaactgtgactgcaaggtaaagtggctggtggagtggctggcacacaccaacaccacggtggctcccatctactgcgccagcccgccccgcttccaggaacacaaggtgcaggacttgcctcttcgagaattcgactgcatcaccactgattttgtcctctaccagaccctgtccttcccagcagtgtctgcggaacctttcctttactctagtgacctctacttggctctggcccagccaggtgccagcgcctgcaccatcctcaagtgggactatgtggaacgacagcttcgagactatgacagaattccagccccttcagctgtgcactgcaagccgatggtggtggatggccagctctatgtcgttgtggcccagctgtttggtggctcatacatttaccactgggaccccaacaccacacgtttcaccaagttgcaggacatcgacccacagcgcgtgcgcaaacccaatgacctggaagccttccgcatcgacggtgactggtttttcgcagtggctgacagctccaaggcaggtgccaccagcctctaccgttggcaccagaatggcttctattcccaccaggccctgcacgcctggcaccgcgacaccgacctggagtttgtggacggtgagggcaaaccacggttaattgtgtctagcagctcgcaagcacctgtcatctatcaatggagtcgttctcagaaacagtttgtggctcagggagaggtgactcaggtacctgatgcccaggccgtgaaacactttcgtgctggccgagacagctacctgtgccttagccgctacattggcgactccaagatcctgcgctgggaaggtacccgtttctctgaggtgcaagcccttccctcccgaggctcgctggccctacagcccttcctggtgggtggtcaccgctacctggccctgggcagtgacttctccttcacccagatctaccagtgggatgaggggcgacagaaatttgtacggttccaagagctggcagtacaggcaccccgggccttctgctacatgcctgctggagatgcccagctgctcctggcccccagtttcaagggacaaacactggtgtaccgacatgttgtggtggaccttagtgcctagaggggggctaagtcctttgggttcctcagggtggcactgtacgatgggtggggacctctgtgagcaacttgtggccccaagtgaagtcacaggtggccagcctgtctatgtagctacacacacatacacaaagaagtgggcctgggcacacaccagggactagcccaggggagccattcccattactgtaataagcacctacgtatgcaccaggcacccagtgtccaggctctctatgtgcgcaccaacagcccctgatcttcagtgttccctgctctgctccgttgctagcgtgtctgagtaacaaagcgagtgcaggaaggggtgagccacagccttctcttctcagcttagctttctgctctctggcctctggatgcccgaggtgcttgtgtacctgtttggcatccacagttgcagcccccctgcaggtgtgcccacacttccaagtgtgcctgcccacgccatgctccttctgcatgcacctgtggacactgatggacaaactcagctgcatacttgtgcataccccaccacacttctgattctgtatctgcacacgtctgggggtgtgtacgtgagcggactaggtggctgggcaaaccggcgtccttgataccccgtagctgctcttcttggagggtgcctacagcacccacactttctctgctctccccagtgtgccccctgtgtggctcataaggatagcaacagaaaacgtgaccatggcctcgtggggacaaagccacttcatccagaagcaataataacaagaggcagcagcaactgggttcttcacagtgcagtatagctctcctcactagagttttcttccacgactcagtcccagctcaaagggacacgggtgggaaggctttctctggatagctattgctttttcaagctcgggccatctcctctctcaccctatctttgagatgcggatcccaaagtgtgattgctccttgactggatatataaaaaaaaatcagccaaggcaagaatcctgagacagcctgagctacttgtgtgggcgccaggaacatgttggtcgccctggcacccctccatgccatggagatgccccccggaggcacagctcggtggaggctggatgaggatggcagggagggacacctgggaaccaggcctctgctgccctctgctgtcctctgggagcagcagccctgaagtaggaaatgatcaggctgagaggatttcttgcggcctgatgcacctggggataatgctaagattcctttgcttcgaataaagttccaccccactgc

SEQ ID NO: 2 - DNA, 3604 bp, Mus musculus
accatgaagtcacgggtctcgtccatggccacttgctcaggcacaagtgcaatgggatgcgtattgctgaggagctggtggctgtggcccgtgaccggggctcccatgacaacattacagtcatggtggtcttccttagggagcccctggagctgctagagggtggagtccaggggacaggggatgcccaggcagatgtaggaagccaggacttgtccactggcctctcagaacttgagatcagtaatacctcacagagaagctaagtggtccaggcctccaggccccatccggactctccctctgccctcgtgatcctccccttcaaagaccttagatccaaaaggtatagtgggcaggggtgcacactctcacagcattcccttagcaccccagcccttcatgttgcctaccacagcctgtcctcatggctgcagaactgcagtaggcagctagtggatctcagaaggaaacataggaaaatggcctcaccgaagagcagatggctgaggtgatcagggccggacaggtctgggaacagaaaccacaggcacctgctggtagcctaaccaaaaacacaggtcaggtcttggaggaccctcacacagatcctggaatgagatctgcagccaggtgtccagcccaggcttgtggcttctcattgtacccaaggctgggagggtttggtctgtactaacacacaagctcgcagtcctgcttgactgctggcttcccaaagaggagacattggtcttgctgggaggcacagcaggagagtgacccactgccactgcactctaactgagtactaaggccactagggctttctagacctcgctttccccttgagcttcctggggaggtgaagtgaggtgtgtgtgtgtgtgtgtgtctttgtgtgcttagatttattgcagggaaaggtctaatccagaatcagtattcaggctttgtcatgttgtatcagtgccaaggtgaccctcaaggtcatgtaacttaagcaaagcttagcatttattttattcctgaaaacttaagtattttacttttttgtgtgttcgtggagacatttgcagtattaatgattttatttttcctaaatcgggatggaaacaaacttttccaggttatgttaataagccacttaagtgccttaaacagctttggtgtagatgagaattgctgggtccgtcatggatcctgataggcagacgtgttgaggttctcaaagaggctgcatttgttgtgggaaggcacactttcagccagcactctttctgggaaagtccttttgtgcgtctgggcacctcagaacttccttgaagtgatgggtaaaggctgtgactatgggcacctggggtgacactggcctccttgtccaccttggagtctggctggttcccagctgagagaaccttactgctcttggctagaagattgacacactcaatctctcttgaaactctcttaagtgtgtttgcagttatgttgattgagttgttgaagacagtgcttaggtgaggaacttgggcaagcccagcagtccaccatctttctcctgtgacttgtgtcctaacctgctagactctgtcctgacctgagcagcatgaaagacctccacatccctagagcagtgatggatacagtcaggcaaggaaaccaagtgagggttgggggggataccaagaatagaaggactaggaggccctcaacaagggccaagcgagtcaaggctgctctctgcagacactggcaggtccagagaataatggtggcttagcagaggctcagggaaggatcagtaggaagaagggtggctgtgaggtaggtccccatgaaggagacatagttcacctgtagagtgaaagccctagcaagacttgcagagctttgttcattgcacctaagccctctttgaaaagaagcaagggagctcatacttattcctctgaggcaagttcacttccagcagtaggagagcgagccagccaaatgcagctgggcacccacctgtatgcttgccatttacccattcccacagcagggatggaagagctggggccatgtagttgattccttgatcaagtctcctgataggaacgtgatcactcagatggctgcccacctaatagctaggtattgcttaacttgaaggcctgctgccttccgggggggggggggggtgtcagaatccagcatcctcagtgccatctcaacacagggtgtggttgttggctccagcctgcctgtagcagtgaccttcatagagggagataaattgattttgtttttctcccatttttgctaacctgatcattgctggtaagagtggaatttgggcctccttggtggtctggattcaaactcgcatgtaaagatgtgccaaatatcaaggcctcagtgtaaagggaagcccctttcccaggcttgtctgagtgcctcccccacccttccccctgtctggcaatggtctgggaccaacatcaggcactgctccatcctgatctagtggccaaggaagaccacattctcctactgttgagggctggccttgagcgagttcctaggtacagagaactctttttgtctggactggagttgccagtaccaagcttacaagacttgttggccacctcctcctcagagtctcttcccaaaagaatcccctagccaggataatgggtggcttagtagaggctcagggaaggaacagggagaagggtggctgtgagctcaagtccccctctttgccaccatcacattgtgtgagcggaaccagtcactgcacctctcaggcctcagggaaaggcatggagggcatgcatagcattaccaggagctgccagagcacgggagcccatcctccaccagcagcagagaggggttaagtattattagtgtctattcttaaaattaagtgggctggaacagagctgagttacaggcgccagtgtcttacacagatacaaaagctgaagccgtcagcctgcctttgcaggtacagctgcctcccaaccaaggcctggaggcgttaagaggtgggagggcacaagcaggctttcaggagtgttgaagcaagggatctgcagcatagctttgggcgtctccagaactcatggtgagggaggcaccaacacctagaggtcttaaaagccctgcattcaccatgatctgtctgcccctgcccttttgggttcttctgcatgcagtgcccccctctcctgatgctaagttttggcatgggtgtcctggctacctcaactgtattctcaaggccaggaggatggctctggccatacttacggaagccaggccaatttgtgtgaagcaagctgggcttctggtttgattcagcttctgtagcctccagggaccatcctctggagtcttgtgctttcactggactcttcagcttcaccaaacacaccaagctttgccatttacatgccatgtgggccttgccaagggttatttcaggggtcttcctgggagtcttgttctaagacaccgtgtttctttagtcttagggacaactcccaaggtgatgagctcataggacgtcttcccatggggccctgtactggtgtcaataaaccatatattc

SEQ ID NO: 3 - DNA, 3683 bp, Mus musculus
gtttccggcttgaggatccgtcgcgtttctgaggcgacgcctcagtcagcagttaggatccgctcggagcggtcggtctcggccccggcgtcaccatgggggccgtcctcggcgtcttctccctcgccagctgggtcccgtgcctctgtagtggtgcatcatgtctgctgtgcagttgctgtcccatcagtaagaattccactgtaactcggctcatctacgcttttatcctcttccttggcactattgtgtcttgcatcatgatgacagaaggcatacaaactcaactgaagaagattcctggattctgtgaaggaggatttcaaatcaagatggttgatacaaaggcagagaaagattgtgacgtgctggtcggttttaaagctgtgtatcggatcaactttgctgtggccatcattttctttgccttctttttgctcatgttaaaagttaaaacaagtaaagatcccagagcagcagtgcacaacgggttttggttcttcaaaatcgctgccattattggtatcatgattggatctttctacatccctgggggcagttttactgaagtctggtttgttgctggaatgttgggggcctctttcttcattatcatccagctggtgctcttggtagacatggctcactcttggaatgaattatgggtaaatcgaatggaggaaggaaacccaaggctctggtatgctgccttgctgtcctttacaagcctcttttacatcctctccatcgtctttgctgcgctgctctacgtcttctacaccaagcctgacgactgcacagaaaacaaggtcttcatcagcctcaacctgattttttgtgttgcagtttctattgtgtccatcctccctaaagttcaggaacatcagcctcgctctggcctcctgcagtcctccatcatcactctgtacaccctttacctcacgtggtcagccatgaccaatgaacctgagcggtcctgcaatccctcccttatgagcatcatcacacacttaacttcaccaactgtgtctcctgcaaattcaactactcttgctcctgcctatgctccgccgtcacagagtgggcactttatgaatttggatgatatttggggactgattatctttgttttctgccttatatattctagcttccgtacttcgagcaacagccaagttaacaagctgaccctctctgggagtgacagtgttatccttggtgataccaccaatggagccaatgatgaagaggatggacagccacggagggctgtagacaatgagaaggagggggtgcagtatagctactcctttttccacttgatgctctgctgtgcctccttgtacatcatgatgaccataaccagctggtacagccctgatgccaaattccagaaggtatccagcaagtggctagctgtgtggttcaaaatgggctccagctggttgtgcctcctcctttacctctggactcttgtggctcccctggtcctcacaggtcgggacttcagctgagctcagtgtgtcaaggacactgataaagctgaccagagtctccttttctgaaaatgcatatccattttgcgtttcatcaacgagactattaagtgaacgctttgcagatttggctgtattcaggtttatatcaaaaggcaagattgagtaatgcttgatgcagaatctgagctttcatatatatatatatatatatatatatatacacacacacacacacacatatatatgtttatttgtaaggctatagcacaaagggaacatttttgtgttttaacatgaactacagctgtgctgtgaagagaattctttataaagacctgtagattcctacaactttggtttaagttttaagttagaagattgttggatatttaaggctattttatatttctattacagtctccttaaaaaccaaaaaggaatgcattaatccacatttcccttcttcagaggtgtaatgtcctggctcttggcaaggaattatgtattttaggtcagtccccagaaatgcagctgctcatacagctgagagaaggctattattgagttcctttacttactttttatactacactgatgctgcttgatagaagtctgtggctttgtcagatatgtcacccaagtaaatgctttgtagatctgattaaaatgaaaagctcacttgagaaacactgcagagttatgtaatgatcttgttgtgagtgtgagaaagtcaaaggcatgtcagtttattacatttgcaacataaaagtacttaattaaaatagatatctagtttcttgtttgtttgtgtatttgtttagaaagggtcttttccacataacccagacttgcttggaatgcagatcatcctatctccacttccaagtactgaggaggaggtttctatcaccatagctagtcttttcttaaagatccttctgcagtggggttgataggttttcttttattgttgttttcttgtttagagagtcccaaatcaatctgacatttcaggcaaaatgctcctcttctaacacttaagatttgactagtcaagtttttaagttcatttagaaactaccctaaatattttctctggggaagatcaaagtaggtaagaaacagtttaggcactgtcaaaaaagatattcctgaaaagcagtgtgcaaaggaggtatgataaaacaggcttttcaagagaaactactatcggttctgcactaggccctgttggggtgaagacaagacagacattcttccatctaaggaaaatgagcggaccagcctggtaaattagtgctgaaaactcataaacagaaatcagtggatagaggctagcacagtgacttcctcgagagagtgctgttaggatttgtaacgtaggttcacaccaggtatcagggcagttcatctgagatttgaactaaagatgtaagggggtttggggaaaaattgacagcttagagaatttccacattggtgtttaaggggataggaaaaacttatttccagtttaatctgtgtacataggttctgcttgggatcctggttgggatggaggtggtcccttcatatgatgaataggtttgggagtcagacctctggggtctgcctgacctctgctattttctaggttgagtggctgtctaatgctcactgagtacatgctgtgcttgtgaccttgagcactctgcctgggcctcatgcagaacttagtaccagagattatttgaagataaagttgtctcagaatggatatgttgtaaggatgtttgacagtaagtgccaatgtcactatggattaaagcaagactaaatgtacttaaaaggtcagaagtgttcagaatctgatagaggctgggttgaatgtagttttagagaattaaacatagttgtaatatagacagctaggtagagctggaggctagggaagctgggtttgatgggttgttggaactactgtcctttaggatcatgtgtcaaagcaaactacaaaggatttgtgaagcagataattgtggcccttaagcttaactgtgtgagacaaatacctgtttttaacagtaggggaaccataaatttcaaagcggatcctattctttgtcttatatgattaaaaataaatggagttctaaactaccaaataatgtaatagagttcaataaagggttttttttttttttc

SEQ ID NO: 4 - DNA, 3519 bp, Mus musculus
ggcactggtcccagagtcccagccagcttccaggagctggggtaggtccaaagccttagctaggcctggggatgaagaagatgaagatcttgctcctccgtaaagaaggatggccagggatggggttgttgggacactgaggtgacttcctgcaaggcaacgctggccctagaatctggacccccctttttttttttcccaggcaggcgcagcgctcgctgcctcccttttccccgccagccaggttgttggcgcgttagacctcccagcctcacggggctgctgtgcggctgcgacgaagctggcgactgcggcgctgcagcagaggcaagggctccagaggcggcgccacgagctggtgattgcatctggaagtgcccatgacagagctggtgtcctccaggggagggtcccctacaggggacggggaggagggtctgggggacgatcaaggcctggttatccaccacccggcggaggaacagtgccaccgctgcccactgtgcgggcagaccttctcccagcagcccagcctggtgcggcaccagaaggcgcacgtgggggctggccgcgcagccgccttcgtgtgtcccgagtgcggcaaggccttcagcgtcaaacacaacctggaggtgcaccagcgcacgcacaccggcgagcggcccttcgcctgccccgagtgcggccgctgcttcagcctcaagcagaatctgctcacgcaccagcgcatccacagcggcgagaagccgcaccagtgcgcgcagtgcggtcgctgcttccgcgagccgcgcttcctgctcaaccaccagcgcacccacgcgcgcatgcccacgccgcacccgcgccgtcccggtgtcttcggggagcggcgaccctacttctgccctcgctgcggcaagagcttcgcgcgtgagggttcgctcaagacccaccagcgcagccatggccacgggcccgagagccaagcggcccatttgagccgtgtgctatgacctgtgggagacctgctaccaccagctgtctccaatccagaagccacatccaagggtgctaaattgcagtgccctgtctggatgggctcctgggagggggcagggccagcttagggtgttgaaaggcctccgggccacccctcccccttgtcttcctgcccttggaccccttggctggctgcatgtatctgctttgggcacctgcattaatttgcctcctccggctgcctagagcagcatgtcagacttgaaagctacttggagaaggtcacttgccacggttcccagacccaagctcggtccccaaagcctcagcctcctgtacccactgcctggtggccttgagaagccattccactccatgtctcagtttgcttatctatacgaagccaacaggacctcatggcccagaacctgcagggcgcatctgggcatggaaactaaaactggagtctcgtaggtcctgggttcctgacctgaagacacgtctcctcagaagtcctacctctggaggtgccctgtgttggtagagcatgggattagaggctgattccatgccttggtgggaagactccccagatctgacttgtcacagctgggcaagcctcatttggcttcctgatcctagacctcagctcgggggtggaggcagacatcacctgtgtggagaagtggcttctctcctccagcctggctcagcccttggttgaagggcaaggacaaggtgtgactgttgtgtaccgttgcagcaagttgatggcagactgggctcaagaggcaaaacttccctgatgacatggagagcacgtgaaggcttccatgcccgttcccagctagacaggtgactgcttcaggggcttgtggccctttaagtggaggctgtggacctcgtcctagaactcgcgtactcatggaactctcagagtgccaagcagtttcaggagcctctggactccagcctaaagacctcagccctggggagtccatcttccatcctttccctactccacacaagtgtgacgctctcctggtctcctcttgtttgggaataaagaattctgaggctgagccccttttagtccatccaatccctttctcttcaatctgacagcaccccaaatttctggagccaggcttccctccccagacactgcccaagactgactcctgggcttggttccttttccctgttttgtgtcggacaggatgtagggttttacagtggagccctctgcttgcctttggctctaacttccctgtggagtctcaagggaaggttggcctgctgaagggatggtaaataattttctctgtgtctgttcctcgggaaaggaatcgtacttccactagccatatgcaaacaattggacctcctagctgggcaggcttgcagctccaggcaggtatcacctgggaaggtgggggttgcagggcaccagaaccctgcacatggtgggtctaagcagtgggaaaagccagtggaggagttgcatgtgtctcctaagtaagcaggggtacaacttggagatgagggtcttgggccactgctggtcaaggactcatatccttacatattagcccgtcagtcgagagcccacaactgtctccctcccttcctagaatgagccactcattaggaaagggggaggggctttcttttctatcaggacttggtagaaacctctggattcctgacttggggtcttactgcctgcattccccatgcagtttgggttacaagttcctactgaacccctatgccttattctggggtcactgagtactcaaagagatcagagaaagggaaggcgctacccataccatgggaacttccatgcaccctcttttctaacttagggcgactttcaccatgcctctcagctaactgcccagcttgtctcatccccactgtaactgagccatgccttcccttgtcctgctctgaaatgttcttcccctagcctctaaacccacttattcaccagggccaagcctctcagcctacccagcccatgttccttaacagtcctcgtgttaatacatggtgtctccctgagtatcatgtgctgtttaatcttgtgcttgtttcttctgtgtttgtgtatgtatacgtgctgtgtgtgcccgtgtgcaccttgagtccccaactagactgtgagctccctgagggcaaggggctgtcatttcttcttcaacttctagtgggtctagcccaagaacctctcagaagacaactggatgaaacacatgctggaggtcatctgaggtggagggttgtgtgcatggggagagtccacatggcttagcaagaggtctatgctgttgggctatggcctgaccccttggatcaacaatggatgctcaaggtctcacatccttgcaaattggaggtagtagaaacaggagggacaccccctcccccaacacagatagaccatggctacaccggagtgcacatgaatggttggatattcccaaaggtccctgatatctgttggaggaggtgactcaaataaatgttctctac

SEQ ID NO: 5 - DNA, 1995 bp, Mus musculus
gagttcctgtttcggcccctgggccttctagacgcgtccgtaactgttcccgctgggtctgccttgccacccaggatggggtcccacccgcgcttgggccgtcccttcttggggatacaaggcgcctcggcccctccttgaccatctgcgcttgtggtgtaactgctctcaggatgaagggcggcgagggggacacgggcgaacaggccccgctgaacccggaggtggacagccccgcgggctcagccacgtaccgggagttcgtgcaccgcggctacctggaccttatgggggccagtcagcactctctgcgggcgctcagctggcgccgcctctacctcagccgggctaagctcaaagcctccagccgcacatctgctctgctgtcgggcttcgccatgcagtggccccgggaagtggaactgaagattccagagtgtggcctgcagatgtgtcctggttcctccctacttagggccagctgtgcggccccaagtcgacatccactccagttgccatctttaggaagttgtgacatcatagaaggtggccatggtggaggtacagctggagaacgatcatgaatacccaccaggcctgctggtggcatttagtgcctgcaccactgtgttagtagctgtgcacctctttgcactgatggtctccacatgtctgctgccccatattgaagccgtgagcaacatccacaatctcaactctgtccaccagtcaccacaccaacgactgcacagatacgtggagcttgcctggggcttctctactgccttgggtacctttctcttcctggctgaagttgttctggtgggctgggtcaagtttgtgcccattggggcacccatgggtaaaccagctcctgttgtacctatgtcccaggtgccacctgtgactgtctcccttagtttagcttctaacctcacaccatcctctgcttctattaccacatcacaacagccttccaaagcctgtccaccccggcaagtgtgtgatagtgctcatggaccaggctggcaggcagctatggcctccacggcaatcatggtacctgtgggactagtgtttatggcctttgccctacatttctaccgatccttggttgcccacaagacagaccgccacaagcaggagctggaggagctcagtcgcctgcagggggagctgcaggctgtgtaagcctggctttagactgttgcaagcattgcctcagaaaccccagggatcgacagacctccttccttgccctaggctggagccacctcctgtaagctctgaggtagaggccaagtacctgcccagcagcatcccacactcccagggatgatccctgtcatcatgttctggtgggttttgtaaattctgctctgcttggggaactgtttcagcctaaggatcctgggttaaatgagaggaaagaggcccgcctgccttagtgagatccctggtggtgctgagcagttgtttgctcctccaggaaggtgacatgtctgcacaactgtcctcaaggcaatcagcccttgcttatctgtataatctgctttgcagttggcatcctgggagagattttacagggctcctcagatgagccacttcacccttggtgacttgtggtggtcgttccactgccctaggcactgactgtctggccagaactgggagaggaggaagtattgacgagtgggagattgtgcagctacttgacaagggttggtgagttggtaagggagaagattcagcccttagaatgcagaactcttcttcagacactgattttttttcttttgttttgtttttcaagccagagtttcactatgtctgttgctctggctgtcctggaacactctctgtagaccaggctggccttgagctcagagatgtacctgcctctgcctcccatgtacagggattaaaggcatgcaccaccaccacctgacactgagtttttgatgcaccttgttttgtaaataaagtgtgtctcac

SEQ ID NO: 6 - DNA, 1842 bp, Mus musculus
gacttccggtcagcgaggcctcttcctccgccgctttgccgggtctccctgcccactgcccctcgggtcttcccggagtggcgccggcagctccgtgcgtgtctctccggaagcccgcgctcgctgctcgcctcgccgcaccgcttcgccgaggctcgcgagccgggaggggctgggccctgccgcgcgcgtcgaggcccccgcagctccgttgaggccaccgcgctgccgggatgccgcggccggggtcggcgccgcgctgggcggccgccgcgggccgttggggtctctactgctgctcctgccggctccgagcggcggctccgagatcaccttcgagctgcccgataatgccaagcagtgcttctacgaggacatcactcagggcaccaagtgcaccctggagttccaggtgattaccggtggtcactacgatgttgattgtcgattggaagatcctgatggtaaagtgttgtacaaggagatgaagaaacagtatgatagtttcaccttcacagcctccagaaatgggacgtacaagttttgcttcagcaatgagttttctactttcacacacaaaacagtgtatttcgatttccaagttggagaagacccacctttgtttcccagtgagaaccgagtcagtgcgcttacccagatggaatccgcctgcgtgtccattcatgaagctctcaagtccgtcattgactatcagacacatttccggctccgggaagctcaagcgcgaagccgagcagaggatctgaatacaagagtggcctattggtcggtgggggaagccctcatcctctgcgtggttagcgtagggcaggtgttcttctgaagagctttttctcagataaaagaacaccacaacccgcgtcggatcctaactacgcttcgagatgcgccattgccaccgcaatgttactctcctcagttagggactgaagaacttaacattggcagcattctaaaaccctactcatacttgttgggaaagaggtacagtgcatatccccatggggagaggacaccttttttatttgtaaaggtggaaaattttggaccttaatgcagctattcatatgaaatacctaacaccaatgatctactttttcttggtttatatgtactcttcctacactaacctttggtattcagattccttttagccatttaaaagtccttttcttaataggtagttgatactttaaaataaaaacttaggtttattgtctgtgtatattacagaggaagaaaactgccttttaattgtttatagtgaataaagttgtgtttttaagaaaaccaaatgtgattaactgtagccaagccctattctgcactgcttaattttacgggggaaaataatctaccatagaacttttagtttaatagtgacacaattttcattgctatatcatgatataatttatttcttgctagtttgaaaatgtacagttttttgggttgttttttttctccattctatgatttataactagctatattatctgtttaaaaaactgttaggataaagaataatatgagacactatggtatggtaagtaaaataggaacatttttagtgagttctctgtcactgcattcctgggcttttataaagaagagaggacctgagtcctgtgagcattctggtgaactagttcttggttgagccattgccagcgtaggtcagcagttgtgagctcgtgtcacataagtgccatatgtcttatcctcacagtgtaatgcactgcgttcatttttatctctctccattttgtcttgtttctgcagtctaattagcagctctacaattaaacatttagaatgctg

SEQ ID NO: 7 - DNA, 1856 bp, Homo sapiens
gctcgggtgggggggcgccgagtgggggtggcggccagcatgctgctcggctgcggctcggcctcccacacccgcggctccctagtcctcgccagcagcggcggcggcagagcagcgagcggcgcccgcgtctgcagcggcgccgggtccgagcgcgcggcgcggcggtgggggtcggggcccgggcggggagcggggaccgggcatggcgctgcggagaggcggctgcggagcgctcgggctgctgctgctgctgctgggcgccgcgtgcctgataccgcggagcgcgcaggtgaggcggctggcgcgctgccccgccacttgcagctgtaccaaggagtctatcatctgcgtgggctcttcctgggtgcccaggatcgtgccgggcgacatcagctccctgagcctggtaaatgggacgttttcagaaatcaaggaccgaatgttttcccatctgccttctctgcagctgctattgctgaattctaactcattcacgatcatccgggatgatgcttttgctggactttttcatcttgaatacctgttcattgaagggaacaaaatagaaaccatttcaagaaatgcctttcgtggcctccgtgacctgactcacctttctttggccaataaccacataaaagcactaccaagggatgtcttcagtgatttagactctctgattgaactagatttgaggggtaataaatttgaatgtgactgcaaagccaagtggctatacctgtggttgaagatgacaaattccaccgtttctgatgtgctgtgtattggtccaccagagtatcaggaaaagaagctaaatgacgtgaccagctttgactatgaatgcacaactacagattttgttgttcatcagactttaccctaccagtcggtttcagtggatacgttcaactccaagaacgatgtgtacgtggccatcgcgcagcccagcatggagaactgcatggtgctggagtgggaccacattgaaatgaatttccggagctatgacaacattacaggtcagtccatcgtgggctgtaaggccattctcatcgatgatcaggtctttgtggtggtagcccagctcttcggtggctctcacatttacaaatacgacgagagttggaccaaatttgtcaaattccaagacatagaggtctctcgcatttccaagcccaatgacatcgagctgtttcagatcgacgacgagacgttctttgtcatcgcagacagctcaaaggctggtctgtccacagtttataaatggaacagcaaaggattctattcttaccagtcactgcacgagtggttcagggacacggatgcggagtttgttgatatcgatggaaaatcgcatctcatcctgtccagccgctcccaggtccccatcatcctccagtggaataaaagctctaagaagtttgtcccccatggtgacatccccaacatggaggacgtactggctgtgaagagcttccgaatgcaaaataccctctacctttcccttacccgcttcatcggggactcccgggtcatgaggtggaacagtaagcagtttgtggagatccaagctcttccatcccggggggccatgaccctgcagcccttttcttttaaagataatcactacctggccctggggagtgactatacattctctcagatataccagtgggataaagagaagcagctattcaaaaagtttaaggagatttatgtgcaggcgcctcgttcattcacagctgtctccaccgacaggagagatttcttttttgcatccagtttcaaagggaaaacaaagatttttgaacatataattgttgacttaagtttgtgaaggtgtggtgggt

SEQ ID NO: 8 - DNA, 601 bp, Homo sapiens
accaggaagttgttggcctggtccagagccacctgaccaggcagcagggcagcgggctccgtgtcgccgaggagctggtggctgcggcccgggagcggggctcccacgacaacatcacggtcatggtggtcttcctcagggacccccaagagctgctggagggcgggaaccagggagaaggggacccccaggcagaagggaggaggcaggacttgccctccagccttccagaacctgagacccaggctccaccaagaagctaggtggtttccaggcccctgccctccccttcctcccatccttgtccttctctccctcagaagcctcaggacccaacaggtggcaggcagtggacagggtgcccgccccacagtgctttccccagcaccccagagccagtcgggacaccccccgcagcccgtcctggtggctgtggaactgcactgggtggcgggcagatggtggaaggcagcttaggagacctcaccaaagagaagatggaccggctcttgctcccagctcctattaggcccggggtgggaccagaggtcataggtgcccaacggcagccaaaccaaagacactggtgtgcatggggcag

SEQ ID NO: 9 - DNA, 1776 bp, Homo sapiens
ggcgtcaccatgggggctgtgctgggtgtcttctccctcgccagctgggttccatgcctctgcagcggtgcctcatgtttgctgtgtagttgctgtcctaacagtaagaattccacggtgactcgcctcatttatgctttcattctcctcctgagcactgtcgtatcctatatcatgcagagaaaagagatggaaacttacttgaagaagattcctggattttgtgaagggggatttaaaatccatgaggctgatataaatgcagataaagattgtgatgtgctggttggttataaagctgtgtatcggatcagctttgccatggccatctttttctttgtcttttctctgctcatgttcaaagtaaaaacaagtaaagatctccgagcggcagtacacaatgggttttggttcttcaaaattgctgcccttattggaatcatggttggctctttctacatccctgggggctatttcagctcagtctggtttgttgttggcatgataggggccgccctcttcatcctcattcagctggtgctgctggtagattttgctcattcttggaatgaatcatgggtaaatcgaatggaagaaggaaacccaaggttgtggtatgctgctttactgtctttcacaagcgccttttatatcctgtcaatcatctgtgtcgggctgctctatacatattacaccaaaccagatggctgcacagaaaacaagttcttcatcagtattaacctgatcctttgcgttgtggcttctattatatcgatccacccaaaaattcaggaacaccagcctcgctccggcctcttgcagtcctccctcatcaccctctacactatgtacctcacctggtcagccatgtccaatgaacctgatcgttcctgcaatcccaacctgatgagctttattacacgcataactgcaccaaccctggctcctggaaattcaactgctgtggtccctacccctactccaccatcaaagagtgggtctttactggattcagataattttattggactgtttgtctttgttctctgcctcttgtattctagcatccgcacttccactaatagccaagtagacaagctgaccctgtcagggagtgacagcgtcatccttggtgatacaactaccagtggtgccagtgatgaagaagatggacagcctcggcgggctgtggacaacgagaaagagggagtgcagtatagctactccttattccacctcatgctctgcttggcttccttgtacatcatgatgaccctgaccagctggtacagccctgatgcaaagtttcagagcatgaccagcaagtggccagctgtgtgggtcaagatcagctccagctgggtctgcctcctgctttacgtctggacccttgtggctccacttgtcctcaccagtcgggacttcagctgaacctctgagtgccaaggacaccactggaactcacaaaggtctccttcaccgaaaacccatataccttttaagtttgtttcaactaaaatattaagtgaatgctttgcaagtttgactgtatgcaggtttatatcagaaggtgagattgaataatgcttgatgcagaatcgaaacttctcatttatctgtatattatgtttacttctaaggatatagcacaaagggaacattttttgtttaaagtgaactacagctgtgctgtgaagagagttctttataaagcctgtaggttcttttaactttggtttaaaatgtaagataggaaaatgttggatatttgaggcc

SEQ ID NO: 10 - DNA, 1287 bp, Homo sapiens
  unknown (965): n is a, g, c, t
atggggccgcccccgggcttgggccggcccggctggggttcccgaggcgcttccgccccgtagtgaccgcctggtgccgccccccccccaggatgaagggcggcgagggggacgcgggcgagcaggccccgctgaaccctgagggcgagagccctgcaggctcggccacgtaccgggagttcgtgcaccgcggctacctggacctcatgggggccagtcagcactcgctgcgggcgctcagctggcgccgcctctacctcagccgggccaagctcaaagcttccagccgcacgtctgccttgctctcgggcttcgccatgcagtggcctcacaggaagtgggatggagggaggtcccagggtgaggatcagcagacacagtcctggctcctccctgcttgtggccagctgtgtgaccctggacaggcctccaccccgcttgcaagccagtgggaaactgagatcatggagggtggccatggtggaggtgcagctggagagtgaccacgagtacccaccaggcctgctggtggccttcagtgcctgcaccaccgtgctggtggctgtgcacctctttgcactcatggtctccacgtgtctgctgccccacattgaagctgtgagcaacatccacaacctcaactctgtccaccagtcgccacaccagagactgcaccgctacgtggagctggcctggggcttctccactgccctgggcacctttctcttccttgctgaagttgtcctggttggttgggtcaagtttgtgcccattggggctcccttggacacaccgacccccatggtgcccacatcccgggtgcccgggactctggcaccagtggctacctcccttagtccagcttccaatctcccacggtcctctgcgtctgcagcaccgtcccaggctgagccagcctgcccaccccggcaagcctgtggtggtggtggggcccatgggccagnctggcaagcagccatggcctccacagccatcatggtacccgtggggctcgtgtttgtggcctttgccctgcatttctaccgctccttggtggcacacaagacagaccgctacaagcaggaactagaggaactgaatcgcctgcagggggagctgcaggctgtgtgagactggtgttagccaccgctcactgcaagcactgcctccctccggggtctgtaagaggccgcaggggcctacagacctcatccccccatcccctggctggagccacttccagtggccactctcaggcagagttcagattcctgcccgcagggtcct

SEQ ID NO: 11 - DNA, 848 bp, Homo sapiens
ttgcggggtggcctgcgctgaggcctgccgcgcgcggtgagtccgcgcagacctgaccctgcgtctcgcagctcggttgaggccgccgccgccttctcgggatgccgcggccggggtccgcgcagcgctgggcggccgtcgcgggccgttgggggtgcaggctgctcgcactgctgctactggtgcctggacccggcggcgcctctgagatcaccttcgagcttcctgacaacgccaagcagtgcttctacgaggacatcgctcagggcaccaagtgcaccctggagttccaggtgattactggtggtcactatgatgtagattgtcgattagaagatcctgatggtaaagtgttatacaaagagatgaagaaacagtatgatagttttaccttcacagcctccaaaaatgggacatacaaattttgcttcagcaatgaattttctactttcacacataaaactgtatattttgattttcaagttggagaagacccacctttgtttcctagtgagaaccgagtcagtgctcttacccagatggaatctgcctgtgtttcaattcacgaagctctgaagtctgtcatcgattatcagactcatttccgtttaagagaagctcaaggccgaagccgagcagaggatctaaatacaagagtggcctattggtcagtaggagaagccctcattcttctggtggttagcatagggcaggtatttcttttgaaaagctttttctcagataaaagaaccaccacaactcgtgttggatcataactacgttttgagaattgatgcaccattgccactgtaatattgctgtcctctaattaattttaggtactgaag

SEQ ID NO: 12 - protein, 548 aa, Mus musculus
MAGLRARRGPGRRLLVLSTLGFCLMLQVSAKRPPKTPPCPPSCSCTRDTAFCVDSKSVPKNLPSEVISLTLVNAAFSEIQDGAFSHLPLLQFLLLNSNKFTLIGDNAFIGLSHLQYLFIENNDIWALSKFTFRGLKSLTHLSLANNNLQTLPRDIFRPLDILSDLDLRGNALNCDCKVKWLVEWLAHTNTTVAPIYCASPPRFQEHKVQDLPLREFDCITTDFVLYQTLSFPAVSAEPFLYSSDLYLALAQPGASACTILKWDYVERQLRDYDRIPAPSAVHCKPMVVDGQLYVVVAQLFGGSYIYHWDPNTTRFTKLQDIDPQRVRKPNDLEAFRIDGDWFFAVADSSKAGATSLYRWHQNGFYSHQALHAWHRDTDLEFVDGEGKPRLIVSSSSQAPVIYQWSRSQKQFVAQGEVTQVPDAQAVKHFRAGRDSYLCLSRYIGDSKILRWEGTRFSEVQALPSRGSLALQPFLVGGHRYLALGSDFSFTQIYQWDEGRQKFVRFQELAVQAPRAFCYMPAGDAQLLLAPSFKGQTLVYRHVVVDLSA

SEQ ID NO: 13 - protein, 69 aa, Mus musculus
MRIAEELVAVARDRGSHDNITVMVVFLREPLELLEGGVQGTGDAQADVGSQDLSTGLSELEISNTSQRS

SEQ ID NO: 14 - protein, 472 aa, Mus musculus
MGAVLGVFSLASWVPCLCSGASCLLCSCCPISKNSTVTRLIYAFILFLGTIVSCIMMTEGIQTQLKKIPGFCEGGFQIKMVDTKAEKDCDVLVGFKAVYRINFAVAIIFFAFFLLMLKVKTSKDPRAAVHNGFWFFKIAAIIGIMIGSFYIPGGSFTEVWFVAGMLGASFFIIIQLVLLVDMAHSWNELWVNRMEEGNPRLWYAALLSFTSLFYILSIVFAALLYVFYTKPDDCTENKVFISLNLIFCVAVSIVSILPKVQEHQPRSGLLQSSIITLYTLYLTWSAMTNEPERSCNPSLMSIITHLTSPTVSPANSTTLAPAYAPPSQSGHFMNLDDIWGLIIFVFCLIYSSFRTSSNSQVNKLTLSGSDSVILGDTTNGANDEEDGQPRRAVDNEKEGVQYSYSFFHLMLCCASLYIMMTITSWYSPDAKFQKVSSKWLAVWFKMGSSWLCLLLYLWTLVAPLVLTGRDFS

SEQ ID NO: 15 - protein, 203 aa, Mus musculus
MTELVSSRGGSPTGDGEEGLGDDQGLVIHHPAEEQCHRCPLCGQTFSQQPSLVRHQKAHVGAGRAAAFVCPECGKAFSVKHNLEVHQRTHTGERPFACPECGRCFSLKQNLLTHQRIHSGEKPHQCAQCGRCFREPRFLLNHQRTHARMPTPHPRRPGVFGERRPYFCPRCGKSFAREGSLKTHQRSHGHGPESQAAHLSRVL

SEQ ID NO: 16 - protein, 212 aa, Mus musculus
MVEVQLENDHEYPPGLLVAFSACTTVLVAVHLFALMVSTCLLPHIEAVSNIHNLNSVHQSPHQRLHRYVELAWGFSTALGTFLFLAEVVLVGWVKFVPIGAPMGKPAPVVPMSQVPPVTVSLSLASNLTPSSASITTSQQPSKACPPRQVCDSAHGPGWQAAMASTAIMVPVGLVFMAFALHFYRSLVAHKTDRHKQELEELSRLQGELQAV

SEQ ID NO: 17 - protein, 119 aa, Mus musculus
MKKQYDSFTFTASRNGTYKFCFSNEFSTFTHKTVYFDFQVGEDPPLFPSENRVSALTQMESACVSIHEALKSVIDYQTHFRLREAQARSRAEDLNTRVAYWSVGEALILLVVSVGQCFF

SEQ ID NO: 18 - protein, 545 aa, Homo sapiens
MALRRGGCGALGLLLLLLGAACLIPRSAQVRRLARCPATCSCTKESIICVGSSWVPRIVPGDISSLSLVNGTFSEIKDRMFSHLPSLQLLLLNSNSFTIIRDDAFAGLFHLEYLFIEGNKIETISRNAFRGLRDLTHLSLANNHIKALPRDVFSDLDSLIELDLRGNKFECDCKAKWLYLWLKMTNSTVSDVLCIGPPEYQEKKLNDVTSFDYECTTTDFVVHQTLPYQSVSVDTFNSKNDVYVAIAQPSMENCMVLEWDHIEMNFRSYDNITGQSIVGCKAILIDDQVFVVVAQLFGGSHIYKYDESWTKFVKFQDIEVSRISKPNDIELFQIDDETFFVIADSSKAGLSTVYKWNSKGFYSYQSLHEWFRDTDAEFVDIDGKSHLILSSRSQVPIILQWNKSSKKFVPHGDIPNMEDVLAVKSFRMQNTLYLSLTRFIGDSRVMRWNSKQFVEIQALPSRGAMTLQPFSFKDNHYLALGSDYTFSQIYQWDKEKQLFKKFKEIYVQAPRSFTAVSTDRRDFFFASSFKGKTKIFEHIIVDLSL

SEQ ID NO: 19 - protein, 68 aa, Homo sapiens
LRVAEELVAAARERGSHDNITVMVVFLRDPQELLEGGNQGEGDPQAEGRRQDLPSSLPEPETQAPPRS

SEQ ID NO: 20 - protein, 473 aa, Homo sapiens
MGAVLGVFSLASWVPCLCSGASCLLCSCCPNSKNSTVTRLIYAFILLLSTVVSYIMQRKEMETYLKKIPGFCEGGFKIHEADINADKDCDVLVGYKAVYRISFAMAIFFFVFSLLMFKVKTSKDLRAAVHNGFWFFKIAALIGIMVGSFYIPGGYFSSVWFVVGMIGAALFILIQLVLLVDFAHSWNESWVNRMEEGNPRLWYAALLSFTSAFYILSIICVGLLYTYYTKPDGCTENKFFISINLILCVVASIISIHPKIQEHQPRSGLLQSSLITLYTMYLTWSAMSNEPDRSCNPNLMSFITRITAPTLAPGNSTAVVPTPTPPSKSGSLLDSDNFIGLFVFVLCLLYSSIRTSTNSQVDKLTLSGSDSVILGDTTTSGASDEEDGQPRRAVDNEKEGVQYSYSLFHLMLCLASLYIMMTLTSWYSPDAKFQSMTSKWPAVWVKISSSWVCLLLYVWTLVAPLVLTSRDFS

SEQ ID NO: 21 - protein, 217 aa, Homo sapiens
  unknown (163): Xaa = any amino acid
MVEVQLESDHEYPPGLLVAFSACTTVLVAVHLFALMVSTCLLPHIEAVSNIHNLNSVHQSPHQRLHRYVELAWGFSTALGTFLFLAEVVLVGWVKFVPIGAPLDTPTPMVPTSRVPGTLAPVATSLSPASNLPRSSASAAPSQAEPACPPRQACGGGGAHGPXWQAAMASTAIMVPVGLVFVAFALHFYRSLVAHKTDRYKQELEELNRLQGELQAV

SEQ ID NO: 22 - protein, 136 aa, Homo sapiens
MKKQYDSFTFTASKNGTYKFCFSNEFSTFTHKTVYFDFQVGEDPPLFPSENRVSALTQMESACVSIHEALKSVIDYQTHFRLREAQGRSRAEDLNTRVAYWSVGEALILLVVSIGQVFLLKSFFSDKRTTTTRVGS

SEQ ID NO: 23 - DNA, 44 bp, Artificial Sequence
  Description of Artificial Sequence NX primer used for constructions of libraries derived from kidney and lungs
gagagagagagcggccgcaactcgagttttttttttttttttvn

SEQ ID NO: 24 - DNA, 43 bp, Artificial Sequence
  Description of Artificial Sequence BS primer used for constructions of libraries derived from stomach, tongue, ES cells and liver
gagagagagaaggatccaagagctcttttttttttttttttvn

SEQ ID NO: 25 - DNA, 51 bp, Artificial Sequence
  Description of Artificial Sequence 2nd primer -adapter with restriction sites Bam HI and Sst I
gagagagagaaggatccaagagctcaattaattaattaaaccccccccccc

SEQ ID NO: 26 - DNA, 45 bp, Artificial Sequence
  Description of Artificial Sequence 2nd primer -adapter with restriction sites Xho I and C13
gagagagagattctcgagttaattaaattaatccccccccccccc

SEQ ID NO: 27 - DNA, 100 bp, Mus musculus
aaaagctggagctccaccgcggtggcggccgctctagaactagtggatcccccgggctgcaggaattcgatatcaagcttatcgataccgtcgacctcga

SEQ ID NO: 28 - DNA, 74 bp, Mus musculus
aaaagctggagctatggcccttatggccgagctcgcggccgcgaattcctcgagggccgatttggccaatcgag

(19) United States
(12) Patent Application Publication
(10) Pub. No.: US 2004/0019006 A1
(43) Pub. Date: Jan. 29, 2004

(54) Novel genes relating to pain and use of the genes for pharmaceuticals

(76) Inventors: Yoshihide Hayashizaki, Ibaraki (JP); Mamoru Kamiya, Tokyo (JP); Tomohiko Suzuki, Kanagawa (JP); Hiroe Hirokawa, Kanagawa (JP); Kaoru Nakao, Kanagawa (JP); Toshiaki Tanaka, Kanagawa (JP)

Correspondence Address: BIRCH STEWART KOLASCH & BIRCH, PO BOX 747, FALLS CHURCH, VA, 22040-0747, US

(21) Appl. No.: 10/434,156

(22) Filed: May 9, 2003

Related U.S. Application Data
(60) Provisional application No. 60/378,955, filed on May 10, 2002.

(51) Int. Cl. A61K 48/00; C12Q 1/68; C07H 21/04; C12P 21/02; C12N 5/06; C07K 14/705

(52) U.S. Cl. 514/44; 435/6; 435/320.1; 435/325; 530/350; 536/23.5; 435/69.1

(57) ABSTRACT

An object of the present invention is to find novel genes that relate to, but are not limited to, pain, such as neuropathic pain, shingles pain, and post-herpetic neuralgia, and are induced upon pain, and provide a reagent, method and pharmaceutical for diagnosing and assessing pain, or preventing and treating pain.

The present invention provides polynucleotides having nucleotide sequences composing novel genes, polypeptides encoded by the polynucleotides, and partial peptides thereof, recombinant vectors containing the polynucleotides, transformants having the recombinant vectors, antibodies against the polypeptides or partial peptides thereof, and the like, which are utilizable in biology, medicine, veterinary medicine, and the like. Further, the present invention provides a method for producing the polypeptides, compounds whose target molecules are the polynucleotides or the polypeptides, and pharmaceuticals containing the polypeptides or the partial peptides thereof, compounds whose target molecules are the polynucleotides or the polypeptides, or antibodies against the polypeptides. They are used particularly to diagnose and assess pain, or prevent and treat pain. Further, the present invention provides a method for identifying compounds and the like using these polynucleotides, polypeptides, or the partial peptides thereof, or salts thereof as a target molecule to prevent and treat pain, a method for using the identified compound to prevent and treat pain, a method for diagnostic monitoring a patient to be subjected to clinical evaluation for pain treatment, a method for monitoring the clinical efficiency of the compound, and a method for identifying a patient having diathesis of pain.

This application claims the benefit under 35 U.S.C. §119(e) of U.S. provisional patent application Serial No. 60/378,955, filed on May 10, 2002, which is incorporated herein, by reference, in its entirety.

### FIELD OF THE INVENTION

The present invention relates to a method and a composition for treating and diagnosing pain, which relates to, but not limited to, neuropathic pain, shingles pain, post-herpetic neuralgia, and the like. More specifically, the present invention relates to novel genes which is identified as being expressed in a pain state differentially from the normal state, or gene products thereof and the like. Further, the present invention relates to a use of the identified novel gene or the gene products thereof for diagnosis and evaluation, and to a use for prophylactic and therapeutic applications or a use as a target for therapeutic intervention. More particularly, the present invention relates to a method for identifying a compound for preventing, treating and diagnosing pain, and a compound found by the identification method, a diagnostic method of monitoring patients to be clinically evaluated upon pain treatment, a method for monitoring the clinical efficiency of a compound, and a method for identifying a patient with diathesis of pain.

### BACKGROUND OF THE INVENTION

The nervous system that transmits pain is a system for knowing the state of the outside or the inside of the body, and it plays an important role as a defense system of the living body. Pain due to exogenous stimulation is useful to protect the body from risk, and pain due to organic disease of the body is an alarm signal to indicate that an abnormality is occurring in the body. However, pain is an unpleasant feeling, and is not beneficial for the person who feels the pain. When the pain lingers, it can be complicated with other functional disorders, and can even cause adverse effects on the treatment. Pathophysiological pain, such as cancer pain and post-herpetic neuralgia, can be itself a serious invasion for the living body. Thus, surgical and medical measures are now actively taken to remove pain unnecessary for the living body. Known measures to prevent and treat pain include nerve block therapy, electric stimulation therapy, surgical therapy, physical therapy, and drug therapy using analgesic agents such as long-known opioids and nonsteroidal anti-inflammatory drugs. However, nerve block therapy and surgical therapy are themselves associated with invasion, and in frequent cases, sufficient analgesic effects cannot be obtained by administration of the analgesic agents. Thus, further improvement of methods for removing pain and research and development of new analgesic agents are still underway.

The International Association for the Study of Pain defines pain as “a sensation or emotional experience which leads to substantial or potential damage of tissues, or a term expressed representing such damage.” That is, when a patient says they have a pain, a pain exists. However, the facts that pain is characteristic in being a subjective feeling and that pain varies depending on patients' reactions have made it difficult to assess and diagnose pain itself. The VAS (Visual Analogue Pain Scale) method and the face scale method, in which the degrees of pain are expressed by pictures of faces, are known and generally used widely as objective methods for assessing pain. However, molecular biological and biochemical analysis methods for monitoring pain have not yet been developed.

Pain can be categorized by origin into nociceptive pain, neuropathic pain, and psychogenic pain. Nociceptive pain is classified into somatic pain and visceral pain, which are respectively caused by activated nociceptors in superficial tissues, such as skin and mucosa, and deep tissues, such as muscle, bone and joint, or activated nociceptor in viscera. In contrast to somatic pain, which is a focal pain, the location and characteristics of visceral pain are often unclear. Psychogenic pain is seen in the case of pain that is anatomically and neurologically unexplainable. Hence, it is thought that mental factors are deeply involved in psychogenic pain.

Neuropathic pain is a pain syndrome, which is caused by neural damage, dysfunction and the like. Typical diseases of neuropathic pain are (1) diseases due to damaged peripheral nerves, including post-herpetic neuralgia, delayed post-operative pain, diabetic neuropathy, post-radiation irradiation neuropathy, protracted pain after blood collection and protracted pain after insertion of an indwelling needle, pain after dismemberment, Complex Regional Pain Syndrome (CRPS), and a part of cancer pain, and (2) diseases due to damaged central nerve system, including thalamic pain, pain after spinal cord injury, trigeminal pain, glossopharyngeal neuralgia, and the like. Patients with neuropathic pain commonly develop symptoms such as spontaneous pain, hyperalgesia (symptom to feel light painful stimuli as strong painful stimuli), allodynia (symptom in which pain is induced by contact stimuli, such as light touching, that normally induce no pain) or the like.

In recent years, research on the developmental mechanisms of pathophysiological pain and research on the pharmacological mechanisms of analgesic agents have revealed that complex neural networks (such as the peripheral tissue, peripheral nerves, spinal cord, brainstem, cerebrum, and the like), many molecules for neural transmission (such as neuropeptides, excitatory amino acids, and their receptors, and the like), transcriptional activation of stress genes, promotion of the synthesis of physiologically active substances, and the like are involved in pain. As a result, therapies against pain have been certainly improved. However, even today, treatment of chronic pain, such as neuropathic pain, is still often difficult. The reasons for this are thought to be not only that patients having chronic pain cannot be simply classified according to their pathological conditions, but also that the onset and maintenance mechanisms as well as the molecules involved in the pathological conditions of pain such as neuropathic pain and the like remainunclear. Hence, elucidation of these mechanisms and related molecules, and development of new pharmaceuticals and procedures to prevent or treat pain are expected.

Since inflammation occurs when tissues are damaged by diseases or external injuries, and pain is occurred, many researchers have studied pain in relation to the expression of factors relating to inflammatory mediators, such as bradykinin, prostanoids, histamine, serotonin, and the like in the peripheral and central nervous systems. Further, since these mediators are induced by cytokines, the relationship of pain with the expression of cytokines has also been reported. For example, it is known that when inflammation is lingered, newly expression of bradykinin B1 receptor (Br. J. Pharmacol., 110: 1141, 1993) or increased expression levels of bradykinin B2 receptor on nociceptors result in enhanced sensitivity of bradykinin, the dolorogenic (pain-causing) substance, and thus pain thresholds decrease (Eur. J. Pharmacol., 429; 161, 2001, Annu. Rev. Pharmacol. Toxicol., 42; 553, 2002). Further, it is known that the expression of cyclooxygenase, the biosynthesis enzyme of prostanoids, is increased by interleukin-1β, the inflammatory cytokine (Nature, 410; 471, 2001).

Regarding studies on pain and gene expression, there are many reports on genes other than inflammatory mediators and cytokines. Enhanced expression of Nerve Growth Factor (NGF) (Br. J. Pharmacol., 115: 1265, 1995) and Brain derived-Neurotrophic Factor (BDNF) (Proc. Natl. Acad. Sci. USA, 96; 9385, 1999), which are of a group called neurotrophic factors, and increased expression of neuropeptides existing in primary sensory neurons, such as substance P, preprotachykinin, and calcitonin gene related peptide have been reported (Eur. J. Neurosci., 10; 2388, 1998, Brain Res., 464; 31, 1988, Pain, 78; 13, 1998). Furthermore, changes in the gene expression of receptors and channels, such as ion channel-type ATP receptor P2X (Br. J. Anesth., 84; 476, 2000), sodium channel (Pain, Suppl6; S133, 1999), potassium channel (Proc. Natl. Acad. Sci. USA, 98; 13373, 2001), and heat-sensitive non-specific cation channel-type vaniloid receptor VR1 (Eur. J. Neurosci., 13; 2105, 2001) have also been reported.

These studies have aimed at specific genes or the products thereof predicted to be involved in pain, and examined changes in expression levels and the like in order to clarify their roles upon occurrence of pain. However, such approaches cannot be used to identify a series of genes or the gene products thereof involved in the onset or the course of the maintenance of the disease. Still more, identification of genes or the products thereof that can function as a target for diagnosing, assessing and treating various pains is impossible.

Aside from the above research, studies to analyze the entire genomic sequences of many organisms, such as mammals including human and mouse, bacteria, and plants have been completed or are underway. However, even if the entire nucleotide sequence of a genome is decoded, it is not easy to find out at which regions functioning genes, which are said to account for only 1.5% of the genome, are present. Hence, studies to analyze the full-length cDNA nucleotide sequences have been conducted. As a result, for the mouse, analytical results concerning approximately 21,000 cDNAs have been reported (Nature, 409; 685, 2001). Further, many nucleotide sequences and amino acid sequences of cDNAs are being registered daily in databases such as GenBank. That is, such information is being accumulated at a rapid pace. However, for many of these registered genes, while their sequences have been analyzed, their functions remain unknown. Accordingly, many researchers are attempting to elucidate these unknown functions of the genes by conducting functional motif searches using sequence information and by examining when and where the genes function, specifically, when and where the genes are expressed.

A few specific genes can be examined by the Northern blotting method and RT-PCR method, which are long known as methods for analyzing expression levels of genes. Recently, various techniques with which the expression of a large number of genes can be thoroughly examined and unknown genes can be identified have been developed. For example, the subtractive hybridazation method, EST method, differential display method, SAGE method, and microarray method have become widely used.

Some attempts to identify genes involved in pain by these techniques have been reported. Kim et al. reported that when they searched by the differential display method for genes with varied expression in the dorsal root ganglion of rats for which neuropathy had been artificially induced, the expression of 4 genes, including heat shock protein 27, apolipoprotein D and the like, were increased, and the expression of 6 genes, including Kv1.2 channels and the like were decreased; (Neuroreport, 12; 3401, 2001). Two of these reported genes were thought to be unknown, novel genes. Further, Yang et al. revealed that when they induced inflammation in one of the hind legs of rats, and searched by the subtraction method for genes showing changes in the expression in the dorsal horn of the spinal cord, some genes containing cystatin C and two unknown genes showed changes in their expression levels (Neuroscience, 103; 493, 2001). These pain-related unknown genes obtained in these studies had no match when their sequences were compared with sequences registered in the GenBank database using BLAST algorithm. Thus these genes are thought to be genes having novel nucleotide sequences. However, to be precise, the nucleotide sequences of these unknown genes are merely sequences of gene fragments. The full-length nucleotide sequences of the genes encoding proteins having physiological functions, namely, the nucleotide sequences of the full-length cDNAs, were not yet shown. Further it is completely unknown that functional proteins, which are the gene products thereof and involved in pain, have what kind of amino acid sequences.

### SUMMARY OF THE INVENTION

An object of the present invention is to find novel genes that relate to, but are not limited to, pain such as neuropathic pain, shingles pain, and post-herpetic neuralgia, and are induced upon pain. That is, an object of the present invention is to provide polynucleotides having novel nucleotide sequences that are utilizable in biology, medicine, veterinary medicine and the like, and the polypeptides and the partial peptides or salts thereof; a method for producing a recombinant vector and a transformant containing the polynucleotide, and the polypeptide; a compound whose target molecule is the polynucleotide or the polypeptide; an antibody against the polypeptide; a compound whose target molecule is the polypeptide or a partial peptide thereof, or the polynucleotide or the polypeptide; and a pharmaceutical containing an antibody against the polypeptide. In particular, an object of the present invention is to use the polynucleotide, the polypeptide or a partial peptide thereof, or a compound whose target molecule is the polynucleotide or the polypeptide, and an antibody against the polypeptide for diagnosing and assessing pain or for preventing and treating pain. Further, an object of the present invention is to provide a method for identifying a compound and the like using as a target molecule for preventing and treating pain the polynucleotide, polypeptide or a partial peptide thereof, or a salt thereof, a method for using the thus identified compound for preventing and treating pain, a diagnostic method for monitoring patients to be clinically evaluated upon pain treatment, a method for monitoring the clinical efficiency of a compound, and a method for identifying patients who have diathesis of pain.

As a result of thorough studies to solve the above problems, we have succeeded in finding and obtaining a full-length cDNA (gene) having a novel nucleotide sequence that is expressed in the pain state differentially from the normal state. We have also succeeded in identifying the human homologues of the novel genes. That is, the present invention provides, for example:

(1) isolated polynucleotides which contain the nucleotide sequences of genes represented by SEQ ID NOS: 1 to 11 or fragments of the genes,

(2) polypeptides which contain amino acid sequences identical to, or substantially identical to amino acid sequences represented by SEQ ID NOS: 12 to 22, and polypeptides which contain amino acid sequences encoded by genes or fragments of the genes having the polynucleotide sequences of (1) above or sequences complementary thereto, or salts thereof,

(3) a partial peptide of the polypeptide of (2) above or a salt thereof,

(4) a method for producing the polypeptide of (2) above or the partial peptide of (3) above or salts of these peptides, which comprises culturing transformants transformed with a recombinant vector containing the polynucleotide of (1) above or a polynucleotide encoding the polypeptide of (2) above, or the partial peptide of (3) above, and allowing the polypeptide or the partial peptide to be generated;

(5) an antibody against the polypeptide of (2) above, or the partial peptide of (3) above, or salts of these peptides;

(6) a transgenic animal, wherein the polynucleotide of (1) above is an expression transgene contained in the genome of the animal, or the expression of the genomic sequence containing the gene encoding the polypeptide of (2) above is hindered or suppressed, or promoted;

(7) a method for screening for a compound or the like that suppresses or enhances the expression of the gene contained in the polynucleotide of (1) above, or for a salt thereof, or a kit for screening for the compound or a salt thereof;

(8) a method for screening for a compound or the like or a salt thereof that promotes or inhibits the activity of the polypeptide of (2) above or the partial peptide of (3) above or a salt thereof, or a kit for screening for the compound or a salt thereof, which uses the polypeptide of (2) above or the partial peptide of (3) above or a salt thereof, or a transformant transformed with a recombinant vector containing the polynucleotide of (1) above or a polynucleotide encoding the polypeptide of (2) above or the partial peptide of (3) above;

(9) a compound or a salt thereof that is obtainable by the screening method or the screening kit of (7) or (8) above, which suppresses or enhances the expression of the gene contained in the polynucleotide of (1) above, or a compound or a salt thereof that is obtainable by the screening method or the screening kit of (7) or (8) above, which promotes or inhibits the activity of the polypeptide of (2) above or the partial peptide of (3) above, or a salt thereof;

(10) a pharmaceutical, which comprises the polypeptide of (2) above or the partial peptide of (3) above or a salt thereof, or the antibody of (5) above or the compound of (9) above or a salt thereof,

(11) a method for diagnosing patients, which comprises measuring the polypeptide of (2) above or the partial peptide of (3) above in the samples of the patients, or measuring the differential expression of the gene containing the nucleotide sequence of (1) above, or a method for monitoring the therapeutic effects caused by the pharmaceutical of (10) above;

(12) a diagnostic agent, which comprises the antibody of (5) above;

(13) the pharmaceutical of (10) above, which is a therapeutic and prophylactic agent against pain; and

(14) a method for treating and preventing pain, which comprises administering the polypeptide of (2) above or the partial peptide of (3) above or a salt thereof, or the antibody of (5) above or the compound of (9) above or a salt thereof.

The present invention further provides, for example:

(15) the polypeptides of (2) above or salts thereof, which have amino acid sequences that are substantially identical to the amino acid sequences represented by SEQ ID NOS: 12 to 22, and respectively have approximately 50% or more (preferably approximately 80% or more, particularly preferably approximately 90% or more, and most preferably approximately 95% or more) homology with the amino acid sequences represented by SEQ ID NOS: 12 to 22;

(16) the polypeptides of (2) above or salts thereof, which have amino acid sequences that are substantially identical to the amino acid sequences represented by SEQ ID NOS: 12 to 22, by deletion of 1 or 2 or more (preferably, approximately 1 to 30) amino acids, addition of 1 or 2 or more (preferably, approximately 1 to 30) amino acids, substitution of 1 or 2 or more (preferably, approximately 1 to 30) amino acids with (an)other amino acids, or a combination of these;

(17) a recombinant vector, which contains a polynucleotide encoding the polypeptide of (2) above or the partial peptide of (3) above; and

(18) a transformant, which is transformed with the recombinant vector of (17) above.

The present invention enables identification and characterization of a target useful for prognosis, diagnosis, monitoring and rational drug design for the symptoms of pain, and/or therapeutic intervention. We naturally recognize that the present invention can be applied, for example, not only for basic research on, such as molecular weight markers, tissue markers, chromosome mapping, identification of genetic disorders, and designing of primers and probes, but also certainly for the prevention and treatment of pain, neuranagenesis, treatment for spinal cord injury, treatment for neurodegeneration, gene therapy, regulation of differentiation and proliferation of neurons or glia cells.

### DETAILED DESCRIPTION OF THE INVENITON

“Differential expression” used in this specification refers to temporal and/or quantitative and qualitative differences in tissue expression patterns of genes. A differentially expressed gene can be “a target gene” of the present invention. Further, a differentially expressed gene or a gene product thereof can be “a target molecule” of the present invention. “A target gene” used in this specification refers to a differentially expressed gene which relates to pain in such a way that the regulation of the expression level of the gene or the gene product thereof, or the regulation of the activity of the gene product acts to improve the state of pain. A compound which regulates the expression of a target gene or a gene product thereof or a compound which regulates the activity of the product of the target gene can be used to treat pain. The genes according to the present invention are differentially expressed in association with pain, and the products thereof can interact with products of genes which play crucial roles in the mechanism of pain. However, these genes may also be involved in other crucial processes in the mechanism to cause pain. “Homology” used for the amino acid sequences and nucleotide sequences in this specification, can be determined, for example, using BLAST algorithm (for example, using default setting) described in Altschul et al., J. Mol. Biol., 215; 403, 1990. Program for conducting BLAST analysis is available from the National Center for Biotechnology Information (http://www.ncbi.nlm.gov/).

Obtainment of the Polynucleotide of the Present Invention

Any pain-related animal model can be used to identify a gene that is differentially expressed in association with pain. Total RNA or mRNA may be isolated from the spinal dorsal root ganglion, spinal cord, brain and the like obtained from these model animals, which correspond the pain transmission pathway, used as experimental materials. Such RNA samples can be purified using any non-selective RNA isolation method. Furthermore, a large number of cell samples can be easily treated using a method known by a person skilled in the art, for example, the one-step RNA isolation method (Chomcznski, U.S. Pat. No. 4,843,155). RNA, which is contained in the collected RNA sample and is produced by a gene expressed differentially, for example, specifically in the state of pain, can be identified by various methods known by a person skilled in the art.

For example, genes whose expression levels fluctuate can be selectively identified using specific (differential) screening (Proc. Natl. Acad. Sci. USA, 85; 208, 1988), deletion hybridization (Nature, 308; 149, 1984, Proc. Natl. Acad. Sci. USA, 88; 2825, 1984) and specific display (Science, 257; 967, 1992), or a SAGE method (Science, 276; 1268, 1997), or preferably using a subtractive hybridization method, such as a suppression subtractive hybridization method (Proc. Natl. Acad. Sci. USA, 93; 6025, 1996). The subtractive hybridization technology generally involves isolation of mRNA from two different types of sources, for example, a control tissue and a test tissue, hybridization between the respective isolated mRNAs or between single-stranded cDNAs reverse-transcribed from the isolated mRNAs, and removal of all the hybridized double-stranded nucleic acids. In this method, the remaining, unhybridized mRNA or single-stranded cDNA may be a clone derived from a gene differentially expressed between the two types of mRNA sources. Next, such a single-stranded cDNA is used as a starter substance to construct a library containing a clone that is induced from a gene to be differentially expressed.

Various methods can be used to determine the characteristics of an identified gene. First, using the nucleotide sequence of an identified gene that is sequenced by a standard method known by a person skilled in the art, homology of the sequence with one or a plurality of known sequence motifs that provide information on the biological functions of the identified gene product is shown. Second, the distribution of mRNA produced by the identified gene in a tissue and/or cell type is analyzed using a standard method known by a person skilled in the art. Examples of such a method include the Northern, RNase protection and RT-PCR analyses. Further, using the standard in situ hybridization method, information about which cells of a given tissue or cell population express the identified gene is obtained. Third, the gene can be positioned on a genetic map by a standard method using the identified gene sequence. Fourth, the biological functions of the identified gene can be directly evaluated using appropriate in vitro and in vivo systems. An example of the in vivo system is, but not limited to, a transgenic animal system. Alternatively, the function of the activity of the identified gene product may be evaluated by increasing or decreasing the activity levels in in vitro system and/or in vivo system. Information obtained by such analyses can suggest appropriate methods for treating or regulating pain and the like.

The polynucleotide of the present invention may be any polynucleotide, as long as it contains the nucleotide sequence of a gene, which can be obtained by the above method and is differentially expressed in the state of pain (target gene). Examples of cells and tissues from which the differentially-expressed gene is derived include cells of a human or a warm-blooded animal (for example, guinea pigs, rats, mice, chickens, rabbits, pigs, sheep, cattle and monkeys) including an embryo, wherein the cells include, for example, hepatocytes, splenocytes, neurons, glia cells, pancreas β cells, bone marrow cells, mesangial cells, Langerhans cells, epidermal cells, epithelial cells, endothelial cells, fibroblasts, fibrous cells, muscle cells, adipocytes, immunocytes (for example, macrophages, T cells, B cells, natural killer cells, mast cells, neutrophils, basophils, eosinophils, monocytes, and dendritic cells), megakaryocytes, synoviocytes, chondrocytes, osteocytes, osteoblasts, osteoclasts, mammary glandular cells or interstitial cells, or precursor cells of these cells, stem cells or cancer cells; and every tissue in which these cells are present, such as the brain, each site of the brain (for example, bulbus olfactorius, amygdaloid nucleus, bulbus base of the cerebrum, hippocampus, thalamus, hypothalamus, cerebral cortex, medulla oblongata, and cerebella), the spinal cord, the dorsal spinal root, hypophysis, stomach, pancreas, kidney, liver, gonad, thyroid gland, gallbladder, bone marrow, adrenal gland, skin, muscle, lung, gastrointestinal tract (for example, large intestine, and small intestine), blood vessel, heart, thymus, spleen, salivary gland, peripheral blood, prostate gland, testis, ovary, placenta, uterus, bone, cartilage, joint, and skeletal muscle. Further, the above gene derived from the above cell or tissue may be genomic DNA, cDNA, or synthetic DNA. Vectors used for libraries may be any of bacteriophages, plasmids, cosmids, phagemids, and the like. Further, total RNA or mRNA fractions prepared from the above cells or tissues may be used to be directly amplified by Reverse Transcriptase Polymerase Chain Reaction (hereinafter, abbreviated as “RT-PCR method”). The polynucleotides of the present invention may be any polynucleotides, as long as the polynucleotides contain the nucleotide sequences represented by SEQ ID NOS: 1 to 11, or have nucleotide sequences that hybridize under stringent conditions with the nucleotide sequences represented by SEQ ID NOS: 1 to 11, and encode polypeptides having characteristics substantially the same as those encoded by the polynucleotides of the present invention. “Substantially the same” indicates that these characteristics are qualitatively the same, but quantitative factors, such as the degree of the characteristics, and the molecular weight of a polypeptide may differ. As a polynucleotide which can hybridize under stringent conditions with the polynucleotide having any one of the nucleotide sequences of SEQ ID NOS: 1 to 11, for example, a polynucleotide containing a nucleotide sequence having approximately 60% or more, preferably approximately 70% or more, and more preferably approximately 80% or more homology with any one of the nucleotide sequences of SEQ ID NOS: 1 to 11 is used. Hybridization can be performed according to a method known per se or a method according thereto, for example, a method described in Molecular Cloning 2nd Edition (J. Sambrook et al., Cold Spring Harbor Lab. Press, 1989) or the like. In addition, when commercially available libraries are used, hybridization can be performed according to the method described in the instructions attached thereto. More preferably, hybridization can be performed under stringent conditions. Stringent conditions indicate, for example, conditions including a sodium concentration of approximately 19 to 40 mM, and preferably, approximately 19 to 20 mM, and a temperature of approximately 50 to 70° C., and preferably, approximately 60 to 65° C. More specifically, as a probe for hybridization, for example, polynucleotides having the nucleotide sequences represented by SEQ ID NOS: 1 to 11 or the complementary sequences thereof, or fragments of the polynucleotides are used.

As the polynucleotide of the present invention, a synthetic oligonucleotide, single-stranded DNA, double-stranded DNA, RNA or the like can be used.

The polynucleotide of the present invention can be isolated by amplifying nucleic acids using PCR method using synthetic primers having partial nucleotide sequences of the polynucleotide of the present invention. Alternatively, the polynucleotide of the present invention can be isolated by screening a genomic DNA library incorporated in an appropriate vector by hybridization using as a probe the labeled or unlabeled polynucleotide of the present invention or a fragment thereof. For example, hybridization can be performed according to a method described in Molecular Cloning 2nd Edition (J. Sambrook et al., Cold Spring Harbor Lab. Press, 1989) or the like. When commercially available libraries are used, hybridization can be performed according to the method described in the instructions attached thereto. Primers used to perform the PCR method are not specifically limited, as long as they have partial nucleotide sequences of the polynucleotides of the present invention, and can be appropriately designed based on the nucleotide sequence information of the polynucleotide of the present invention. Further in the PCR method, PCR reaction conditions that enable optimization of the yield and specificity of PCR amplification products, and production of an amplification product with a length that can be resolved by conventional gel electrophoresis technology can be selected. Such reaction conditions are known by a person skilled in the art. Examples of important parameters in the reaction include the primer lengths and the nucleotide sequences, and the temperatures and times for annealing and elongation processes.

The isolated polynucleotide may be used intact depending on the purpose, or may also be used, if desired after, for example, digestion with restriction enzymes, or addition with a linker. The polynucleotide may have the translation initiation codon, ATG on the 5′ terminal side, and may have the translation termination codon, TAA, TGA or TAG on the 3′ terminal side. These translation initiation and termination codons can also be added using an appropriate synthetic DNA adaptor. Further, any appropriate polynucleotide convenient for confirming the production of a polypeptide that is encoded by the polynucleotide, or for purifying the polypeptide, or for preparing a fusion polypeptide can be added at 5′ or 3′ proximal to the translation initiation codon, or at 5′ proximal to the translation termination codon. For example, by using pGEX vector the polypeptide can be expressed as a fusion protein with glutathione-S-transferase (GST). In general, such a fusion protein has many advantages such as its soluble property and enabling easy purification of polypeptides from lysed cells by adsorption to glutathione-agarose beads, followed by elution in the presence of free glutathione.

Production of Recombinant Vector or Recombinant Expression Vector of the Present Invention and Recombinant Host Containing Polynucleotide of the Present Invention

The recombinant vector or the recombinant expression vector of the present invention can be produced by, for example, (i) excising a target DNA fragment from the polynucleotide of the present invention and (ii) ligating the DNA fragment into an appropriate recombinant vector or ligating downstream of a promoter in a recombinant expression vector. As vectors, various commercially available vectors for recombination can be used. For example, plasmids derived from Escherichia coli (for example, pBR322, pBR325, pUC12, and pUC13), plasmids derived from Bacillus subtilis (for example, pUB110, pTP5, and pC194), plasmids derived from yeast (for example, pSH19, and pSH15), bacteriophages, such as λ phage, animal viruses, such as retrovirus, vaccinia virus, and baculovirus, and further, pA1-11, pXT1, pRc/CMV, pRc/RSV, and pcDNAI/Neo are used. Promoters used in the present invention may be any appropriate promoter corresponding to a host to be used for the expression of a gene. When animal cells are used as a host, examples of promoters include SRα promoter, SV40 promoter, HIV-LTR promoter, CMV promoter, and HSV-TK promoter. Of these, CMV (cytomegalovirus) promoter, SRα promoter and the like are preferably used. When a host is a bacterium of the genus Escherichia, trp promoter, lac promoter, recA promoter, λPL promoter, 1pp promoter, T7 promoter and the like are preferred. When a host is a bacterium of the genus Bacillus, SPO1 promoter, SPO2 promoter, penP promoter, and the like, and when a host is yeast, PHO5 promoter, PGK promoters, GAP promoter, ADH promoter and the like are preferred. When hosts are insect cells, polyhedrin promoter, p10 promoter and the like are preferred.

In addition to these promoters, the expression vector contains “an expression-regulating nucleotide regulatory element,” such as an enhancer or an operator, which is known in the art to direct and regulate the expression. The regulatory element is functionally related to the polynucleotide of the present invention in the expression vector. “Functionally related to” means that the regulatory element can also function when the polynucleotide of the present invention is expressed.

In addition to the above examples, a vector containing splicing signal, poly-A addition signal, a selectable marker, SV40 replication origin (hereinafter, may also be abbreviated as “SV40ori”) and the like can be used.

Examples of a selectable marker include dihydrofolate reductase (hereinafter, may also be abbreviated as “dhfr”) gene [methotrexate (MTX) resistance], ampicillin resistance gene (hereinafter may also be abbreviated as “Ampr”), and neomycin resistance gene (hereinafter may also be abbreviated as “Neor,” G418 resistance). Particularly when dhfr gene is used as a selectable marker using dhfr gene-deficient Chinese hamster cells, a recombinant host having a target gene can also be selected using a thymidine-free medium. Further, if necessary, a signal sequence appropriate for a host is added to the N-terminal side of the polypeptide of the present invention.

When a host is a bacterium of the genus Escherichia, PhoA-signal sequence, OmpA-signal sequence and the like, when a host is a bacterium of the genus Bacillus, α-amylase signal sequence, subtilisin-signal sequence and the like, when a host is yeast, MFα-signal sequence, SUC2-signal sequence, and the like, and when a host is an animal cell, insulin-signal sequence, α-interferon signal-sequence, antibody molecule-signal sequence and the like can be used, respectively. Transformants can be produced using the thus constructed vector containing the polynucleotide of the present invention.

Examples of a host to be transformed include bacteria of the genus Escherichia, bacteria of the genus Bacillus, yeast, insect cells, insects, and animal cells. Specific examples of bacteria of the genus Escherichia that are used herein include Escherichia coli K12-DH1 (Proc. Natl. Acad. Sci. USA, 60; 160, 1968), JM103 (Nucleic Acids Res., 9; 309, 1981), JA221 (J. Mol. Biol., 120; 517, 1978), and HB101 (J. Mol. Biol., 41; 459, 1969). Examples of bacteria of the genus Bacillus that are used herein include Bacillus subtilis M1114 (Gene, 24; 255, 1983), and 207-21 (J. Biochem., 95; 87, 1984). Examples of yeast that is used herein include Saccharomyces cerevisiae AH22, NA87-11A, DKD-5D, and 20B-12, Schizosaccharomyces pombe NCYC 1913, and NCYC2036, and Pichia pastoris KM71.

Examples of insect cells that are used herein include, when a virus is AcNPV, an established cell line derived from larva of Mamestra brassicae (Spodoptera frugiperda cell; Sf cells), MG1 cells derived from the mesenteron of Trichoplusia ni, High Five TM cells derived from eggs of Trichoplusia ni, cells derived from Mamestra brassicae, and cells derived from Estigmena acrea. When a virus is BmNPV, an established cell line derived from silkworm (Bombyx mori N cells; BmN cells) or the like is used. For example, as the Sf cells, Sf9 cells (ATCC CRL 1711), and Sf21 cells (for all of the above examples, In Vivo, 13; 213, 1977) are used. For example, as an insect, larva of silkworm and the like are used (Nature, 315; 592, 1985). As animal cells, for example, monkey cells COS-7 (COS7), Vero, Chinese hamster cells CHO (hereinafter, abbreviated as “CHO cells”), dhfr gene-deficient Chinese hamster cells CHO (hereinafter, abbreviated as “CHO (dhfr-) cells”), mouse L cells, mouse AtT-20, mouse myeloma cells, rat GH3, and human FL cells are used.

Bacteria of the genus Escherichia can be transformed according to, for example, a method described in Proc. Natl. Acad. Sci. USA, 69; 2110 (1972), Gene, 17; 107 (1982), or the like. Bacteria of the genus Bacillus can be transformed according to, for example, a method described in Molecular General Genetics, 168; 111 (1979), or the like. Yeast can be transformed according to, for example, a method described in Methods in Enzymol., 194; 182 (1991), Proc. Natl. Acad. Sci. USA, 75; 1929 (1978), or the like. Insect cells or insects can be transformed according to, for example, a method described in Bio/Technology, 6; 47 (1988), or the like. Animal cells can be transformed according to, for example, a method described in Cell Technology, special volume 8, New Cell Technology Experimental Protocols, 263 (1995) (SHUJUNSHA), Virology, 52; 456 (1973), or the like. In this manner, transformants transformed using expression vectors containing the polynucleotide can be obtained.

Obtainment of the Polypeptide of the Present Invention

Polypeptides having amino acid sequences which are identical to or substantially identical to the amino acid sequences represented by SEQ ID NOS: 12 to 22 of the present invention (hereinafter, referred to as “the polypeptide of the present invention”) may be derived from cells [for example, hepatocytes, splenocytes, neurons, glia cells, pancreas β cells, bone marrow cells, mesangial cells, Langerhans cells, epidermal cells, epithelial cells, endothelial cells, fibroblasts, fibrous cells, muscle cells, adipocytes, immunocytes (for example, macrophages, T cells, B cells, natural killer cells, mast cells, neutrophils, basophils, eosinophils, monocytes, and dendritic cells), megakaryocytes, synoviocytes, chondrocytes, osteocytes, osteoblasts, osteoclasts, mammary glandular cells or interstitial cells, or precursor cells of these cells, stem cells or cancer cells] of a human or warm-blooded animals (for example, guinea pigs, rats, mice, chickens, rabbits, pigs, sheep, cattle, and monkeys) including embryos, or all tissues in which these cells are present, such as the brain, each site of the brain (for example, bulbus olfactorius, amygdaloid nucleus, bulbus base of the cerebrum, hippocampus, thalamus, hypothalamus, cerebral cortex, medulla oblongata, cerebella), spinal cord, hypophysis, stomach, pancreas, kidney, liver, gonad, thyroid gland, gallbladder, bone marrow, adrenal gland, skin, muscle, lung, gastrointestinal tract (for example, large intestine, small intestine), blood vessel, heart, thymus, spleen, salivary gland, peripheral blood, prostate gland, testis, ovary, placenta, uterus, bone, cartilage, joint, and skeletal muscle. The polypeptide may also be a recombinant polypeptide or a synthetic polypeptide.

Examples of amino acid sequences which are substantially identical to the amino acid sequences represented by SEQ ID NOS: 12 to 22 include amino acid sequences respectively having approximately 50% or more, preferably approximately 60% or more, further preferably approximately 70% or more, more preferably approximately 80% or more, particularly preferably approximately 90% or more, and most preferably approximately 95% or more homology with the amino acid sequences represented by SEQ ID NOS: 12 to 22. As polypeptides having amino acid sequences substantially identical to the amino acid sequences represented by SEQ ID NOS: 12 to 22 of the present invention, for example, polypeptides having amino acid sequences substantially identical to the above amino acid sequences represented by SEQ ID NOS: 12 to 22 and having characteristics substantially the same as the characteristics of the polypeptides having the amino acid sequences represented by SEQ ID NOS: 12 to 22 are preferred. “Substantially the same characteristics” means that the characteristics are qualitatively the same. Hence, it is preferred that characteristics such as agonist effect and antagonist effect are equivalent (for example, approximately 0.01 to 100-fold, preferably approximately 0.1 to 10-fold, and more preferably 0.5 to 2-fold). However, the degrees of these characteristics and quantitative factors such as the molecular weights of polypeptides may differ. Examples of the polypeptides or salts thereof of the present invention are preferably polypeptides or salts thereof having amino acid sequences that are identical to or substantially identical to the amino acid sequences represented by SEQ ID NOS: 12 to 22.

Examples of the polypeptides of the present invention include proteins and the like containing amino acid sequences derived from the amino acid sequences represented by SEQ ID NOS: 12 to 22 by deletion of one or two or more amino acids (preferably, approximately 1 to 30 amino acids, further preferably, approximately 1 to 10 amino acids, and more preferably, several (1 to 5) amino acids); amino acid sequences derived from the amino acid sequences represented by SEQ ID NOS: 12 to 22 by addition of one or two or more amino acids (preferably, approximately 1 to 30 amino acids, further preferably, approximately 1 to 10 amino acids, and more preferably, several (1 to 5) amino acids); amino acid sequences derived from the amino acid sequences represented by SEQ ID NOS: 12 to 22 by insertion of one or two or more amino acids (preferably, approximately 1 to 30 amino acids, further preferably, approximately 1 to 10 amino acids, and more preferably, several (1 to 5) amino acids); amino acid sequences derived from the amino acid sequences represented by SEQ ID NOS: 12 to 22 by substitution of one or two or more amino acids (preferably approximately 1 to 30 amino acids, further preferably, approximately 1 to 10 amino acids, or more preferably, several (1 to 5) amino acids) with (an)other amino acids; or amino acid sequences derived from the same by a combination of these. When an amino acid sequence contains insertion, deletion or substitution as described above, a position of the insertion, deletion or substitution is not specifically limited.

The polypeptide in this specification has the N-terminus (amino terminus) on the left end and the C-terminus (carboxyl terminus) on the right end, following the general rule for the notation of peptides. The polypeptide of the present invention including polypeptides containing the amino acid sequences represented by SEQ ID NOS: 12 to 22, normally has a carboxyl group (—COOH) or carboxylate (—COO—) at the C-terminus, but the C-terminus may be amide (—CONH₂) or ester (—COOR). As R in ester, for example, in addition to a C1-6 alkyl group such as methyl, ethyl, n-propyl, isopropyl, n-butyl, or the like, a C3-8 cycloalkyl group, such as, cyclopentyl, cyclohexyl, or the like, a C6-12 aryl group, such as phenyl, α-naphthyl, or the like, a phenyl C1-2 alkyl group, such as benzil, phenethyl, or the like, a C7-14 aralkyl group, for example, an α-naphthyl-C1-2 alkyl group, such as α-naphthylmethyl, a pivaloyloxymethyl group which is generally used as ester to be used orally, or the like, is used. When the polypeptide of the present invention has a carboxyl group (or, carboxylate) at positions other than the C-terminus, those with an amidated or esterified carboxyl group are included in the polypeptide of the present invention. An ester that is used in this case is, for example the above ester at the C-terminus. Further, the polypeptide of the present invention also includes a polypeptide wherein an amino group of the amino acid residue (for example, a methionine residue) at the N-terminus is protected by a protecting group (for example, a C1-6 acyl group, such as C1-6 alkanoyl, for example, formyl group, or acetyl group), a polypeptide wherein the glutamine residue at the N-terminus, which is generated by in vivo cleavage, is pyroglutaminated, a polypeptide wherein a substituent (for example, —OH, —SH, amino group, imidazole group, indole group, or guanidino group) on the side chain of an intramolecular amino acid is protected by an appropriate protecting group (for example, a C1-6 acyl group, such as C1-6 alkanoyl group, such as a formyl group, or an acetyl group), and a complex polypeptide or the like, such as a so-called glycopolypeptide to which sugar chains are bound. As a specific example of the polypeptide of the present invention, for example, human-derived polypeptides having the amino acid sequences represented by SEQ ID NOS: 18 to 22 are used.

The polypeptides of the present invention or salts thereof are preferably substantially pure. Herein, “substantially pure” refers to a polypeptide or a salt thereof with purity of 80% or more, preferably 85% or more, more preferably 90% or more, and particularly preferably 95% or more. The purity in this case can be measured by, for example, high performance liquid chromatography, SDS-PAGE, or the like.

Obtainment of Partial Peptide of the Polypeptide of the Present Invention, Polynucleotide Encoding the Partial Peptide, Vector Containing the Polynucleotide, and Recombinant Host Containing the Polynucleotide

Partial peptides of the polypeptides of the present invention (hereinafter, referred to as “the partial peptide(s) of the present invention”) are partial peptides of the polypeptides of the present invention as described above, and preferably may be any peptides as long as they have characteristics substantially the same as the above polypeptides of the present invention. For example, among a constitutive amino acid sequence of the polypeptide of the present invention, a peptide having an amino acid sequence of at least 5 or more, preferably 20 or more, further preferably 30 or more, more preferably 50 or more, and most preferably 80 or more amino acids is used. The meaning of the term “substantially the same characteristics” is as described above. Further, the partial peptide of the present invention may have an amino acid sequence wherein 1 or 2 or more (preferably, approximately 1 to 10, further preferably, several (1 to 5)) amino acids may be deleted, or wherein 1 or 2 or more (preferably, approximately 1 to 20, more preferably, approximately 1 to 10, and further preferably, several (1 to 5)) amino acids may be added, or wherein 1 or 2 or more (preferably, 1 to 20, more preferably, approximately 1 to 10, and further preferably, several (1 to 5)) amino acids may be inserted, or wherein 1 or 2 or more (preferably, approximately 1 to 20, more preferably, approximately 1 to 10, and further preferably, several (1 to 5)) amino acids may be substituted with (an)other amino acids.

The partial peptide of the present invention normally has a carboxyl group (—COOH) or carboxylate (—COO—) at the C-terminus, and the C-terminus may be amide (—CONH₂) or ester (—COOR). When the partial peptide of the present invention has a carboxyl group (or, carboxylate) at positions other than the C-terminus, those with an amidated or esterified carboxyl group are also included in the partial peptide of the present invention. An ester that is used in this case is, for example, the above ester at the C-terminus. Further, the partial peptide of the present invention also includes, for example, a peptide wherein an amino group of the amino acid residue at the N-terminus (for example, a methionine residue) is protected by a protecting group, a peptide wherein the glutamine residue, which is generated by in vivo cleavage of the N-terminal side, is pyroglutaminated, a peptide wherein a substituent on the side chain of amino acids within a molecule is protected by an appropriate protecting group, or a complex peptide, such as a so-called glycopeptide or the like to which sugar chains are bound. The partial peptide of the present invention can be used as an antigen to produce antibodies, so that it does not necessarily have activities of the polypeptide of the present invention.

The polynucleotide encoding the partial peptide of the present invention may be any polynucleotide, as long as it contains a nucleotide sequence encoding the above-described partial peptide of the present invention. The polynucleotide may be any of genomic DNA, cDNA derived from the above cells or tissues, and synthetic DNA. As the polynucleotide encoding the partial peptide of the present invention, for example, a polynucleotide having a partial sequence of any one of the nucleotide sequences of SEQ ID NOS: 1 to 11, or a polynucleotide hybridizing under stringent conditions to a polynucleotide having any one of the nucleotide sequences of SEQ ID NOS: 1 to 11 and having a partial nucleotide sequence of a polynucleotide encoding a polypeptide having characteristics substantially the same as the polypeptide of the present invention is used. The meaning of “a polynucleotide capable of hybridizing under stringent conditions to a polynucleotide having a partial sequence of any one of the nucleotide sequences of SEQ ID NOS: 1 to 11” is understood according to the above definition, “a polynucleotide capable of hybridizing under stringent conditions to a polynucleotide having any one of the nucleotide sequences of SEQ ID NOS: 1 to 11.” Hybridization methods and stringent conditions employed herein are similar to the methods and conditions described above.

The polynucleotide encoding the partial peptide of the present invention can be isolated by amplification by the PCR method using synthetic primers having partial nucleotide sequences of the polynucleotide encoding the partial peptide of the present invention, or can be isolated by screening a DNA library incorporated into an appropriate vector by hybridization using as a probe a labeled or unlabeled polynucleotide encoding a part of or the whole partial peptide of the present invention. Hybridization can be performed according to, for example, a method described in Molecular Cloning 2nd Edition (J. Sambrook et al., Cold Spring Harbor Lab. Press, 1989). Further, when a commercially available library is used, it can be performed according to a method described in the instructions attached thereto. The thus isolated polynucleotide can be used intact according to the purpose, or if desired, it can be used after digestion with restriction enzymes, or addition of linkers. The polynucleotide may have ATG as a translation initiation codon on the 5′ terminal side, and TAA, TGA or TAG as a translation termination codon on the 3′ terminal side. These translation initiation and translation termination codons can be added using appropriate synthetic DNA adaptors. Further, at 5′ or 3′ proximal to the translation initiation codon, or at 5′ proximal to the translation termination codon, an appropriate polynucleotide can be added to confirm the production of a partial peptide encoded by the polynucleotide, to purify the partial peptide, or to prepare fused partial peptides.

A recombinant vector or a recombinant expression vector which contains the polynucleotide encoding the partial peptide of the present invention can be produced by, for example, (a) excising a target DNA fragment from the polynucleotide encoding the partial peptide of the present invention, and (b) ligating the DNA fragment downstream of a promoter in an appropriate recombinant vector or a recombinant expression vector.

As salts of the polypeptide and the partial peptide of the present invention, salts of the same with physiologically acceptable acids (for example, inorganic acid, and organic acid), bases (for example, alkali metal salts), and the like are used. In particular, physiologically acceptable acid-added salts are preferred. Examples of such salts that are used herein include, salts of the polypeptide or the partial peptide with inorganic acid (for example, hydrochloric acid, phosphoric acid, hydrobromic acid, and sulfuric acid), or with organic acid (for example, acetic acid, formic acid, propionic acid, fumaric acid, maleic acid, succinic acid, tartaric acid, citric acid, malic acid, oxalate, benzoic acid, methanesulfonic acid, and benzenesulfonic acid). The polypeptide, partial peptide or salts thereof of the present invention can also be produced from the above cells or tissues of humans or warm-blooded animals by a method known per se for purifying a polypeptide and a partial peptide, and can also be produced according to a later-described method for synthesizing a peptide. Further, the polypeptide, partial peptide or salts thereof of the present invention can also be produced by culturing a transformant which contains the polynucleotide encoding a polypeptide or partial peptide, as described later. Further, the polypeptide, partial peptide or salts thereof of the present invention can also be produced by a cellular protein synthesis system. When the polypeptide, partial peptide or salts thereof of the present invention is produced from the tissues or cells of humans or mammals, the tissue or cells of humans or mammals are homogenized, the homogenized product is applied to extraction with acid or the like, and then the extract is subjected to a combination of chromatography, such as reverse phase chromatography, and ion exchange chromatography, so that purification and isolation can be performed.

To synthesize the polypeptide, partial peptide or amide thereof, or salts thereof, of the present invention, a commercial resin for protein (polypeptide) synthesis can be used normally. Examples of such a resin can include a chloromethyl resin, hydroxymethyl resin, benzhydrylamine resin, aminomethyl resin, 4-benzyloxy benzyl alcohol resin, 4-methylbenzhydrylamine resin, PAM resin, 4-hydroxymethyl methylphenyl acetamide methyl resin, polyacrylamide resin, 4-(2′, 4′-dimethoxyphenyl-hydroxymethyl) phenoxy resin, and 4-(2′, 4′-dimethoxyphenyl-Fmoc aminoethyl) phenoxy resin. Using such a resin, α-amino groups and amino acids wherein the side-chain functional groups are appropriately protected are condensed on the resin in accordance with the sequence of a target polypeptide according to various condensation methods known per se. Various protecting groups are removed at the same time as the excision of polypeptides from the resin at the end of the reaction. Then, reaction for intramolecular disulfide bond formation is performed in high dilution solution, so that the target polypeptide or partial peptide or the amidated polypeptide or peptide is obtained. Regarding the above condensation of protected amino acids, various reagents for activation that can be used for protein synthesis can be used. In particular, carbodiimides are preferred. As carbodiimides, DCC, N, N′-diisopropyl carbodiimide, N-ethyl-N′-(3-dimethylamino proryl) carbodiimide, and the like are used. In activation with these carbodiimides, a protected amino acid is directly added to a resin together with a racemization suppressing additive (for example, HOBt or HOOBt), or a protected amino acid is activated beforehand as symmetry acid anhydride or HOBt ester or HOOBt ester, and then can be added to the resin.

A solvent to be used for activation of protected amino acids or condensation with resin can be appropriately selected from solvents known to be applicable to protein condensation reaction. For example, acid amides, such as N,N-dimethylformamide, N,N-dimethylacetamide, and N-methylpyrrolidone; halogenated hydrocarbons, such as methylene chloride and chloroform; alcohols, such as trifluoroethanol; sulfoxides, such as dimethylsulfoxide; ethers, such as pyridine, dioxane, and tetrahydrofuran; nitriles, such as acetonitrile and propionitrile; esters, such as methyl acetate and ethyl acetate; or appropriate mixtures thereof, are used. Reaction temperature is appropriately selected from a range known to be applicable to reaction of protein bond formation. The reaction temperature is normally appropriately selected from a range of approximately −20° C. to 50° C. Activated amino acid derivatives are normally used in 1.5 to 4-fold excess. When condensation is insufficient as tested using ninhydrin reaction, condensation reaction is repeated without eliminating protecting groups, so that condensation can be performed sufficiently. When sufficient condensation cannot still be obtained even when the reaction is repeated, unreacted amino acids are acetylated using acetic anhydride or acetylimidazole, and thus it is possible to prevent a successive reaction from being affected.

As protecting groups of amino groups of raw materials, for example, Z, Boc, t-pentyloxy carbonyl, isobornyloxycarbonyl, 4-methoxy benzyloxycarbonyl, C1-Z, Br-Z, adamantyloxycarbonyl, trifluoroacetyl, phthaloyl, formyl, 2-nitrophenylsulphenyl, diphenyl phosphinothioyl, Fmoc, and the like are used. Carboxyl groups can be protected by, for example, alkyl esterification (for example, linear, branched or cyclic alkyl esterification of methyl, ethyl, propyl, butyl, t-butyl, cyclopentyl, cyclohexyl, cycloheptyl, cyclooctyl, or 2-adamanthyl), aralkyl esterification (for example, benzyl esterification, 4-nitrobenzyl esterification, 4-methoxybenzyl esterification, 4-chlorobenzyl esterification, and benzhydryl esterification), phenacyl esterification, benzyloxycarbonyl hydrazide formation, t-butoxycarbonyl hydrazide formation, trityl hydrazide formation and the like. Hydroxyl groups of serine can be protected by, for example, esterification or etherification. As groups appropriate for esterification, for example, a lower (C1-6) alkanoyl group, such as acetyl groups, an aroyl group, such as benzoyl groups, and groups induced from carbons, such as benzyloxycarbonyl groups, and ethoxycarbonyl groups are used. Further, examples of a group appropriate for etherification include a benzyl group, tetrahydropyranyl group, t-butyl group and the like. As a protecting group of a phenolic hydroxyl group of tyrosine, for example, Bzl, Cl₂-Bzl, 2-nitrobenzyl, Br-Z, and t-butyl are used. As a protecting group of imidazole of histidine, for example, Tos, 4-methoxy-2,3,6-trimethylbenzenesulfonyl, DNP, benzyloxymethyl, Bum, Boc, Trt, and Fmoc are used.

As activated carboxyl groups of raw materials, for example, corresponding acid anhydrides, azides, and active esters (ester of the carboxyl group with alcohol, such as pentachlorophenol, 2,4,5-trichlorophenol, 2,4-dinitrophenol, cyanomethyl alcohol, para-nitrophenol, HONB, N-hydroxysuccinimide, N-hydroxyphthalimide, and HOBt) are used. As activated amino groups of raw materials, for example, corresponding phosphoric acid amides are used. As a removal (elimination) method of protecting groups, for example, contact reduction in the airstream of hydrogen in the presence of a catalyst such as Pd-black or Pd-carbon, acid treatment using anhydrous hydrofluoric acid, methanesulfonic acid, trifluoromethane sulfonic acid, trifluoroacetic acid, a mixed solution thereof or the like, base treatment using diisopropyl ethylamine, triethylamine, piperidine, piperazine or the like, or reduction using sodium in liquid ammonia is used. Generally, elimination reaction by the above acid treatment is performed at a temperature from approximately −20° C. to 40° C. In the acid treatment, for example, addition of a cation scavenger such as anisole, phenol, thioanisole, metacresol, paracresol, dimethylsulfide, 1,4-butane dithiol, or 1,2-ethanedithiol is effective. In addition, a 2,4-dinitrophenyl group used as an imidazole protecting group of histidine is removed by thiophenol treatment, a formyl group used as an indole protecting group of tryptophan is removed by alkali treatment using a diluted sodium hydroxide solution, diluted ammonia, or the like, in addition to deprotection by acid treatment in the presence of the above 1,2-ethanedithiol, 1,4-butane dithiol or the like.

Means for protecting functional groups that should not be involved in reaction of raw materials and the protecting groups, means for eliminating the protecting groups, means for activating functional groups involved in the reaction, and the like can be appropriately selected from known groups or known means. Another method for obtaining amides of polypeptides, for example, involves protecting the α-carboxyl group of a carboxy-terminal amino acid by amidation, allowing a peptide chain to elongate on the amino group side to a desired chain length, producing a polypeptide from which only the protecting group of a-amino group on the N-terminus of the peptide chain is removed and a polypeptide from which only the protecting group of the carboxyl group on the C-terminus is removed, and then condensing both polypeptides in the above mixed solvent. Details about the condensation reaction are as described above. The protected polypeptides obtained by condensation are purified, and then all the protecting groups are removed by the above method, so that desired crude polypeptides can be obtained. The crude polypeptides are purified by freely using various known means of purification, and then the main fraction is freeze-dried, so that the amides of the desired polypeptides can be obtained. To obtain esters of polypeptides, for example, the α-carboxyl groups of carboxy-terminal amino acids are condensed with desired alcohols to form amino acid esters, and then esters of desired polypeptides can be obtained by a method similar to that employed to obtain amides of polypeptides.

The partial peptide or a salt thereof of the present invention can be produced according to a method known per se for peptide synthesis, or by cleaving the polypeptide of the present invention with appropriate peptidase. A method for synthesizing peptides may be, for example, either a solid phase synthesis method or a liquid phase synthesis method. Specifically, a target partial peptide can be produced by condensing partial peptides or amino acids that can form the partial peptide of the present invention with remaining regions, and then eliminating protecting groups when the product contains protecting groups. Examples of known condensation methods or methods for eliminating protecting groups include the following methods: M. Bodanszky and M. A. Ondetti: Peptide Synthesis, Interscience Publishers, New York (1966), Schroeder and Luebke: The Peptide, Academic Press, New York (1965), and Haruaki YAJIMA and Shunpei SAKAKIBARA: Biochemistry Experimental lecture 1, Protein Chemistry IV (1977). Further, after reaction, the partial peptide of the present invention can be purified and isolated by a normal purification method, for example, a combination of solvent extraction, distillation, column chromatography, liquid chromatography, recrystallization and the like. When the partial peptides obtained by the above method are free peptides, they can be converted into appropriate salts by any known method or any method according to the known method. Conversely, when the partial peptides are obtained as salts, they can be converted into free peptides or other salts by any known method or any method according to the known method.

When the polypeptide or the partial peptide of the present invention is produced by culturing transformants containing polynucleotides encoding the polypeptides or the partial peptides of the present invention, and when the hosts are bacteria of the genus Escherichia or the genus Bacillus, an appropriate medium to be used for culturing the transformant is a liquid medium. The medium may contain a carbon source, nitrogen source, minerals, and the like required for the growth of the transformant. Examples of a carbon source include glucose, dextrin, soluble starch, sucrose, and the like; examples of a nitrogen source include inorganic or organic substances, such as ammonium salts, nitrates, corn steep liquor, peptone, casein, meat extract, soybean cake, and potato extract; and examples of minerals include calcium chloride, sodium dihydrogenphosphate, and magnesium chloride. In addition, yeast extract, vitamins, growth-promoting factors, and the like may be added. The pH of a medium preferably ranges from approximately 5 to 8.

As a medium for culturing bacteria of the genus Escherichia, for example, a M9 medium containing glucose and casamino acid is preferable. If necessary, for example, an agent such as 3β-indolyl acrylic acid can be added to allow promoters to function efficiently. When bacteria of the genus Escherichia are used as a host, culturing is normally performed at approximately 15 to 43° C. for about 3 to 24 hours. If necessary, aeration and agitation may also be performed. When bacteria of the genus Bacillus are used as a host, culturing is normally performed at approximately 30 to 40° C. for about 6 to 24 hours. If necessary, aeration and agitation may also be performed. When transformants whose host is yeast are cultured, examples of a medium include Burkholder minimal media (Proc. Natl. Acad. Sci. USA, 77; 4505, 1980) and SD media (Proc. Natl. Acad. Sci. USA, 81; 5330, 1984) containing 0.5% casamino acid. The pH of a medium is preferably adjusted to be approximately 5 to 8. Culturing is normally performed at approximately 20 to 35° C. for about 24 to 72 hours. If necessary, aeration and agitation may also be performed. When transformants whose host is an insect cell or insect are cultured, a medium used herein is, for example, Grace's Insect Medium (Nature, 195; 788, 1962) appropriately supplemented with immobilized additives, such as 10% fetal calf serum. The pH of a medium is preferably adjusted to be approximately 6.2 to 6.4. Culturing is normally performed at approximately 27° C. for about 3 to 5 days. If necessary, aeration and agitation may also be performed. When transformants whose host is an animal cell are cultured, examples of a medium used herein include MEM media containing approximately 5 to 20% fetal calf serum (Science, 122; 501, 1952), DMEM media (Virology, 8; 396, 1959), RPMI 1640 media (J. American Med. Assoc., 199; 519, 1967), and 199 media (Proc. Soci. Biol. Med., 73; 1, 1950). The pH preferably ranges from approximately 6 to 8. Culturing is normally performed at approximately 30 to 40° C. for about 15 to 60 hours. If necessary, aeration and agitation are performed. As described above, the polypeptide or the partial peptide of the present invention can be generated within the cells, on the cell membranes, or outside the cells of the transformants.

The polypeptide or the partial peptide of the present invention can be separated and purified from the above culture product by, for example, the following methods. When the polypeptide or the partial peptide of the present invention is extracted from the cultured microbes or the cells, a method appropriately used herein involves collecting microbes or cells by any known method after culturing, suspending them in an appropriate buffer, disrupting the microbes or the cells by ultrasonication, lysozyme treatment, and/or freezing and thawing, and obtaining a crude extract of the polypeptide or the partial peptide by centrifugation or filtration. A protein denaturation agent, such as urea or guanidine hydrochloride, or a surfactant, such as Triton X-100™, may be contained in the buffer. When the polypeptide or the partial peptide is secreted in a culture solution, the microbes or the cells are separated from the supernatant by a method known per se after culturing, so as to collect the supernatant. The polypeptide or the partial peptide contained in the thus obtained culture supernatant or the extract can be purified by an appropriate combination of separation and purification methods known per se. Examples of these known separation and purification methods used herein include a method utilizing solubility, such as a salting out method or a solvent precipitation method, a method mainly utilizing differences in molecular weight, such as dialysis, ultrafiltration, gel filtration or SDS-polyacrylamide gel electrophoresis, a method utilizing differences in electric charge, such as ion exchange chromatography, a method utilizing specific affinity, such as affinity chromatography, a method utilizing differences in hydrophobicity, such as reverse phase high performance liquid chromatography, and a method utilizing differences in isoelectric point, such as an isoelectric focusing method.

When the thus obtained polypeptides or the partial peptides are free polypeptides or partial peptides, they can be converted into salts by a method known per se or a method according thereto. Conversely, when the polypeptides or the partial peptides are obtained as salts, they can be converted into free polypeptides or peptides, or other salts by a method known per se or a method according thereto. The polypeptides or the partial peptides produced by recombinants can be freely modified by allowing, before or after purification, an appropriate protein modification enzyme to act thereon or the polypeptides can also be partially removed. As a protein modification enzyme, for example, trypsin, chymotrypsin, arginyl endopeptidase, protein kinase, and glycosidase are used. The presence of the thus generated polypeptide or partial peptide or salts thereof of the present invention can be measured by enzyme immunoassay, Western Blotting, or the like using specific antibodies.

Obtainment of Antibody Against the Polypeptide, Partial Peptide or Salts Thereof of the Present Invention

The antibody against the polypeptide, partial peptide, or salts thereof of the present invention may be either a polyclonal antibody or monoclonal antibody, as long as it can recognize the polypeptide, partial peptide, or salts thereof of the present invention. An antibody against the polypeptide, partial peptide, or salts thereof of the present invention (hereinafter, in descriptions for the antibody, they are abbreviated as simply “the peptide of the present invention”) can be produced according to a method known per se for producing antibodies or anti-sera using the peptide of the present invention as an antigen.

Preparation of Monoclonal Antibody

(a) Preparation of Monoclonal Antibody-Producing Cell

The peptide of the present invention is administered alone or together with a carrier and diluent to the site of a warm-blooded animal which is capable of producing antibodies in response to administration. To enhance antibody-producing ability, an adjuvant such as a complete Freund's adjuvant or incomplete Freund's adjuvant may be administered upon administration. Administration is performed normally once every 2 to 6 weeks for about 2 to 10 times in total. Examples of a warm-blooded animal used herein include a monkey, rabbit, dog, guinea pig, mouse, rat, sheep, goat, and chicken. A mouse and rat are preferably used. Upon preparation of monoclonal antibody-producing cells, individuals recognized to have antibody titer are selected from warm-blooded animals such as mice, immunized with antigen, spleens or lymph glands are collected on day 2 to 5 after the final immunization, antibody-producing cells contained therein are allowed to fuse with myeloma cells of animals of the same species or of a different species, so that monoclonal antibody-producing hybridomas can be prepared. Antibody titer in anti-sera can be measured by allowing labeled peptides described later to react with anti-sera, and then measuring activity of a labeling agent bound to antibodies. A fusion procedure can be performed by any known method, for example, by Kohler and Millstein's method (Nature, 256; 495, 1975). Examples of a fusion promoting agent include polyethylene glycol (PEG) and Sendai virus. Preferably, PEG, and more preferably PEG 1000 to PEG 6000 are used.

Examples of myeloma cells include myeloma cells of warm-blooded animals, such as NS-1, P3U1, SP2/0, and AP-1. Preferably, P3U1 is used. A preferable ratio employed herein of the number of antibody-producing cells (spleen cells) to the number of myeloma cells ranges from about 1:1 to 20:1. PEG is added at a concentration of approximately 10 to 80%, and incubation is performed at 20 to 40° C., and preferably at 30 to 37° C. for 1 to 10 minutes, so that cell fusion can be performed efficiently. Various methods can be used for screening for monoclonal antibody-producing hybridomas. Examples of such a method include a method which involves adding a hybridoma culture supernatant to a solid phase (for example, a microplate) to which peptide antigens are adsorbed directly or adsorbed together with carriers, and then adding anti-immunoglobulin antibodies labeled with radioactive substances or enzymes (when mouse cells are used for cell fusion, anti-mouse immunoglobulin antibodies are used) or adding protein A, thereby detecting monoclonal antibodies bound to the solid phase; and a method which involves adding a hybridoma culture supernatant to a solid phase to which anti-immunoglobulin antibodies or protein A is adsorbed, and then adding protein labeled with radioactive substances or enzymes, thereby detecting monoclonal antibodies bound to the solid phase. Monoclonal antibodies can be selected according to a method known per se or a method according thereto. Normally, selection can be performed in a medium for animal cells supplemented with HAT (hypoxanthine, aminopterin, and thymidine). As media for selection and breeding, any medium can be used as long as it enables the growth of hybridomas. For example, 1 to 20%, and preferably 10 to 20% fetal calf serum-containing RPMI 1640 medium, serum-free medium for culturing hybridomas (SFM-101, NISSUI PHARMACEUTICAL), or the like can be used. The temperature for culturing normally ranges from 20 to 40° C., and is preferably approximately 37° C. The time for culturing normally ranges from 5 days to 3 weeks, and preferably 1 to 2 weeks. Culturing can be normally performed in the presence of 5% carbon dioxide gas. Antibody titer of a hybridoma culture supernatant can be measured in a manner similar to the above measurement of antibody titer in anti-sera.

(b) Purification of Monoclonal Antibody

Monoclonal antibodies can be separated and purified according to a method known per se, such as a method for separating and purifying immunoglobulin [for example, salting-out, alcohol precipitation, isoelectric precipitation, electrophoresis, adsorption and desorption method using ion exchanger (for example, DEAE), ultracentrifugation, gel filtration, and specific purification which involves collecting only antibodies using an antigen-bound solid phase or active adsorbent, such as protein A or protein G, and then dissociating bonds, so as to obtain antibodies.

Preparation of Polyclonal Antibody

The polyclonal antibody of the present invention can be produced by a method known per se or a method according thereto. For example, the polyclonal antibody can be produced by preparing an immunogen itself, or a complex of the immunogen and a carrier protein, immunizing a warm-blooded animal in a manner similar to the above method for producing monoclonal antibodies, collecting products containing antibodies against the peptide of the present invention from the immunized animal, and then separating and purifying antibodies. Regarding a complex of an immunogen and a carrier protein to be used for immunizing a warm-blooded animal, any type of the carrier protein and any mixing ratio of the carrier to a hapten can be used for cross-linking, as long as antibodies can be efficiently produced in response to haptens cross-linked to the carriers for immunization. For example, a method which involves allowing bovine serum albumin, bovine thyroglobulin, hemocyanin or the like to conjugate to a hapten at a ratio by weight of approximately 0.1 to 20 to hapten 1, and preferably approximately 1 to 5 to hapten 1 is used. In addition, in conjugation of a hapten and a carrier, various condensing agents can be used. An active ester agent or the like containing glutaraldehyde, carbodiimide, maleimide active ester, thiol group, or dithioviridyl group is used. A condensation product is administered by itself or together with a carrier, diluent or the like to a site of a warm-blooded animal at which antibody production is possible. To enhance antibody-producing ability, upon administration, a complete Freund's adjuvant or an incomplete Freund's adjuvant may be administered. Administration is normally performed once every about 2 to 6 weeks for about 3 to 10 times in total. Polyclonal antibodies can be collected from blood, ascites fluid, or the like of a warm-blooded animal immunized by the above method, and collection from blood is preferable. The antibody titer of polyclonal antibodies in an anti-serum can be measured in a manner similar to the above measurement of the antibody titer in the anti-serum. Polyclonal antibodies can be separated and purified according to a method for separating and purifying immunoglobulin similar to the above method for separating and purifying monoclonal antibodies.

Production of Transgenic Animal

The transgenic animal of the present invention can be produced by techniques known by a person skilled in the art to produce transgenic animals. For example, when the polynucleotide of the present invention or a polynucleotide encoding the polypeptide of the present invention is a target gene, the target gene is introduced into the genome of a target animal, so as to cause overexpression of the target gene within the genome, or when an endogenous target gene sequence is present, overexpression of the target gene, or disruption of the endogenous target gene makes it possible to cause underexpression of the endogenous target gene or to inactivate the expression thereof.

The present invention can provide non-human mammals having the foreign polynucleotide of the present invention (hereinafter, referred to as the foreign polynucleotide of the present invention) or the mutant polynucleotide thereof (may also be referred to as the foreign mutant polynucleotide of the present invention”). Specifically, the present invention provides (1) a non-human mammal having the foreign polynucleotide of the present invention or the mutant polynucleotide thereof, (2) the animal of (1) above, wherein the non-human mammal is a rodent, (3) the animal of (2) above, wherein the rodent is a mouse or rat, and (4) a recombinant vector which contains the foreign polynucleotide or the mutant polynucleotide thereof of the present invention, and can be expressed in a mammal. The non-human mammal (hereinafter, referred to as “the transgenic animal of the present invention”) having the foreign polynucleotide or the mutant polynucleotide thereof of the present invention can be produced by transferring a target DNA into germinal cells or the like including unfertilized eggs, fertilized eggs, sperms and the progenitor cells thereof, preferably, at the stage of embryogenesis in the development of a non-human mammal (more preferably, at the stage of single cell or fertilized egg cell, and generally before 8-cell phase) by a calcium phosphate method, electric pulse method, lipofection method, aggregation method, microinjection method, particle gun method, DEAE-dextran method or the like. Further, the target foreign polynucleotide of the present invention is transferred to somatic cells, organs in vivo, tissue cells or the like by this transfer method of DNA, so that it can be used for cell culture, tissue culture and the like. Furthermore, these cells are fused with the above germinal cells by a cell fusion method known per se, so as to be able to produce the transgenic animal of the present invention.

As nonhuman mammals, for example, cattle, pigs, sheep, goats, rabbits, dogs, cats, guinea pigs, hamsters, mice, and rats are used. Above all, rodents that have relatively short ontogenic and biological cycles in terms of producing a disease animal model system and can be easily bred, particularly mice (for example, pure-bred lines, such as a C57BL/6 line and DBA2 line; and cross-bred lines, such as B6C3F1 line, BDF1 line, B6D2FI line, BALB/c line, and ICR line) or rats (for example, Wistar line and SD line) are preferred. An example of “a mammal” in which a recombinant vector can be expressed is a human, in addition to the above non-human mammals. The foreign polynucleotide of the present invention is not the polynucleotide of the present invention that a non-human mammal originally possesses, but is the polynucleotide of the present invention which is isolated and extracted once from the mammal. As the mutant polynucleotide of the present invention, one wherein an alteration (for example, mutation) has occurred in the original nucleotide sequence of the polynucleotide of the present invention is used. Specifically, the polynucleotide or the like wherein addition or deletion of nucleotides or substitution with other nucleotides has occurred is used. In addition, an abnormal polynucleotide is also included in the mutant polynucleotide of the present invention. The abnormal polynucleotide refers to a polynucleotide which allows the abnormal polypeptide of the present invention to be expressed. For example, a polynucleotide and the like allowing the expression of a polypeptide which suppresses the function of the normal polypeptide of the present invention is also used. In this specification, “normal” used for the polypeptide of the present invention means that the polypeptide can express its original functions. In this specification, “abnormal” used for the polypeptide of the present invention means that the polypeptide does not express functions qualitatively or quantitatively equivalent to the original functions. The foreign polynucleotide of the present invention may be derived from a mammal of either the same species or a different species from a target animal. When the polynucleotide of the present invention is transferred to a target animal, it is generally advantageous that the polynucleotide is used as a DNA construct which is ligated downstream of a promoter which enables the expression of the polynucleotide in animal cells. For example, when the human polynucleotide of the present invention is transferred, a DNA construct (for example, a vector) which has the human polynucleotide of the present invention ligated downstream of each type of promoter that enables the expression of a polynucleotide derived from each type of mammal (for example, rabbits, dogs, cats, guinea pigs, hamsters, rats, and mice) having the polynucleotide of the present invention that has high homology with the nucleotide sequence is micro-injected into a fertilized egg of a target mammal, such as a mouse fertilized egg, so that a transgenic mammal that highly expresses the human polynucleotide of the present invention can be produced.

As an expression vector for the polypeptide of the present invention when the transgenic mammal of the present invention is produced, a plasmid derived from Escherichia coli, a plasmid derived from Bacillus subtilis, a plasmid derived from yeast, a bacteriophage, such as X phage, a retrovirus, such as Moloney leukemia virus, and an animal virus, such as vaccinia virus, or Baculovirus are used. Among these, a plasmid derived from Escherichia coli, a plasmid derived from Bacillus subtilis, or a plasmid derived from yeast is preferably used. As a promoter to regulate the expression of the above DNA, for example, promoters of DNA derived from a virus (for example, a simian virus, cytomegalovirus, Moloney leukemia virus, JC virus, mammary tumor virus, and poliomyelitis virus), and promoters derived from various mammals (humans, rabbits, dogs, cats, guinea pigs, hamsters, rats, mice, and the like), for example, albumin, insulin II, uroplakin II, elastase, erythropoietin, endothelin, muscle creatine kinase, glial fibrillary acidic protein, glutathione S-transferase, platelet-derived growth factor β, keratin K1, K10 and K14, collagen type I and II, cyclic AMP-dependent protein kinase βI subunit, dystrophin, tartarate-resistant alkaline phosphatase, atrial natriuretic factor, endothelial receptor tyrosine kinase (generally abbreviated as Tie2), sodium-potassium adenosine triphosphatase (Na, K-ATPase), neurofilament light chain, metallothionein I and IIA, metalloproteinase 1 tissue inhibitor, MHC class I antigen (H-2 L), H-ras, renin, dopamine β-hydroxylase, thyroidal peroxidase (TPO), polypeptide chain elongation factor 1α (EF-1α), β actin, α and β myosin heavy chain, myosin light chain 1 and 2, myelin basic protein, thyroglobulin, Thy-1, immunoglobulin, H chain variable portion (VNP), blood serum amyloid P component, myoglobin, troponin C, smooth muscle α actin, preproenkephalin A, and vasopressin are used. Among these, promoters that can be highly expressed in a whole body, such as cytomegalovirus promoters, human polypeptide chain elongation factor 1α (EF-1α) promoters, and human and chicken β actin promoters, are preferred.

The above vector preferably has a sequence that terminates the transcription of a target messenger RNA (generally referred to as a terminator) in a transgenic mammal. For example, each sequence of DNAs derived from viruses and various mammals can be used, and preferably, for example, SV40 terminator of simian virus is used. In addition, for the purpose of causing the higher expression of a target foreign polynucleotide, splicing signals of each polynucleotide, an enhancer region, a part of introns of eukaryotic DNA, and the like can be ligated 5′ upstream of a promoter region, between a promoter region and a translation region, or 3′ downstream of a translation region, depending on the purpose. The translation region can be prepared as a DNA construct that can be expressed in a transgenic animal by a normal genetic engineering technique which ligates the region downstream of the above promoter and if necessary, upstream of a transcription termination site. The transfer of the foreign polynucleotide of the present invention in the stage of fertilized egg cells is ensured so that the polynucleotides are present in all the germinal and somatic cells of a target mammal. In germinal cells of the thus produced animal after the transfer of the polynucleotide, the presence of the foreign polynucleotide of the present invention means that all the progenies of the produced animal retain the foreign polynucleotide of the present invention in all the germinal and somatic cells thereof. Progenies of this species of animals that have inherited the foreign polynucleotide of the present invention have the foreign polynucleotide of the present invention in all the germinal and somatic cells.

A non-human mammal to which the foreign normal polynucleotide of the present invention has been transferred can be successively bred under normal breeding environment as an animal having the polynucleotide, after the stable retention of the foreign polynucleotide is confirmed by crossing. The transfer of the foreign polynucleotide of the present invention in the stage of fertilized egg cells is ensured so that the foreign polynucleotide is excessively present in all the germinal and somatic cells of a target mammal. In germinal cells of the thus produced animal after the transfer of the polynucleotide, the excessive presence of the foreign polynucleotide of the present invention means that all the progenies of the produced animals excessively have the foreign polynucleotide of the present invention in all the germinal and somatic cells thereof. Progenies of this species of animals that have inherited the foreign polynucleotide of the present invention excessively have the foreign polynucleotide of the present invention in all the germinal and somatic cells. Homozygote animals having the introduced polynucleotide in both homologous chromosomes are obtained, and then the female and male of these animals are crossed, so that the animals can be successively bred such that all the progenies excessively have the polynucleotide. In non-human mammals having the normal polynucleotide of the present invention, the normal polynucleotide of the present invention is highly expressed. Thus, when the function of the endogenous normal polynucleotide is promoted the mammals may finally develop hyperergasia of the polypeptide of the present invention, so that the mammals can be used as pathological model animals thereof. In this specification, the hyperergasia of a polypeptide means a state in which physiological functions of the polypeptide are enhanced mainly due to an increase in the content of the polypeptide in vivo, in tissues or within cells, so that a pathologic state is developed or a disease is induced. For example, tumor formation caused by the overexpression of oncogene, hypersensitivity against inflammatory pain caused by overexpression of NMDA receptor NR2B subunit, and the like have already been reported concerning mice. For example, using the normal polynucleotide-transferred animals of the present invention, pathological mechanisms of hyperergasia of the polypeptide of the present invention and diseases relating to the polypeptide of the present invention can be elucidated and the therapies against these diseases can be studied. Further, since mammals having the foreign normal polypeptide of the present invention transferred thereto exhibit a symptom of an increase in the polypeptide of the present invention in a free form, they can be utilized for tests for screening for a therapeutic agent against diseases relating to an increase in the polypeptide of the present invention. Furthermore, the normal polynucleotide-transferred animals of the present invention can be utilized to produce the polypeptide of the present invention.

On the other hand, a non-human mammal having the foreign abnormal polynucleotide of the present invention can be successively bred under normal breeding environment as an animal having the polynucleotide, after the stable retention of the foreign abnormal polynucleotide is confirmed by crossing. Further, a target foreign abnormal polynucleotide that is incorporated into the above plasmid can be used as a source of a foreign abnormal polynucleotide to be genetically engineered. A DNA construct of a foreign abnormal polynucleotide with a promoter can be prepared by normal gene engineering techniques. The transfer of the foreign abnormal polynucleotide of the present invention in the stage of fertilized egg cells is ensured so that the polynucleotide is present in all the germinal and somatic cells of a target mammal. In germinal cells of the thus produced animal after the transfer of the abnormal polynucleotide, the presence of the abnormal polynucleotide of the present invention means that all the progenies of the produced animals have the foreign abnormal polynucleotide of the present invention in all the germinal and somatic cells thereof. Progenies of such the animals that have inherited the foreign abnormal polynucleotide of the present invention have the foreign abnormal polynucleotide of the present invention in all the germinal and somatic cells. Homozygote animals having the introduced foreign abnormal polynucleotide in both homologous chromosomes are obtained, and then the female and male animals are crossed, so that the animals can be successively bred such that all the progenies have the abnormal polynucleotide. In non-human mammals having the abnormal polynucleotide of the present invention, the abnormal polynucleotide of the present invention is highly expressed. Thus, when the function of the endogenous normal polynucleotide is inhibited the mammals may finally develop function-inactivated type unresponsiveness of the polypeptide of the present invention, so that the mammals can be used as pathological model animals thereof. In this specification, “function-inactivated type unresponsiveness of a polypeptide” means a state in which physiological functions of the polypeptide are decreased or become unable to function mainly due to a decrease in the content of the normal polypeptide of the present invention in vivo, in tissues or within cells, or due to defect in the normal polypeptide of the present invention, so that a pathologic state is developed or a disease is induced. An example of such a state in mice is hyperplasia of mammary gland epithelium caused by the overexpression of transforming growth factor receptor deficient in kinase activity (Cell Growth Differ., 9, 229, 1998). For example, using the abnormal polynucleotide-transferred animals of the present invention, the pathological mechanism of function-inactivated type unresponsiveness of the polypeptide of the present invention can be elucidated, and diseases relating to the polypeptide of the present invention and the therapies against these diseases can be studied. As a specific possible application, the animal highly expressing the abnormal polynucleotide of the present invention can be a model to elucidate the inhibited function (Dominant Negative action) of the normal polypeptide by the abnormal polypeptide of the present invention in function-inactivated type unresponsiveness of the polypeptide of the present invention. Further, since mammals having the foreign abnormal polynucleotide of the present invention transferred thereto have a symptom of an increase in the polypeptide of the present invention in a free form, they can be utilized for tests for screening for a therapeutic agent against function-inactivated type unresponsiveness of the polypeptide of the present invention.

Examples of possible applications for other purposes of the above 2 types of transgenic animals of the present invention include (1) use as a cell source for tissue culture, (2) analysis on the relationship with protein which is specifically expressed or activated by the polypeptide of the present invention by direct analysis of DNA or RNA in the tissue of the transgenic animal of the present invention or analysis of the protein structure expressed by DNA, (3) study on the function of the cells from tissues, which are generally difficult to culture, using the cells of tissue having DNA cultured by a standard tissue culture technique, (4) screening for a drug or the like that enhances the function of cells by the use of the cells described in (1) above, and (5) isolation and purification of the mutant polypeptide of the present invention and the preparation of the antibody thereof. Moreover, by the use of the transgenic animal of the present invention, clinical symptoms of diseases relating to the polypeptide of the present invention including function-inactivated type unresponsiveness of the polypeptide of the present invention can be examined, and detailed pathological findings in each organ of models of diseases relating to the polypeptide of the present invention can be obtained. Hence, the use of the transgenic animal can contribute to the development of a new therapeutic method, and further contribute to study and treatment for secondary diseases due to the diseases. Further, each organ is excised from the transgenic animal of the present invention and then shredded, so that the transgenic cells separated by protease, such as trypsin, can be obtained and cultured, or the cultured cells can be organized. Moreover, the cells producing the polypeptide of the present invention are, for example, specified, so that the cells can be effective research materials for elucidating the polypeptide of the present invention and its action. Further, the use of the transgenic animal of the present invention makes it possible to provide an effective and rapid method for screening for the therapeutic agent against the disease relating to the polypeptide of the present invention including function-inactivated type unresponsiveness, in order to develop the therapeutic agent. Furthermore, the use of the transgenic animal of the present invention or the foreign polynucleotide expression vector of the present invention makes it possible to study and develop a therapeutic method using the polynucleotide of the disease relating to the polypeptide of the present invention.

The present invention provides embryonic stem cells of a non-human mammal wherein the polynucleotide of the present invention is inactivated, and non-human mammals which are deficient in expression of the polynucleotide of the present invention. That is, the present invention provides (1) embryonic stem cells of a non-human mammal wherein the polynucleotide of the present invention is inactivated, (2) the embryonic stem cells described in (1) wherein the polynucleotide is inactivated by introducing a reporter gene (for example, P-galactosidase gene derived from Escherichia coli), (3) the embryonic stem cells described in (1), which are neomycin-resistant, (4) the embryonic stem cells described in (1), wherein the non-human mammal is a rodent, (5) the embryonic stem cells described in (4), wherein the rodent is a mouse, (6) a non-human mammal that is deficient in expression of the polynucleotide, wherein the polynucleotide of the present invention is inactivated, (7) the non-human mammal described in (6), wherein the polynucleotide is inactivated by introducing a reporter gene (for example, β-galactosidase gene derived from Escherichia coli), and the reporter gene can be expressed under regulation of a promoter for the polynucleotide of the present invention, (8) the non-human mammal described in (6), wherein the non-human mammal is a rodent, (9) the non-human mammal described in (8), wherein the rodent is a mouse, and (10) a method for screening for a compound or a salt thereof that promotes or inhibits the promoter activity for the polynucleotide of the present invention, which is characterized by administering a test compound to the animal described in (7), and detecting the expression of the reporter gene.

“Embryonic stem cells of a non-human mammal wherein the polynucleotide of the present invention is inactivated” refers to embryonic stem cells (hereinafter, abbreviated as “ES cells”) of a non-human mammal (hereinafter may also be referred to as “the knockout DNA of the present invention”) wherein the ability of expressing the polynucleotide is suppressed due to artificial addition of a mutation to the polynucleotide of the present invention that the non-human mammal has, or wherein the polynucleotide does not substantially have an ability of expressing the polypeptide of the present invention due to substantial loss of the activity of the polypeptide of the present invention encoded by the polynucleotide. Animals similar to those described above are used as non-human mammals. A method for artificially adding a mutation to the polynucleotide of the present invention can be performed by, for example, deleting a part of or the entire sequence of the polynucleotide, or inserting or substituting with another polynucleotide using gene engineering techniques. With these mutations, the knockout DNA of the present invention may be prepared by, for example, shifting the reading frame of codons or disrupting the function of a promoter or exon. Specific examples of the embryonic stem cells of a non-human mammal wherein the polynucleotide of the present invention is inactivated (hereinafter, abbreviated as “the polynucleotide-inactivated ES cells of the present invention” or “the knockout ES cells of the present invention”) include those obtained by, for example, preparing a polynucleotide chain (hereinafter, abbreviated as “a targeting vector”) having a polynucleotide sequence that is so constructed as to disrupt a gene as a result of: isolating the polynucleotide of the present invention that a target non-human mammal has, inserting, for example, a drug resistance gene represented by a neomycin resistance gene or hygromycin resistance gene, or a reporter gene represented by lacZ (β-galactosidase gene) or CAT (chloramphenicol acetyltransferase gene) into the exon region so as to disrupt the function of the exon, or inserting a polynucleotide sequence (for example, poly-A addition signal) that terminates the transcription of a gene into the intron region between the exons, so as to make it impossible to synthesize a complete messenger RNA; and then introducing the polynucleotide chain into the chromosome of the animal by, for example, a homologous recombination technique, analyzing the thus obtained ES cells by Southern hybridization analysis using as a probe the polynucleotide sequence on or in the vicinity of the polynucleotide of the present invention, or by the PCR method using as primers the polynucleotide sequence on the targeting vector and the polynucleotide sequence in the neighboring region other than the polynucleotide of the present invention used for preparing the targeting vector, and then selecting the knockout ES cells of the present invention.

In addition, for example, as original ES cells in which the polynucleotide of the present invention is inactivated by a homologous recombination method or the like, previously established cells as described above, or cells that are newly established by any known method may be used. For example, in the case of ES cells of mice, ES cells of line 129 are currently used generally. However, since the immunological background of these cells is not clear, instead of this cell line, for the purpose of obtaining ES cells of a pure line with a genetic background which is immunologically clear, for example, cells established using BDF1 mice (F1 from C57BL/6 and DBA/2) which are produced by improving, by crossing with DBA/2, and are more improved than C57BL/6 mice or C57BL/6 in terms of the small number of eggs collected, can be preferably used. In addition to the advantages that many eggs can be collected and the collected eggs are healthy, BDF1 mice have C57BL/6 mice in their background. Thus, when model mice are produced, ES cells obtained using BDF1 mice can be used advantageously in that their genetic background can be substituted with C57BL/6 mice by back-crossing with C57BL/6 mice. Further, when ES cells are established, blastocysts at 3.5 days after fertilization are generally used. In addition to these cells, 8-cell stage embryos are collected, cultured to be blastocysts, and then used, so that a large number of initial embryos can be obtained efficiently. Further, ES cells of both female and male animals may be used. In general, ES cells of a male animal are convenient for producing germ line chimera. In addition, it is desirable to distinguish the male and the female as soon as possible in order to reduce the labor required for complicated culturing. An example of a method for distinguishing the male and the female of ES cells is a method which involves amplifying and detecting by the PCR method a gene in a sex-determining region on Y chromosome. While the number of cells conventionally required for karyotyping is approximately 106, when this method is used the number of ES cells needed is about 1 colony (approximately 50 cells). Thus, the primary selection of ES cells in the early period of culturing can be performed by distinguishing the male and the female, and the early selection of male cells enabled, so that the labor required for culturing in an early period can be greatly reduced.

Furthermore, the secondary selection can be performed by, for example, confirmation or the like of the number of chromosomes by a G-banding method. As for the chromosome number of the obtained ES cells, 100% of the normal number is desirable. When this is difficult because of physical procedures or the like used for establishment, preferably, the gene of ES cell is knocked out, and then the gene is cloned again into a normal cell (for example, in a mouse, a cell wherein the chromosome number is 2n=40). The embryonic stem cell line obtained in this way usually has good proliferation ability, but easily loses its capability to contribute ontogeny. Hence, it is necessary to carefully perform subculturing. For example, a method used herein involves culturing the cells on appropriate feeder cells, such as STO fibroblasts, in the presence of LIF (1-10000 U/ml) within a carbon dioxide gas incubator (preferably, conditions of 5% carbon dioxide gas and 95% air, or 5% oxygen, 5% carbon dioxide gas and 90% air) at approximately 37° C., and when subculturing is performed, for example, obtaining single cells by treatment with trypsin/EDTA solution (normally, 0.001 to 0.5% trypsin/0.1 to 5 mM EDTA, or preferably, approximately 0.1% trypsin/1 mM EDTA), and then inoculating the cells on newly prepared feeder cells. Such subculturing is usually performed every 1 to 3 days. At this time, the cells are observed, and when morphologically abnormal cells are found, the cultured cells are preferably discarded. ES cells can be differentiated into various types of cells, such as a parietal muscle, visceral muscle, and cardiac muscle by monolayer culturing to reach a high density or by suspension culturing to form a cell cluster under appropriate conditions (Nature, 292; 154, 1981, Proc. Natl. Acad. Sci. USA, 78; 7634, 1981, J. Embryol. Exp. Molphol., 87; 27, 1985). The cells deficient in the expression of the polynucleotide of the present invention obtained by differentiating the ES cells of the present invention are useful in in vitro cell biological examination of the polynucleotide of the present invention. The non-human mammal deficient in the expression of the polynucleotide of the present invention can be distinguished from a normal animal by measuring the mRNA level of the animal by any known method and then comparing the expression levels indirectly. As the nonhuman mammal, animals similar to those described above are used.

Non-human mammals deficient in expression of the polynucleotide of the present invention can be obtained by, for example, introducing a targeting vector prepared as described above into a mouse embryonic stem cell or a mouse egg cell to inactivate the polynucleotide of the present invention, and causing homologous recombination, by which the polynucleotide sequence having the inactivated polynucleotide of the present invention is substituted with the polynucleotide of the present invention by gene homologous recombination on the chromosome of the mouse embryonic cell or the mouse egg cell, thus knocking out the polynucleotide of the present invention. The cells having the knockout polynucleotide of the present invention can be determined by Southern hybridization analysis using as a probe the polynucleotide sequence on or in the vicinity of the polynucleotide of the present invention, or analysis by the PCR method using as primers the polynucleotide sequence on the targeting vector and the polynucleotide sequence in the neighboring region other than the polynucleotide of the present invention derived from the mouse used for the targeting vector. When the embryonic stem cells of a non-human mammal are used, a cell line having the polynucleotide of the present invention inactivated by gene homologous recombination is cloned, the cells are injected into non-human mammal embryos at an appropriate timing, for example, at 8-cell stage, or blastocysts, and then the prepared chimeric embryos are transplanted into the uterus of the pseudo-pregnant non-human mammal. The thus produced animal is a chimeric animal consisting of both cells having the locus of the normal polynucleotide of the present invention, and cells having the locus of the artificially mutated polynucleotide of the present invention. When a part of the germ cells of the chimeric animal has the locus of the mutated polynucleotide of the present invention, individuals, in which all the tissues are composed of cells having the locus of the artificially mutated polynucleotide of the present invention can be obtained by selecting, for example, by coat color determination, from a group of individuals obtained by crossing such the chimeric individuals with normal individuals. The thus obtained individuals are normally individuals deficient in the hetero expression of the polypeptide of the present invention. When the individuals deficient in the hetero expression of the polypeptide of the present invention are crossed to each other, individuals deficient in the homo expression of the polypeptide of the present invention can be obtained from the progeny. When egg cells are used, for example, a polynucleotide solution is injected by a microinjection method into an egg cell nucleus so as to introduce a targeting vector within a chromosome, so that a transgenic non-human mammal having the targeting vector introduced therein can be obtained. Compared to these transgenic non-human mammals, those having the locus of DNA of the present invention mutated by gene homologous recombination can be obtained by selection.

The thus obtained individuals having the knockout polynucleotide of the present invention can be successively bred under normal breeding environment after confirming that the animal individuals obtained by crossing also have the knockout polynucleotide. Furthermore, the germ line may be obtained and maintained according to standard methods. That is, a homozygote animal having the inactivated polynucleotide on both homologous chromosomes can be obtained by crossing the female and male animals having the inactivated polynucleotide. By crossing the male with the female of the heterozygote animals, homozygote and heterozygote animals having the inactivated polynucleotide are produced, and then bred successively. The embryonic cells of the non-human mammal wherein the polynucleotide of the present invention is inactivated are very useful in producing non-human mammals deficient in the expression of the polynucleotide of the present invention. In addition, because the non-human mammal deficient in the expression of the polynucleotide of the present invention lacks various biological activities that can be induced by the polypeptide of the present invention, the non-human mammal can be a model for diseases caused by the inactivated biological activities that can be induced by the polypeptide of the present invention, so that the non-human mammal is useful in finding causes of and therapeutic methods against these diseases.

The present invention provides a method for screening for a compound or a salt thereof that promotes or inhibits the activity of the promoter for the polynucleotide of the present invention, which comprises bringing a non-human mammal deficient in the expression of the polynucleotide of the present invention or cells deficient in the expression of the polynucleotide into contact with a test compound, and then detecting the expression of a reporter gene. For example, in this screening method, as a non-human mammal deficient in the expression of the polynucleotide of the present invention, among the above non-human mammals deficient in the expression of the polynucleotide of the present invention, mammals wherein the polynucleotide of the present invention is inactivated by introducing a reporter gene, and the reporter gene can be expressed under regulation by a promoter for the polynucleotide of the present invention are used. As the reporter gene, genes similar to those described above are used, and β-galactosidase gene (lacZ), soluble alkaline phosphatase gene, luciferase gene, or the like is preferred. In the non-human mammal deficient in the expression of the polynucleotide of the present invention and cells deficient in the expression of the polynucleotide of the present invention, wherein the polynucleotide of the present invention is substituted with a reporter gene, the reporter gene is present under the control of a promoter for the polynucleotide of the present invention, so that promoter activity can be detected by tracing the expression of a substance encoded by the reporter gene.

For example, when a part of the genomic DNA region encoding the polypeptide of the present invention is substituted with a β-galactosidase gene (lacZ) derived from Escherichia coli, in a tissue, wherein the polypeptide of the present invention is originally expressed, β-galactosidase is expressed instead of the polypeptide of the present invention. Therefore, the in vivo expression state within an animal of the polypeptide of the present invention can be conveniently observed by staining using a reagent that can be a substrate of β-galactosidase, such as 5-bromo-4-chloro-3-indolyl-β-galactopyranoside (X-gal). Specifically, a mouse deficient in the polypeptide of the present invention or a tissue section thereof is immobilized using glutaraldehyde or the like, washed with phosphate buffered saline, and then allowed to react with a stain containing X-gal at room temperature or at around 37° C. for about 30 minutes or 1 hour. Next, the tissue sample is washed with a 1 mM EDTA/PBS solution to stop P-galactosidase reaction, and then color development may be observed. In addition, according to a standard method, mRNA encoding lacZ may be detected.

The compound or the salt thereof obtained using the above screening method is, for example, a compound selected from a peptide, protein, non-peptide compound, synthetic compound, fermentation product, cell extract, plant extract, animal tissue extract, plasma, and the like, and inhibits or promotes the promoter activity for the polypeptide of the present invention. The compound obtained by the screening method may form a salt. As a salt of the compound, a salt formed with a physiologically acceptable acid (for example, inorganic acid) or a base (for example, organic acid) is used, and in particular, physiologically acceptable acid addition salt is preferred. As such a salt, for example, a salt formed with inorganic acid (for example, hydrochloric acid, phosphoric acid, hydrobromic acid, and sulfuric acid) or a salt formed with organic acid (for example, acetic acid, formic acid, propionic acid, fumaric acid, maleic acid, succinic acid, tartaric acid, citric acid, malic acid, oxalate, benzoic acid, methanesulfonic acid, and benzenesulfonic acid) is used. The compound or the salt thereof that inhibits the promoter activity for the polynucleotide of the present invention inhibits the expression of the polypeptide of the present invention, and thus can inhibit the function of the polypeptide. Therefore, the compound or the salt thereof is useful as a safe and low toxic pharmaceutical, such as a therapeutic and prophylactic agent against, for example, post-herpetic neuralgia, delayed post-operative pain, diabetic neuropathy, neuropathy after radiation exposure, protracted pain after blood collection and insertion of indwelling needle, pain after dismemberment, CRPS, a part of cancer pain, thalamic pain, pain after spinal cord injury, trigeminal neuralgia, glossopharyngeal neuralgia, various types of pain symptoms or symptoms of dysesthesia. Furthermore, a compound derived from the compound obtained by the above screening method can also be used similarly.

The pharmaceutical containing the compound or the salt thereof obtained by the above screening method can be produced in a manner similar to that for a pharmaceutical containing the above polypeptide or the salt thereof of the present invention. The thus obtained pharmaceutical preparation can be administered to a human or a mammal (for example, rats, mice, guinea pigs, rabbits, sheep, pigs, cattle, horses, cats, dogs, and monkeys). The dose of the compound or the salt differs depending on the disease to be treated, the subject to be administered, and the route of administration. For example, a compound that promotes the promoter activity for the polynucleotide of the present invention is orally administered to treat pain, generally the compound is administered to an adult (suppose 60 kg in weight) in an amount of approximately 0.1 to 100 mg per day, preferably, approximately 1.0 to 50 mg, or more preferably approximately 1.0 to 20 mg. When administered parenterally, a single dose of the compound differs depending on the subject to be administered, the disease to be treated and the like. For example, when a compound that promotes the promoter activity for the polynucleotide of the present invention is administered to treat pain in the form of an injection to a normal adult (suppose the weight is 60 kg), it is convenient to administer by intravenous injection of approximately 0.01 to 30 mg, preferably approximately 0.1 to 20 mg, or more preferably approximately 0.1 to 10 mg of the compound per day. The compound that promotes the promoter activity for the polynucleotide of the present invention can be administered in a manner similar to the above.

In addition to the compound that regulates the promoter activity for the polynucleotide of the present invention, examples of a means to regulate the expression of the polynucleotide of the present invention include a method using an antisense molecule and a ribozyme molecule that block the translation of a gene, and a method that blocks transcription by the formation of triple helices using a region which is complementary to the 5′ region of a gene. These methods can finally suppress in vivo the function of the polypeptide of the present invention or the polynucleotide of the present invention, so that they can be used as, for example, therapeutic and prophylactic agents against diseases caused by the overexpression of the polypeptide of the present invention and the like. When the antisense molecule is used as the above therapeutic and prophylactic agent, it can be used in a manner similar to that for therapeutic and prophylactic agents (described later) against various diseases containing the polynucleotide of the present invention. For example, when the antisense molecule is used, the antisense molecule alone, or the antisense molecule after insertion into an appropriate vector, such as a retrovirus vector, adenovirus vector, or adenovirus-associated virus vector, can be administered orally or parenterally to a human or a mammal according to conventional means. Furthermore, the antisense molecule can be used as a diagnostic oligonucleotide probe to diagnose the pain state by examining the presence or the expression state of the polynucleotide of the present invention in tissues or cells.

Use of the Polynucleotide of the Present Invention and the Like

Hereinafter, the applications of the polynucleotide of the present invention, a polypeptide encoded by the polynucleotide of the present invention or a salt thereof (hereinafter, may also be abbreviated as “the polypeptide of the present invention”), a partial peptide of the polypeptide encoded by the polynucleotide of the present invention, or a salt thereof (hereinafter, may also be abbreviated as “the partial peptide of the present invention”), antibody against the polypeptide of the present invention, the partial peptide or salts thereof (hereinafter, may also be abbreviated as “the antibody of the present invention”) are described.

Respective genes identified in this specification or the polynucleotide of the present invention can be used as a reagent in many various methods. Descriptions given below should be regarded as illustrative, and in which known techniques are utilized.

For example, when the polynucleotide of the present invention is used as a probe, abnormalities (abnormality of the genes) in DNA or mRNA encoding the polypeptide or the partial peptide of the present invention in a human or a warm-blooded animal (for example, rats, mice, guinea pigs, rabbits, sheep, pigs, cattle, horses, cats, dogs, monkeys, chimpanzees, and birds) can be detected. Thus, for example, the polynucleotide of the present invention is useful as an agent for gene diagnosis to diagnose damage, mutations, or a decrease in the expression of the DNA or mRNA, and an increase or overexpression of the DNA or mRNA. The above gene diagnosis using the polynucleotide of the present invention can be performed by the methods known per se, for example, Northern hybridization, PCR-SSCP (Genomics, 5; 874, 1989, Proc. Natl. Acad. Sci. USA, 86; 2766, 1989), and DNA microarray. For example, when overexpression is detected by Northern hybridization or DNA microarray, or when a mutation is detected in the target gene according to the present invention by the PCR-SSCP method or DNA microarray, it is possible to diagnose diathesis of pain, or a pain disorder against which existing analgesics cannot easily exert its effect, or the like. Therefore, the polynucleotide of the present invention can be used in a diagnostic monitoring method for a patient to be clinically evaluated upon pain therapy, a method for monitoring the clinical effect of a compound, and a method for identifying a patient who has diathesis of pain. Furthermore, the polynucleotide of the present invention can be used as a marker or a molecular weight marker to identify a chromosome, or for selecting or preparing an oligomer to adhere to a “gene chip” or other supports.

Respective gene products identified in this specification or the polypeptide of the present invention can be used as a reagent in many various methods. Descriptions given below should be regarded as illustrative, and in which known techniques are utilized.

The polypeptide of the present invention can be used as a tissue or cell marker, because it can be expressed specifically to a tissue or a cell. That is, it is useful as a marker for detecting differentiation of tissues and cells, and pathologic conditions, such as pain. Further, it can also be used for obtaining a corresponding receptor, binding protein, binding DNA and the like. Furthermore, it can also be utilized as a panel for high throughput screening known per se to examine biological activity. Further, the three-dimensional structure of the polypeptide of the present invention can be determined by NMR or X-ray analysis. The three-dimensional structure can provide information to be used to design a compound that acts with the polypeptide of the present invention. Specifically, a compound that interacts, such as by binding, in silico, with the polypeptide of the present invention can be designed and screened.

When there is an abnormality or deficiency in the polypeptide of the present invention, or in the polynucleotide of the present invention, or when expression levels in the same are decreased or enhanced abnormally, pain symptoms can be recognized. Therefore, the polypeptide of the present invention can be used as a pharmaceutical, such as a therapeutic and prophylactic agent against, for example, post-herpetic neuralgia, delayed post-operative pain, diabetic neuropathy, neuropathy after radiation exposure, protracted pain after blood collection and insertion of indwelling needle, pain after dismemberment, CRPS, a part of cancer pain, thalamic pain, pain after spinal cord injury, trigeminal neuralgia, glossopharyngeal neuralgia, various types of pain symptoms or symptoms of dysesthesia. For example, when a patient is in a pathological condition wherein the polypeptide and the like of the present invention increase in vivo, so that transmission in the cells or nerve is enhanced beyond a required level, a compound that reduces the expression of the polypeptide of the present invention, the antibody of the present invention or the like is administered so as to suppress the biological activity of the polypeptide of the present invention, so that the patient can be treated by suppressing quantitatively and qualitatively the polypeptide of the present invention to cause the polypeptide to be able to exert the functional role normally. Conversely, for example, when there is a patient whose pathological condition is formed because the polypeptide and the like of the present invention are decreased in vivo to cause insufficient transmission in cells or nerve, the symptoms can be alleviated by (a) administering the polynucleotide of the present invention to the patient to cause the polypeptide of the present invention to be expressed in vivo, (b) inserting the polynucleotide of the present invention into cells to cause the polypeptide of the present invention to be expressed, and then transplanting the cells into the patient, or (c) administering the polypeptide of the present invention to the patient to enhance quantitatively and qualitatively the polypeptide of the present invention in the patient, so as to cause the polypeptide to be able to exert the functional role normally.

When the polynucleotide of the present invention is used as the above therapeutic and prophylactic agent, the polynucleotide alone, or the polynucleotide after it is inserted into an appropriate vector, such as a retrovirus vector, adenovirus vector, or adenovirus associated virus vector, can be administered to a human or a warm-blooded animal according to conventional means. The polynucleotide of the present invention can be formulated intact or can be formulated with a physiologically acceptable carrier, such as an adjuvant for promoting ingestion, and then administered via a gene gun or a catheter, such as a hydrogel catheter. When the protein of the present invention is used as the above therapeutic and prophylactic agent, the protein purified to at least 90%, preferably 95% or more, more preferably 98% or more, or further preferably 99% or more is preferably used.

The polypeptide of the present invention can be used orally as, for example, a tablet, which is sugar-coated, if necessary, capsule, elixir, microcapsule, or parenterally in the form of an injection, such as an sterile solution with water or a pharmaceutically-acceptable solution other than water, or suspension. For example, the protein of the present invention can be produced as a pharmaceutical preparation by admixing it in a unit dosage form as required by generally recognized formulation practice, with a physiologically acceptable carrier, flavor agent, excipient, vehicle, antiseptic, stabilizer, binder or the like. The pharmaceutical preparation is produced so that it contains an active ingredient in an appropriate amount within a range as indicated. Examples of additives that can be admixed with a tablet, capsule or the like include a binder such as gelatine, corn starch, gum tragacanth, or gum arabic; an excipient such as crystalline cellulose; a swelling agent such as corn starch, gelatine, or alginic acid; a lubricant such as magnesium stearate; a sweetening agent such as sucrose, lactose or saccharin; and a flavor agent, such as peppermint, akamono (Gautheria ovatifolia) oil or cherry. When a unit form for dispensing is a capsule, it can contain a liquid carrier, such as fat and oil, in addition to the above types of materials. Sterile compositions for injection can be prescribed in accordance with normal formulation practice such as dissolving or suspending an active substance in a vehicle such as water for injection, natural plant oil such as sesame oil or coconut oil, or the like. Examples of an aqueous solution for injection include physiological saline, an isotonic solution containing glucose and other adjuvants (for example, D-sorbitol, D-mannitol, and sodium chloride), and the like. The aqueous solution may be used together with an appropriate solubilizer, for example, alcohol (for example, ethanol), polyalcohol (for example, propylene glycol or polyethylene glycol), or a nonionic surfactant (for example, polysorbate 80™ or HCO-50). Examples of an oil solution include sesame oil, and soybean oil, and the oil solution may be used together with a solubilizer, such as benzyl benzoate or benzyl alcohol. Further, it may also be compounded with a buffer (for example, phosphate buffer or sodium acetate buffer), a soothing agent (for example, benzalkonium chloride or procaine hydrochloride), a stabilizer (for example, human serum albumin or polyethylene glycol), a preservative (for example, benzyl alcohol or phenol), and an oxidation inhibitor. A prepared parenteral solution is normally filled in an appropriate ampule. The vector having the polynucleotide of the present invention inserted therein is formulated in a manner similar to the above, and is normally used parenterally.

The pharmaceutical preparation obtained in this way is safe and has low toxicity, so that it can be administered to, for example, a human or a warm-blooded animal (for example, rats, mice, guinea pigs, rabbits, sheep, pigs, cattle, horses, cats, dogs, monkeys, chimpanzees, and birds). The dose of the polypeptide of the present invention differs depending on the disease to be treated, the subject to be administered, the route of administration and the like. For example, when the polypeptide of the present invention is orally administered to treat pain, in general, approximately 1 mg to 1000 mg, preferably approximately 10 to 500 mg, or more preferably approximately 10 to 200 mg of the polypeptide is administered to an adult (suppose 60 kg in weight) per day. When the polypeptide is administered parenterally, a single dose of the polypeptide differs depending on the subject to be administered, the disease to be treated, and the like. For example, when the polypeptide of the present invention is administered to treat pain in the form of an injection to an adult (suppose 60 kg in weight), it is convenient to administer by injection of approximately 1 to 1000 mg, preferably, approximately 1 to 200 mg, or more preferably approximately 10 to 100 mg of the protein per day to the affected part. In the case of other animals, the amount of the polypeptide converted from an amount per 60 kg can be administered.

The compound or a salt thereof that inhibits the function of the polypeptide of the present invention can be used as a pharmaceutical, such as a therapeutic and prophylactic agent against, for example, post-herpetic neuralgia, delayed post-operative pain, diabetic neuropathy, neuropathy after radiation exposure, protracted pain after blood collection and insertion of indwelling needle, pain after dismemberment, CRPS, a part of cancer pain, thalamic pain, pain after spinal cord injury, trigeminal neuralgia, glossopharyngeal neuralgia, various types of pain symptoms or symptoms of dysesthesia. On the other hand, the compound or a salt thereof that enhances the function of the polypeptide of the present invention can be used as a pharmaceutical, such as a therapeutic and prophylactic agent against a disease caused by a decrease or deficiency in the production of the polypeptide of the present invention.

The polypeptide of the present invention is useful as a reagent for screening for the compound or a salt thereof that inhibits or promotes the function of the polypeptide of the present invention. That is, the present invention provides a method which uses the polypeptide of the present invention, a partial peptide thereof, or salts thereof, for screening for a compound or a salt thereof that inhibits the function of the polypeptide of the present invention, a partial peptide thereof, or a salt thereof (hereinafter, may also be abbreviated as “an inhibitor”), or a compound that promotes the function of the polypeptide of the present invention, a partial peptide thereof, or a salt thereof (hereinafter, may also be abbreviated as “a promoter”). A screening kit of the present invention contains the polypeptide of the present invention, a partial peptide thereof or a salt thereof.

A cell or a transformant that contains and expresses a target gene sequence encoding the target gene product of the present invention and shows a cell phenotype relating to pain can also be utilized for identifying a compound that acts on the polypeptide of the present invention or a partial peptide thereof, in addition to being used to produce the polypeptide or the partial peptide thereof of the present invention. Examples of such a cell include those transformed with a non-recombinant neuroglioma cell line or a general mammal cell line, such as HeLa cells, COS cells, or CHO cells. Another example of such a cell is a recombinant transgenic cell line. For example, the above transgenic animal can be used as a cell culture model of pain symptoms, and is used for producing a cell line that contains one or more cell types involved in pain. A primary culture product obtained from the transgenic animal of the present invention can be utilized, but the production of a continuous cell line is preferred. An example of a method used for obtaining a continuous cell line from the transgenic animal is described in Mol. Cell. Biol., 5; 642 (1985). Alternatively, cells of a cell type that is known to be involved in pain may be transformed with a polynucleotide that can increase or decrease the expression level of a target gene within a cell. For example, a target gene may be introduced and over-expressed within the genome of a target cell. Alternatively, when an endogenous target gene is present, a target gene is introduced into the above genome so as to cause overexpression of the target gene, or to disrupt the endogenous target gene, so that the endogenous target gene may be under-expressed or the expression thereof may be inactivated.

To achieve overexpression of a target gene, the coding region of the target gene sequence is ligated to a regulatory sequence that can direct the expression of the gene in a target cell type. Such a regulatory sequence is known by a person skilled in the art, and can be utilized without excessive experimentation. A recombination method for the expression of a target gene is included in the above description about a method which involves culturing a transformant containing a polynucleotide encoding a polypeptide or a partial peptide to produce the polypeptide or the partial peptide of the present invention. To achieve underexpression of an endogenous target gene, the isolated target gene is produced so that when it is re-introduced into the genome of a target cell type, an allele of the endogenous target gene is inactivated. Preferably, a target gene sequence to be produced herein is introduced by gene targeting, so that an endogenous target sequence is disrupted when the produced target gene sequence is incorporated into the genome of a cell. A transformation method of a host cell using a target gene is as described above. Cells treated with a compound or cells transformed with a target gene can be examined for pain-related phenotypes. Transformed cells are assessed using as indicators the presence or absence of a recombinant target gene sequence, expression and accumulation of mRNA of a target gene, and production of a recombinant target gene product. When decreased expression of a target gene is preferred, it is verified using a standard method whether or not decreases in the expression of an endogenous target gene and/or the production of the target gene product is achieved. The above-described cells or transformants containing and expressing a target gene sequence that encodes a target gene product and shows pain-related cell phenotypes, provide a method for screening for a compound that acts on the polypeptide or a partial peptide thereof of the present invention, which is characterized by treating the transformant with a compound.

The compound or a salt thereof that is obtained using the screening method or the screening kit of the present invention is a compound that is selected from, for example, a peptide, protein, nonpeptidic compound, synthetic compound, fermentation product, cell extract, plant extract, animal tissue extract, and plasma, includes an antibody and the like against the polypeptide or the partial peptide of the present invention, and inhibits or promotes the function of the polypeptide of the present invention. As a salt of the compound, a salt similar to the above salt of the polypeptide of the present invention is used.

When the compound obtained using the screening method or the screening kit of the present invention is used as the above therapeutic and prophylactic agent, it can be used according to conventional means. For example, in a manner similar to that of the above pharmaceutical containing the protein of the present invention, the compound can be used orally as, for example, a tablet, which is sugar-coated if necessary, capsule, elixir, or microcapsule, or can be used parenterally in the form of an injection, such as an sterile solution with water or a pharmaceutically-acceptable solution other than water, or suspension. The thus obtained pharmaceutical preparation can be administered to a human or a warm-blooded animal. The dose of the compound or a salt thereof differs depending on the action, the disease to be treated, the subject to be administered, the route of administration, and the like. Generally, for example, approximately 0.1 to 100 mg, preferably approximately 1.0 to 50 mg, or more preferably approximately 1.0 to 20 mg of the compound that inhibits the function of the polypeptide of the present invention is administered orally to an adult (suppose 60 kg in weight) to treat pain. In the case of parenteral administration, a single dose of the compound differs depending on the subject to be administered, the disease to be treated, and the like. For example, when the compound that inhibits the function of the polypeptide of the present invention is administered, in the form of an injection, to an adult (suppose 60 kg in weight) to treat pain, it is convenient to administer per day by intravenous injection approximately 0.01 to 30 mg, preferably approximately 0.1 to 20 mg, or more preferably approximately 0.1 to 10 mg of the compound.

The antibody of the present invention can be used for quantitatively determining the polypeptide of the present invention in a sample solution, particularly by the sandwich immunoassay. That is, the present invention provides (i) a method for quantitatively determining the polypeptide of the present invention in a sample solution, which comprises causing competitive reaction among the antibody of the present invention, a sample solution and the labeled protein of the present invention, and then measuring the proportion of the labeled polypeptide of the present invention bound to the antibody, and (ii) a method for quantitatively determining the polypeptide of the present invention in a sample solution, which comprises causing simultaneous or successive reaction among a sample solution, the antibody of the present invention insolubilized on a carrier, and another labeled antibody of the present invention, and then measuring the activity of a labeling agent on the insolubilized carrier.

Further, the polypeptide of the present invention can be quantitatively determined using the monoclonal antibody for the polypeptide of the present invention (hereinafter, may be referred to as “the monoclonal antibody of the present invention”), while detection using histological staining or the like can also be performed. For such quantitative determination and detection, an antibody molecule itself may be used, and the fraction of an antibody molecule, F(ab′)₂, Fab′, or Fab may also be used. The quantitative determination method of the protein of the present invention using the antibody of the present invention is not specifically limited, and any measurement method may be used, as long as it comprises detecting by a chemical or physical means the amount of antibody corresponding to the amount of antigen (for example, protein amount), the amount of antigen, or the amount of antibody-antigen complex in a test solution, and calculating based on a standard curve prepared using a standard solution containing a known amount of antigen. For example, nephelometry, a competitive method, immunometric method, and sandwich method are preferably used. In terms of sensitivity and specificity, the use of the sandwich method described later is particularly preferred. Examples of a labeling agent to be used in a measurement method using a labeling substance include radioactive isotopes, enzymes, fluorescent substances, and luminous substances. As a radioactive isotope, for example, [¹²⁵I], [¹³¹I], [³H], [¹⁴C] and the like are used. As the above enzyme, a stable enzyme having a high specific activity is preferred. Examples of such an enzyme that is used herein include β-galactosidase, β-glucosidase, alkaline phosphatase, peroxidase, and malate dehydrogenase. As a fluorescent substance, for example, fluorescamine or fluorescein isothiocyanate is used. As a luminous substance, for example, luminol, luminol derivative, luciferin, or lucigenin is used. Further, a biotin-avidin system may also be used for binding of an antibody or antigen with a labeling agent.

Antigens or antibodies may be insolubilized by physical adsorption, or by a method which uses chemical binding that is normally used for insolubilizing and immobilizing protein, enzyme or the like. Examples of a carrier include insoluble polysaccharides, such as agarose, dextran, and cellulose, synthetic resins, such as polystyrene, polyacryl amide, and silicon, or glass. In the sandwich method, a sample solution is allowed to react (primary reaction) with the insolubilized monoclonal antibody of the present invention, another labeled monoclonal antibody of the present invention is then allowed to react (secondary reaction), and the activity of the labeling agent on the insolubilized carrier is measured, so that the amount of the protein of the present invention in the sample solution can be quantitatively determined. The primary reaction and secondary reaction may be performed in a reverse order, simultaneously, or at different times. Labeling and insolubilization may be according to the above methods. Further, in immunoassay by the sandwich method, the number of types of antibodies to be used as antibodies for immobilization or for labeling is not necessarily just one type, and a mixture of two types or more antibodies may be used to improve measurement sensitivity and the like. In the method for measuring the polypeptide of the present invention using the sandwich method of the present invention, the monoclonal antibodies of the present invention to be used for the primary and secondary reactions preferably differ in the site to which the polypeptide of the present invention is bound. Specifically, regarding the antibodies to be used in the primary and the secondary reactions, for example, when antibodies to be used in the secondary reaction recognize the C-terminal region of the protein of the present invention, antibodies to be used in the primary reaction preferably recognize a region other than the C-terminal region, for example, the N-terminal region.

The monoclonal antibody of the present invention can be used for a measurement system other than the sandwich method, for example, for a competitive method, immunometric method, or nephelometry. In the competitive method, after the antigens in a sample solution and labeled antigens are allowed to react competitively with antibodies, unreacted labeled antigens (F) are separated from labeled antigens (B) bound to antibodies (B/F separation), the amount of the label of either B or F is measured, and then the amount of the antigens in the sample solution is quantitatively determined. In this reaction method, a liquid phase method using soluble antibodies as antibodies, polyethylene glycol for B/F separation, and second antibodies or the like for the above antibodies; and a solid phase method using immobilized antibodies as the first antibody, or using soluble antibodies as the first antibody and immobilized antibodies as the second antibody, are used. In the immunometric method, after the antigens in a sample solution and immobilized antigens are allowed to competitively react with a certain amount of labeled antibodies, the solid phase is separated from the liquid phase; or the antigens in a sample solution are allowed to react with an excessive amount of labeled antibodies, immobilized antigens are then added so as to allow unreacted labeled antibodies to bind to the solid phase, and then the solid phase is separated from the liquid phase. Next, the amount of the label of either solid or liquid phase is measured, so that the amount of the antigens in the sample solution is quantitatively determined. Moreover, in nephelometry, the amount of insoluble precipitate resulting from antigen-antibody reaction within gel or in a solution is measured. Even when the amount of antigen in a sample solution is very small, and only a small amount of precipitate is obtained, for example, laser nephelometry utilizing laser scattering is preferably used.

To apply these immunoassays respectively to the quantitative determination method of the present invention, the setting of unusual conditions and procedures, such as operations, is not required. The measurement system of the polypeptide of the present invention may be constructed by containing usual conditions and procedures for each method with usual technical consideration by a person skilled in the art. Regarding details about these general technical measures, reviews, reference books and the like can be referred to. For example, “Radioimmunoassay 2” (ed., Hiroshi IRIE, KODANSHA, issued 1979), “Enzyme Immunoassay” (2^{nd} ed, Eiji ISHIKAWA et al.), (Igaku-Shoin, issued 1982), “Enzyme Immunoassay” (3^{rd} ed., Eiji ISHIKAWA et al.), (Igaku-Shoin, issued 1987), “Methods in ENZYMOLOGY,” vol. 70 (Immunochemical Techniques) (Part A), “Methods in ENZYMOLOGY,” vol. 73 (Immunochemical Techniques) (Part B), “Methods in ENZYMOLOGY” vol. 74 (Immunochemical Techniques) (Part C), “Methods in ENZYMOLOGY,” vol. 84 (Immunochemical Techniques) (Part D), “Methods in ENZYMOLOGY,” vol. 92 (Immunochemical Techniques) (Part E), and “Methods in ENZYMOLOGY,” vol. 121 (Immunochemical Techniques) (Part I) (all issued by Academic Press) and the like can be referred to. As described above, the polypeptide of the present invention can be quantitatively measured with good sensitivity using the antibody of the present invention.

When an increase is detected in the concentration of the polypeptide of the present invention by quantitative determination of the concentration of the polypeptide of the present invention using the antibody of the present invention, it enables diagnosis of the suspicion of having, or the high probability of having in the future, various pain symptoms or the symptoms of dysaesthesia, such as post-herpetic neuralgia, delayed post-operative pain, diabetic neuropathy, post-radiation irradiation neuropathy, protracted pain after blood collection and protracted pain after insertion of an indwelling needle, pain after dismemberment, CRPS, and a part of cancer pain, thalamic pain, pain after spinal cord injury, trigeminal pain, and glossopharyngeal neuralgia. Moreover, from the degree of an increase in the concentration of the polypeptide of the present invention, the degree of pain itself can be diagnosed and assessed. Therefore, the antibody of the present invention can be used in a diagnostic method for monitoring a patient to be clinically evaluated upon pain treatment, a method for clinically monitoring the efficiency of a compound, and a method for identifying a patient having diathesis of pain. Further, the antibody of the present invention can be used for detecting the polypeptide of the present invention existing in a specimen such as body fluid and tissue. Furthermore, the antibody can also be used for, for example, preparing an antibody column that is used for purifying the polypeptide of the present invention, detecting the polypeptide of the present invention in each fraction upon purification, and analyzing the behavior of the polypeptide of the present invention within a cell to be tested.

The antibody of the present invention, which has an effect of neutralizing the activity of the polypeptide of the present invention can be used as, for example, a pharmaceutical, such as a therapeutic and prophylactic agent against a disease caused by the overexpression of the polypeptide or the like of the present invention. The therapeutic and prophylactic agent against the above disease containing the antibody of the present invention can be safely administered orally or parenterally as an intact solution, or as a pharmaceutical composition in an appropriate dosage form to a human or a mammal (for example, rats, rabbits, sheep, pigs, cattle, cats, dogs and monkeys). The dose differs depending on the subject to be administered, the disease to be treated, the symptoms, and the route of administration. For example, it is convenient to administer, by intravenous injection, normally, approximately 0.01 to 20 mg/kg weight, preferably approximately 0.1 to 10 mg/kg weight, or more preferably approximately 0.1 to 5 mg/kg weight of a single dose of the antibody of the present invention about 1 to 5 times a day, or preferably about 1 to 3 times a day. In the case of other parenteral and oral administrations, the amount of the antibody according to the above dose may be administered. When the symptom is particularly severe, the dose may be increased depending on the symptom. The antibody of the present invention can be administered by itself or as a suitable pharmaceutical composition. The pharmaceutical composition used in the above administration contains the antibody of the present invention, a pharmacologically acceptable carrier, diluent or excipient. Such composition is provided in a dosage form appropriate for oral or parenteral administration. That is, for example, examples of a composition for oral administration include a composition in a solid or liquid dosage form, specifically, a tablet (including a sugar-coated tablet and film coating tablet), pill, granule, powder, capsule (including a soft capsule), syrup, emulsion, and suspension. Such a composition is produced by a method known per se, and contains a carrier, diluent or excipient that is normally used in the field of pharmaceutical preparation. For example, as a carrier and excipient for a tablet, lactose, starch, sucrose, magnesium stearate and the like are used.

As a composition for parenteral administration, for example, an injection, suppository and the like are used. The injection encompasses dosage forms such as an intravenous injection, subcutaneous injection, intradermal injection, intramuscular injection, intravenous drip injection, and the like. Such an injection can be prepared according to a method known per se, for example by dissolving, suspending or emulsifying the above antibody or a salt thereof in a sterile aqueous or oil solution that is normally used for an injection. As an aqueous solution for an injection, for example, a physiological saline, an isotonic solution containing glucose or other adjuvants, or the like are used. The solution for injection may also be used with an appropriate solubilizer such as alcohol (for example, ethanol), polyalcohol (for example, propylene glycol or polyethylene glycol), or a nonionic surfactant (for example, polysorbate 80 or HCO-50). As an oil solution, for example, sesame oil or soybean oil is used, and the solution may be used with a solubilizer such as benzyl benzoate or benzyl alcohol. The prepared injection is normally filled in an appropriate ampule. A suppository used for rectal administration is prepared by mixing the above antibody or a salt thereof with a normal base for suppository. It is convenient to prepare the above oral or parenteral pharmaceutical composition into a dosage unit form adequate for the dose of an active ingredient. As such a dosage unit form, a tablet, pill, capsule, injection (ampule), suppository, and the like are exemplified. Normally, 5 to 500 mg, in particular for an injection, 5 to 100 mg, and in another dosage form, 10 to 250 mg of the above antibody is preferably contained per dosage unit form. In addition, each of the above described compositions may contain other active ingredients, as long as the formulation with the above antibody does not cause unfavorable interaction.

In this specification, when nucleotides, amino acids, and the like are indicated by abbreviation and drawings, the indication is based on the abbreviations of IUPAC-IUB (Commission on Biochemical Nomenclature) or abbreviations common in the art, and examples are shown below. Further, when an optical isomer can be present for amino acid, it indicates an L-form, unless otherwise specified. DNA: deoxyribonucleic acid, cDNA: complementary deoxyribonucleic acid, A: adenine, T: thymine, G: guanine, C: cytosine, RNA: ribonucleic acid, mRNA: messenger ribonucleic acid, dATP: deoxyadenosine triphosphate, dTTP: deoxythymidine triphosphate, dGTP: deoxyguanosine triphosphate, dCTP: deoxycytidine triphosphate, ATP: adenosine triphosphate, EDTA: ethylene diamine tetraacetic acid, SDS: sodium dodecyl sulfate, Gly: glycine, Ala: alanine, Val: valine, Leu: leucine, Ile: isoleucine, Ser: serine, Thr: threonine, Cys: cysteine, Met: methionine, Glu: glutamic acid, Asp: aspartic acid, lys: lysine, Arg: arginine, His: histidine, Phe: phenylalanine, Tyr: tyrosine, Trp: tryptophan, Pro: proline, Asn: asparagine, Gln: glutamine, pGlu: pyroglutamic acid.

Substituents, protecting groups and reagents that are frequently used in this specification are denoted with the following symbols.

Me: methyl group, Et: ethyl group, Bu: butyl group, Ph: phenyl group, TC: thiazolidine-4(R)-carboxyamide group, Tos: p-toluenesulfonyl, CHO: formyl, Bzl: benzil, C12-Bzl: 2,6-dichlorobenzyl, Bom: benzyloxymethyl, Z: benzyloxycarbonyl, Cl-Z: 2-chlorobenzyloxy carbonyl, Br-Z: 2-bromo benzyloxycarbonyl, Boc: t-butoxycarbonyl, DNP: dinitrophenyl, Trt: trityl, Bum: t-butoxymethyl, Fmoc: N-9-fluorenylmethoxycarbonyl, HOBt: 1-hydroxy benztriazole, HOOBt: 3,4-dihydro-3-hydroxy-4-oxo-1,2,3-benzotriazine, HONB: 1-hydroxy-5-norbornene-2,3-dicarboximide, DCC: N,N′-dicyclohexylcarbodiimide

In the sequence listing and the specification of the present application, the polynucleotide sequences or amino acid sequences are represented by the SEQ ID NOS as follows.

“SEQ ID NO: 1” represents the polynucleotide sequence of ch7L-7 gene (clone ID NO: C630002H18) according to the present invention.

“SEQ ID NO: 2” represents the polynucleotide sequence of ch7L-14 gene (clone ID NO: C130099K08) according to the present invention.

“SEQ ID NO: 3” represents the polynucleotide sequence of ch8L-32 gene (clone ID NO: 5930404010) according to the present invention.

“SEQ ID NO: 4” represents the polynucleotide sequence of phM-8 gene (clone ID NO: A330018G02) according to the present invention.

“SEQ ID NO: 5” represents the polynucleotide sequence of phS-3 gene (clone ID NO: 9930124N15) according to the present invention.

“SEQ ID NO: 6” represents the polynucleotide sequence of phX-6 gene (clone ID NO: B330016G20) according to the present invention.

“SEQ ID NO: 7” represents the polynucleotide sequence of a human homologous gene of ch7L-7 gene according to the present invention.

“SEQ ID NO: 8” represents the polynucleotide sequence of a human homologous gene of ch7L-14 gene according to the present invention.

“SEQ ID NO: 9” represents the polynucleotide sequence of a human homologous gene of ch8L-32 gene according to the present invention.

“SEQ ID NO: 10” represents the polynucleotide sequence of a human homologous gene of phS-3 gene according to the present invention.

“SEQ ID NO: 11” represents the polynucleotide sequence of a human homologous gene of phX-6 gene according to the present invention.

“SEQ ID NO: 12” represents the amino acid sequence of a polypeptide encoded by the polynucleotide sequence represented by SEQ ID NO: 1.

“SEQ ID NO: 13” represents the amino acid sequence of a polypeptide encoded by the polynucleotide sequence represented by SEQ ID NO: 2.

“SEQ ID NO: 14” represents the amino acid sequence of a polypeptide encoded by the polynucleotide sequence represented by SEQ ID NO: 3.

“SEQ ID NO: 15” represents the amino acid sequence of a polypeptide encoded by the polynucleotide sequence represented by SEQ ID NO: 4.

“SEQ ID NO: 16” represents the amino acid sequence of a polypeptide encoded by the polynucleotide sequence represented by SEQ ID NO: 5.

“SEQ ID NO: 17” represents the amino acid sequence of a polypeptide encoded by the polynucleotide sequence represented by SEQ ID NO: 6.

“SEQ ID NO: 18” represents the amino acid sequence of a polypeptide encoded by the polynucleotide sequence represented by SEQ ID NO: 7.

“SEQ ID NO: 19” represents the amino acid sequence of a polypeptide encoded by the polynucleotide sequence represented by SEQ ID NO: 8.

“SEQ ID NO: 20” represents the amino acid sequence of a polypeptide encoded by the polynucleotide sequence represented by SEQ ID NO: 9.

“SEQ ID NO: 21” represents the amino acid sequence of a polypeptide encoded by the polynucleotide sequence represented by SEQ ID NO: 10.

“SEQ ID NO: 22” represents the amino acid sequence of a polypeptide encoded by the polynucleotide sequence represented by SEQ ID NO: 11.

### EXAMPLE

The following examples are presented to further illustrate the present invention, and are not intended to limit the present invention. Genetic engineering procedures using Escherichia coli were performed according to the methods described in Molecular Cloning 2nd Edition (J. Sambrook et al., Cold Spring Harbor Lab. Press, 1989). In the examples described herein, 6 genes having novel sequences were identified, and it was demonstrated that these genes are differentially expressed in the condition exhibiting pain symptoms. Identification of these genes and characterization of their expression in specific disease conditions makes it possible to recognize new roles of these genes in pain. Specifically, the novel genes ch7L-7 (clone ID NO: C630002H18), ch7L-14 (clone ID NO: C130099K08), ch8L-32 (clone ID NO: 5930404010), phM-8 (clone ID NO: A330018G02), phS-3 (clone ID NO: 9930124N15), and phX-6 (clone ID NO: B330016G20) are respectively regulated differentially in individuals exhibiting hyperalgesia or symptoms of allodynia.

### Example 1

### Preparation of Neuropathic Pain Model—1 (Chung Model)

The regions from the dorsolumber to gluteal regions of 5-week-old SD male rats were shaved under pentobarbital anesthesia. The Chung model was produced according to the method of Kim and Chung (Pain, 50; 355, 1992). The shaved region was incised along the median line, and then the left paraspinal muscle was ablated from the processus at L4-S2 levels. After the removal of processus pterygoideus so as to be able to see L4-L6 spinal nerves, L5 and L6 spinal nerves were isolated, and two positions each were ligated firmly using silk suture No. 6-0. Then, the incised regions were sutured with silk suture No. 4. At an appropriate time between 1 to 12 days after operation, pain threshold was measured. The pain threshold measurements were performed by housing each rat in an acryl measurement cage with a stainless mesh bottom that was placed at a height of 20 cm (W125×D200×H200 (mm)), habituating the rats for approximately 1 hour, and then stimulating with von Fray filament the central parts of the soles of the left and the right hind legs of each rat. During stimulation or immediately after the end of stimulation, the presence or absence of avoidance reaction from the filament were examined. Avoidance reaction appeared strongly on the hind leg on the side of the ligations, and thereby the development of allodynia was confirmed.

### Example 2

### Production of Neuropathic Pain Model—2 (Shingles Pain Model)

Shingles pain models were produced according to the method described in JP Patent Application No. 2000-157602 or Pain, 86; 95, 2000. Specifically, the right hind leg and abdominal area of 6-week-old BALB/c female mice under pentobarbital anesthesia were unhaired using a hair clipper and depilatory cream. A 5×5 mm range of the epidermis located below the knee joint of the unhaired hind leg was scarified with ten bundled 27G intradermal needles. Then, 1×10⁶ PFU/10 μl herpes simplex virus type 1, HSV-1 (strain 7401) was dropped and coated thereon for infection. Allodynia and hyperalgesic reactions of the hind legs were measured using each of 6 von Frey hairs having different strengths (0.03, 0.17, 0.41, 0.69, 1.20, and 1.48 g). Mice were housed in acryl cages (110×180×150 mm), and then habituated in this environment for 15 minutes. Then, von Frey hairs were applied vertically to the plantar of the hind leg, such that it bended slightly, for 3 to 5 seconds. No significant change was observed in reactivity against the filament until the night on day 4 after inoculation. Reactivity started to increase from the morning on day 5 after inoculation, and it continuously increased even on day 7 after inoculation. The development of allodynia and hyperalgesia were confirmed on the hind legs on the infection side.

### Example 3

### Production of Neuropathic Pain Model—3 (Post-Herpetic Neuralgia Model)

Post-herpetic neuralgia models were produced according to the method described in JP Patent Application No. 2001-136736. Specifically, the right hind leg and abdominal area of 6-week-old BALB/c female mice under pentobarbital anesthesia were unhaired using a hair clipper and depilatory cream. A 5×5 mm range of the epidermis located below the knee joint of the unhaired hind leg was scarified with ten bundled 27G intradermal needles. Then, 1×10⁶ PFU/10 μl herpes simplex virus type 1, HSV-1 (strain 7401) was dropped and coated thereon for infection. HSV-1-infected mice were subjected to treatment with an antiviral agent acyclovir (acyclovir was orally administered every 3 hours, 5 times a day, for 7 days from day 5 after infection) to cure the skin lesions. Then, allodynia reaction and hyperalgesic reaction were measured in a manner similar to Example 2 after the cure of the skin lesions. By the treatment with acyclovir, 92% of the mouse individuals infected with HSV-1 survived. Increased reactivity against the filament was observed and thereby the development of allodynia and hyperalgesia was confirmed in approximately 65% of these mice. In addition, allodynia and hyperalgesia continued until 40 days after the administration of the virus.

### Example 4

### Extraction of mRNA from the Spinal Cord of the Pain Model, and Identification of a Gene Showing Enhanced Expression in the Spinal Cord Where Pain Symptoms were Exhibited

Individuals among the Chung models and the shingles pain models exhibiting the symptoms of allodynia and hyperalgesia were dissected after decapitation, and a region on the side exhibiting the symptoms of allodynia and hyperalgesia and a region on the opposite side of the myeloid tissues (L4-6) were excised separately. Further, in the case of post-herpetic neuralgia models, individuals exhibiting the symptoms of allodynia and hyperalgesia and individuals exhibiting no such symptoms were dissected after decapitation. The spinal cords (L4-6) were excised respectively from these individuals. Immediately after excision, these spinal cords were frozen with liquid nitrogen, and then stored for a while at −80° C. until preparation of mRNA. Using as starter materials the spinal cord region on the side exhibiting the symptoms of allodynia and hyperalgesia and that on the opposite side, and the spinal cords of the individuals exhibiting the symptoms of allodynia and hyperalgesia and the individuals exhibiting no such symptoms, fragments containing genes showing enhanced expression in the spinal cords of the side and the individuals exhibiting pain symptoms were collected according to the attached protocols using PCR-select cDNA subtraction kit (Clontech; hereinafter, abbreviated as “a subtraction kit”). Specifically, total RNA was prepared from each spinal cords using ISOGEN (Nippon Gene) according to its attached protocols. Then, mRNA was obtained using Dynabeads Oligo (dT)₂₅ and a magnet Dynal MPC-S (Dynal) according to its attached protocols. After double-stranded cDNA was synthesized using Time Saver™ cDNA synthesis kit (Amersham Pharmacia), cDNA was digested with a restriction enzyme Rsa I, and then ligated to Adaptors 1 and 2 within the subtraction kit. Subsequent procedures including hybridization, PCR amplification, and TA cloning of amplified cDNA fragments, preparation of transformants, recovery of plasmids from transformed Escherichia coli, and the like were performed using a subtraction kit and other generally known kits according to these attached protocols. The determination of nucleotide sequences of the thus obtained cDNA fragments was performed for 136 cDNA clones of the Chung model, and 288 cDNA clones each of the shingles pain model and of the post-herpetic neuralgia model. After the determination of the nucleotide sequences, homology search was performed by BLAST in the official database, GenBank Database. Thus, 6 cDNA clones whose sequences had not been published, were selected, and were respectively named ch7L-7, ch7L-14, ch8L-32, phM-8, phS-3, and phX-6.

### Example 5

### Confirmation of Fluctuations in Gene Expression by Dot-Blot Hybridization

For the genes identified by the subtraction method, whether or not these genes actually differed in their expression levels between samples was examined using a dot-blot hybridization method. These genes had been TA-cloned as cDNA fragments, and inserted in vectors as described above. Using the recombinant vector as a template, cDNA fragments were amplified by the PCR method. Specifically, per PCR reaction, 2.5 μl of a PCR buffer, 1.5 μl of 25 mM MgCl₂, 2.5 μl of 2 mM dNTP mix, 0.5 μl of AmpliTaq Gold, 0.5 μl of 30 pmol/pl primer, 0.5 μl of a template cDNA clone, and 16.5 μl of sterilized water were mixed. The mixed solution was incubated at 95° C. for 10 minutes, and then a cycle of 95° C. for 30 seconds, 55° C. for 30 seconds, and 72° C. for 30 seconds was repeated 30 times. The concentration of the thus obtained PCR product was measured using Pico Green (Molecular Probe). Then the product was diluted at a given concentration to be an alkaline solution. Next, membranes wherein 40 or 60 ng each of the solution was dot-blotted on Hybond-N⁺ at an interval of 1 cm were prepared. After drying the membrane, DNA was immobilized to the membrane by UV cross-linking.

Among the Chung models, individuals exhibiting the symptoms of allodynia and hyperalgesia were dissected after decapitation, and a region on the side exhibiting the symptoms of allodynia and hyperalgesia and a region on the opposite side of the spinal cords (L4-6) were excised separately. Total RNA solutions were obtained from each of the tissues using a method similar to Example 4. To remove the genomic DNA, 3 μl of 10× DNase I buffer, 3 μl of DNase I (1 unit/μl) and DEPC treatment water were added to 3 μg of the RNA to 30 μl. After incubation for 15 minutes at room temperature, 3 μl of 25 mM EDTA was added to the solution, and then incubated at 65° C. for 15 minutes to stop reaction. Next, RNA subjected to the removal of the genome was diluted to 200 ng/μl, incubated at 95° C. for 5 minutes, allowed to stand in ice for 5 minutes for denaturation, and then allowed to react with an alkaline phosphatase labeling reagent, thereby preparing a probe.

Hybridization was performed on the membrane, on which the PCR product had been blotted, with the RNA probes, which had been prepared from the spinal cord region on the side exhibiting the symptoms of allodynia and hyperalgesia and that on the opposite side. Then, signals of chemoluminescence were detected on X-ray film using AlkPhos Direct. X-ray film images were incorporated into Image Master, and then the spot image analysis was performed by microarray analysis software, Array Gauge ver. 1.2. Image analysis revealed that at least ch7L-7 and ch8L-32 genes increased in the spinal cord on the side exhibiting the symptoms of allodynia and hyperalgesia 1.5 fold or more than that on the opposite side.

### Example 6

### Construction of Standard cDNA Library

cDNA library was constructed according to Methods Enzymol., 303; 19, 1999, and JP Patent Publication (Unexamined Application) No. 2000-325080. Specific examples are as follows.

Preparation of mRNA

0.5 to 1 g of sample of each organ or tissue (kidney (adult), lungs (adult), stomach (adult), tongue (adult), ES cells, liver (13 days embryo and 10 days embryo) of mice (C57BL/6) was homogenized in 10 ml of a suspension, and then a mixed solution of 1 ml of 2 M sodium acetate (pH 4.0) and an equivalent volume of phenol/chloroform (volume ratio 5:1) was added to the suspension for extraction. After extraction, an equivalent volume of isopropanol was added to the aqueous layer, so that RNA was separated and precipitated from the aqueous layer. The samples were incubated on ice for 1 hour, and then subjected to a cooled centrifuge for 15 minutes at 4000 rpm, and the precipitate was recovered. The specimen, i.e., the precipitate, was washed with 70% ethanol, and then dissolved in 8 ml of water. Next, 16 ml of an aqueous solution (pH 7.0) containing 2 ml of 5 M NaCl, and 1 CTAB (cetyltrimethyl ammoniumbromide), 4 M urea, and 50 mM Tris was added to the solution, so that RNA was precipitated and polysaccharides were removed (CTAB precipitation method). Subsequently, the solution was subjected to a centrifuge at 4000 rpm for 15 minutes at room temperature and RNA was recovered, and then the RNA was dissolved in 4 ml of 7 M guanidine-Cl. After a 2-fold volume of ethanol was added to the solution, the solution was incubated on ice for 1 hour, and then subjected to a centrifuge at 4000 rpm for 15 minutes. The resulting precipitate was washed with 70% ethanol, thereby recovering RNA. The RNA was dissolved in water again, and then the purity of RNA was measured by reading OD ratio 260/280 (>1.8) and 230/260 (<0.45).

Preparation of First Strand cDNA

Reverse transcription reaction was conducted using 15 μg of mRNA and 3000 units of Superscript II (Gibco-BRL) in a reaction solution in a final volume of 165 μl under the presence of 0.54 mM each of 5-methyl-dCTP, dATP, dTTP, and dGTP, 0.6 M trehalose, 50 mM Tris-HCl (pH 8.3), 75 mM KCl, 3 mM MgCl₂, 10 mM DTT, 52 ng/μl BSA, and 5 units of RNase inhibitor. 12.6 μl of oligonucleotide containing the recognition sequence of Xho I (1st primers: 1st NX primers and 1st BS primers) was used as primers. 1st NX primers were used for constructions of libraries derived from kidney (adult) and lungs (adult); 1st BS primers were used for constructions of libraries derived from stomach (adult), tongue (adult), ES cells, and liver (13 days embryo and 10 days embryo). The sequences of the 1st primers are as follows:

1st NX primers: 5′-GAGAGAGAGAGCGGCCGCAACTCGAG(T)16VN-3′ (SEQ ID NO: 23; V=A, G or C; N=A, G, C or T; restriction sites, Not I and Xho I), and

1st BS primers: 5′-GAGAGAGAGAAGGATCCAAGAGCTC(T)16VN-3′ (SEQ ID NO: 24; V=A, G or C; N=A, G, C or T; restriction sites, Bam HI and Sst I).

Upon the start of the reaction, ¼ aliquot of the reaction solution was removed, and 1.5 μl of [a-³²P]-dGTP (3000 Ci/mmol, 10 μCi/μl) was added to the aliquot, so that the synthesis efficiency of the 1st strand cDNA was measured. 0.5 μl of the RI-labeled reaction solution was spotted on DE-81 paper, and then the RI activity was measured and calculated before and after washing three times with 0.5 M sodium phosphate buffer (pH 7.0). Thereafter, the RI-labeled reaction solution and the unlabeled reaction solution were mixed. 8 μl of 0.5 M EDTA, 2 μl of 10% SDS, and 20 μg of proteinase K were added to the mixed solution, and then the solution was heated at 45° C. for 15 minutes. After phenol/chloroform extraction and ethanol precipitation, the precipitate was dissolved in 47 μl of water treated to be RNase-free (hereinafter, referred to as RNase-free water).

Biotinylation of RNA diol

To bind biotin to the diol sites of RNA, which are present at both the 5′ terminus having Cap structure, and ribose of the 3′ terminus having poly A chain, two-step reaction was conducted. In other words, the two-step reaction contains oxidation of diol groups and the subsequent coupling reaction between biotinhydrazide and oxidized RNA. First, 15 μg of RNA-1st strand cDNA complexes obtained in reverse transcription reaction was treated in 50 μl of a reaction solution with 6.6 mM sodium acetate buffer (pH 4.5) and sodium periodate as an oxidant. The oxidation reaction was performed under a shading condition on ice for 45 minutes. Subsequently, 11 μl of 5 M sodium chloride, 0.5 μl of 10% SDS, and an equivalent volume of isopropanol were added to the solution. Then the solution was allowed to stand on ice for 60 minutes, and centrifuged at 4° C. for 15 minutes at 15000 rpm for precipitation. The precipitate was washed with 70% ethanol, and then re-dissolved in 50 μl of RNase-free water. 5 μl of 1 M sodium acetate (pH 6.1), 5 μl of 10% SDS, and 150 μl of 10 mM biotin hydrazide were added to the sample, and then allowed to react overnight at room temperature (22 to 26° C.). Finally, 5 μl of 5 M NaCl, 75 μl of 1M sodium acetate (pH 6.1), and a 2.5 fold volume of ethanol were added to the solution. The solution was cooled on ice for 1 hour, and then centrifuged at 4° C. for 15 minutes, so that biotinylated RNA-DNA complexes were re-precipitated. The precipitate was washed once with 70% ethanol, washed once with 80% ethanol, and then dissolved in 70 μl of RNase-free water.

Selection of Full-Length cDNA Using RNase I

mRNA for which elongation of complete cDNA could not be obtained at the reverse transcription reaction, and biotin residues which had been labeled at the 3′ terminus of mRNA, were removed by treating with RNase I that digests single-stranded RNA. Specifically, 10 μl of 10× RNase I buffer (100 mM Tris-HCl (pH 7.5), 50 mM EDTA, 2 M NaOAc), and 200 units of RNase I were added to 70 μl of the sample subjected to the biotinylation reaction, and single-stranded RNA was digested at 37° C. for 15 minutes.

Collection of Full-Length cDNA

To prevent non-specific adsorption of cDNA to streptavidin-coated magnetic beads, 5 mg (500 μl) of the magnetic beads were pre-treated with 100 μg of yeast tRNA (pre-treated with DNase I). The beads were allowed to stand on ice for 1 hour, and then washed with 50 mM EDTA and 2 M NaCl solution. The beads were suspended in 500 μl of 50 mM EDTA and 2 M NaCl solution, and then cDNA treated with RNase I was added to the suspension. Full-length cDNA were bound to magnetic beads by stirring the suspension at room temperature for 30 minutes. Beads capturing full-length cDNA were washed 4 times with 50 mM EDTA and 2 M NaCl solution, once with solution containing 0.4% SDS and 50 μg/μl yeast tRNA, once with solution containing 19 mM NaCl, 0.2 mM EDTA, 10 mM Tris-HCl (pH 7.5), and 20% glycerol, once with 50 μg/μl yeast tRNA aqueous solution, and once with RNaseH buffer (20 mM Tris-HCl (pH 7.5), 10 mM MgCl₂, 20 mM KCl, 0.1 mM EDTA, and 0.1 mM DTT). After suspension in 100 μl of RNaseH buffer, 3 units of RNase H was added to the suspension, and then the suspension was heated at 37° C. for 30 minutes. Subsequently, 1 μl of 10% SDS and 2 μl of 0.5 M EDTA were added to the suspension, and then the suspension was exposed at 65° C. for 10 minutes, thereby collecting the supernatant. The thus collected single-stranded full-length cDNA was extracted with phenol/chloroform, and after the amount of liquid was reduced to 100 μl or less using Speed Bag, the cDNA solution was subjected to G25/G100 Sephadex chromatography. While fractions having RI activity were collected in a microtube treated with silicon, 2 μg of glycogen was added to the fraction. The precipitate was obtained from the fraction by ethanol precipitation and dissolved in 30 μl of extra pure water.

Addition of oligo dG to the first strand cDNA

30 μl of 1st strand cDNA collected above was subjected to oligo dG addition reaction for 30 minutes at 37° C. in a reaction solution with a final volume of 50 μl under the presence of 200 mM sodium cacodylate (pH 6.9), 1 mM MgCl₂, 1 mM CoCl₂, 1 mM 2-mercaptoethanol, and 100 μM dGTP, using 32 units of terminal deoxynucleotidyl transferase. At the end of the reaction, EDTA was added to 50 mM. After a series of phenol/chloroform extraction and ethanol precipitation, the precipitate was dissolved in 31 Pl of extra pure water.

Synthesis of Second Strand cDNA

2nd strand cDNA was synthesized using the 1st strand cDNA as a template as follows. In a reaction system with a final volume of 60 μl, 3 μl of the second strand low buffer [200 mM Tris-HCl (pH 8.75), 100 mM KCl, 100 mM (NH₄)₂SO₄, 20 mM MgSO₄, 1% Triton X-100, and 1 mg/μl BSA], 3 μl of the second strand high buffer [200 mM Tris-HCl (pH 9.2), 600 mM KCl, and 20 mM MgCl₂], 0.25 mM each of dCTP, dATP, dTTP and dGTP, 6 μl of β-NADH, 31 μl of the 1st strand cDNA with an oligo dG, and 600 ng of the 2nd strand primer-adapter were added. As the 2nd strand primer-adapter, 2nd AT was used for constructions of libraries derived from kidney (adult) and lungs (adult), 2nd X was used for constructions of libraries derived from stomach (adult), tongue (adult), ES cells, and liver (13 days embryo and 10 days embryo). Using the reaction system, the 2nd strand cDNA was synthesized using 15 units of ExTaq DNA polymerase, 150 units of thermostable DNA ligase, and 3 units of thermostable RNase H. Then, 1 μl of 0.5 M EDTA was added to the reaction system to stop reaction. To further denature protein components, the cDNA was heated at 45° C. for 15 minutes in the presence of 1 μl of 10% SDS and 10 μg of proteinase K. Finally, after phenol/chloroform extraction and ethanol precipitation, the thus purified double-stranded full-length cDNA was obtained. The sequences of the 2nd primer-adapters are as follows:

2nd AT: 5′-GAGAGAGAGAAGGATCCAAGAGCTCAATTAATTAATTAAACCCC CCCCCCC-3′ (SEQ ID NO: 25; restriction sites, Bam HI and Sst I), and

2nd X: 5′-GAGAGAGAGATTCTCGAGTTAATTAAATTAATCCCCCCCCCCCCC-3′ (SEQ ID NO: 26; restriction sites, Xho I and C13).

The double-stranded full-length cDNA obtained by the above methods w as inserted into λZAPIII vector, and then collected into libraries. λZAPIII vect or was prepared by changing a partial sequence of the multi-cloning site of λZ AP II (STRATAGENE) vector, AAAAGCTGGAGCTCCACCGCGGTGGCGGCCG CTCTAGAACTAGTGGATCCCCCGGGCTGCAGGAATTCGATATCAAGCTTATCGA TACCGTCGACCTCGA (SEQ ID NO: 27), with AAAAGCTGGAGCTATGGCCCT TATGGCCGAGCTCGCGGCCGCGAATTCCTCGAGGGCCGATTTGGCCAATCGAG (SEQ ID NO: 28), and to newly introduce two Sfi I sites.

### Example 7

### Construction of Normalization/Subtraction cDNA Libraries

In construction of standard cDNA libraries, normalization/subtraction was not performed for construction of libraries, and λZAP III was used as a vector. In this example, a similar method to the example of constructing standard cDNA libraries, except using λPS (RIKEN) a vector, in addition to λAPIII, and performing normalization/subtraction for construction of libraries, was employed. λPS (RIKEN) [named as λ-FLC-1; “FLC” means full-length cDNA.] is a XPS vector altered to be adapted for cDNA. That is, Bam HI and Sal I, which are convenient for insertion of cDNA, were respectively introduced into cloning sites existing at both ends of 10 kbp stuffer, and a 6 kb DNA fragment was inserted to Xba I site to allow cloning of approximately 0.5 kb to 13 kb cDNAs (see FIG. 1 of JP Patent Publication (Unexamined Application) No. 2000-325080). When λ-FLC-1 was used, for example, in the case of cDNA libraries derived from lungs, the mean chain length of the inserts was 2.57 kb. Specifically, 0.5 kb to 12 kb of inserts could be cloned. In the case of using conventional λZAP, the mean chain length of inserts was 0.97 kb, suggesting that the use of λ-FLC-1 enables more efficient cloning of cDNA even in a large size compared to λZAP.

Normalization/subtraction of cDNA libraries are described hereunder. Preparation of driver: mRNA that had been used as a starter material [(a) RNA driver] and RNA that had been prepared by in vitro transcription reaction were used as drivers. The latter RNA can be classified into two types: (b) and (c) RNA drivers. For preparation of one RNA driver, cDNA was collected from RNA-cDNA that had been removed by normalization, and then cloned into a phage vector. After infection of Escherichia coli with it, 1000 to 2000 plaques per starter material were picked up and mixed to construct one library (mini library). The phages within the library were then converted into a plasmid DNA by a standard method. That is, Escherichia coli was re-infected with the phage together with helper phages to produce a phagemid, and then Escherichia coli was infected again with the phagemid to produce plasmid DNA. For the thus obtained DNA, in vitro transcription reaction was conducted using T3 RNA polymerase or T7 RNA polymerase. After treatment with DNase I and proteinase K, phenol/chloroform extraction was performed, and thereby RNA [(b) RNA driver] was obtained. At this preparation, 9 types of mini libraries were prepared respectively from tissues of pancreas, liver, lungs, kidney, brain, spleen, testis, small intestine, and stomach, and then the mini libraries were mixed together and used for obtaining RNA.

For preparation of the other RNA driver, a library consisting of stored clones from which redundancies had been previously removed (including approximately 20,000 clones) were cultured. For DNA prepared from the cultured library, in vitro transcription reaction was performed similarly to the preparation (b) RNA driver, and thereby the (c) RNA driver was obtained. These 3 types of RNAs were biotinylated to label using Label-IT Biotin Labeling kit, and then added to a tester cDNA at a ratio of 1:1:1. Reaction with Rot 10 was performed at 42° C., and then the supernatant collected after treatment with streptavidin beads (CPG). For the supernatant, 2nd strand DNA was synthesized. Synthesis of the tester cDNA and that of the 2nd strand DNA were performed according to the method described for preparation of standard cDNA libraries in Example 6.

### Example 8

### Determination of the Nucleotide Sequence of cDNA and Registration of the Sequence Into Database

Determination of the nucleotide sequence of cDNA and registration of the sequence into database were performed according to the methods described in Nature, 409; 685, 2001. An example is as described below. cDNA that had been synthesized using normalization/subtraction as described in Example 7 was cloned into λZAP, and sequences at the 3′ termini of cDNAs, which was cloned in the obtained transformants, were sequenced. Clustering was performed by comparing these sequences, clusters having unpublished sequences were selected, and then one representative clone was selected from each cluster. The representative clones were selected by Q-bot (Genetix Limited), and then prepared for an array on a 384-well plate. At this time, Escherichia coli was cultured in 50 μl of LB medium (100 μg/ml ampicillin and 50 μg/ml kanamycin, or 100 μg/ml ampicillin and 25 μg/ml streptomycin were respectively added for PS/DH10B and ZAP/SOLR host/vector systems) at 30° C. for 18 to 24 hours.

For the purpose of extracting plasmids and determining the sizes of cDNAs inserted in the plasmids, each clone was cultured in 1.3 ml of HT solution containing 100 μg/ml ampicillin, and then plasmid DNAs were isolated and purified from the culture using QIAprep 96 Turbo (QIAGEN). 1/30 aliquot of the purified plasmid DNA was digested with Pvu II, and then subjected to 1% agarose gel electrophoresis, so that the size of cDNA inserted in the plasmid was determined.

3 types of sequencers were used to determine the full-length nucleotide sequence of cDNA. Depending on the length of the inserted sequence, cDNAs were classified into two categories: cDNAs shorter than 2.5 kb, and cDNAs of, or longer than, 2.5 kb. The short clones were sequenced from both ends. In this determination, Licor DNA4200 (long read sequencer) and a Thermosequenase Primer Cycle Sequencing kit were used. The long clones were sequenced by the shotgun method. In this sequencing, Shimadzu RISA 384 and a DYEnamic ET Terminator Cycle Sequencing kit were used. To prepare a shotgun library, 48 PCR-amplified DNA fragments from 48 independent representative clones were used. The identification of these representative clones were previously confirmed by end sequencing, pooled and concatenated, and then subjected to a shearing step using the Double Stroke Shearing Device (Fiore Inc.). The termini of these DNA fragments were truncated using T4 DNA polymerase to make blunt-ends. The DNA fragments were cloned into pUC18, and then further transformed into DH10B. Shotgun sequencing was performed with 12 to 15 redundancies. The remaining gaps were filled up by primer walking as described above.

As described above, the nucleotide sequences were determined, and then the nucleotide sequences, for which the quality had been checked, were registered in an in-house database.

### Example 9

### In-House Database Search and Characterization of Gene

Against sequence information in the above in-house database, homology search was performed using BLAST for 6 types of polynucleotides obtained in Example 4, ch7L-7, ch7L-14, ch8L-32, phM-8, phS-3, and phX-6. As a result, clone ID NO: C630002H18 in the database was hit for ch7L-7, clone ID NO: C130099K08 in the database was hit for ch7L-14, clone ID NO: 5930404010 in the database was hit for ch8L-32, clone ID NO: A330018G02 in the database was hit for phM-8, clone ID NO: 9930124N15 in the database was hit for phS-3, and clone ID NO: B330016G20 in the database was hit for phX-6. Consequently, sequence information described respectively in SEQ ID NOS: 1 to 6 of the present sequence listing was obtained. The nucleotide sequence of ch7L-7 corresponds to nucleotide Nos. 2159 to 2276 of SEQ ID NO: 1. The nucleotide sequence of ch7L-14 corresponds to nucleotide Nos. 2349 to 2530 of SEQ ID NO: 2. The nucleotide sequence of ch8L-32 corresponds to nucleotide Nos. 2888 to 3052 of SEQ ID NO: 3. The nucleotide sequence of phM-8 corresponds to nucleotide Nos. 1897 to 2329 of SEQ ID NO: 4. The nucleotide sequence of phS-3 corresponds to nucleotide Nos. 1059 to 1302 of SEQ ID NO: 5. The nucleotide sequence of phX-6 corresponds to nucleotide Nos. 1474 to 1871 of SEQ ID NO: 6. Clones of the cluster groups respectively represented by the obtained clones are also homologous each other, and therefore all the nucleotide sequences corresponding to the clone IDs of the cluster groups can be considered to be within the scope of the invention as the nucleotide sequences of the present invention. The candidate amino acid sequences of the gene products according to the present invention, SEQ ID NOS: 12 to 17, were deduced from the nucleotide sequences of SEQ ID NOS: 1 to 6. The protein motif search was performed against protein motif database Pfam.

In ch7L-7 gene product, Leucine rich Repeat (LRR) and Leucine rich Repeat C-terminal domain were present. LRR is thought to be involved in protein-protein interaction and cell adhesion. Since ch7L-7 gene product having amino acid sequence represented by SEQ ID NO: 12 has approximately 50% homology with a leucine-rich glioma-inactivated 1 protein (Oncogene, 17; 2873, 1998, Acta Neuropathol., 103; 255, 2002) having high homology with a protein involved in development, differentiation, and maintenance of the nerve system, it is thought to have an important role in nerves.

In ch7L-14 gene product, Protein Phosphatase 2C-like sequence was present. Since ch7L-14 gene product having amino acid sequence represented by SEQ ID NO: 13 has high homology with CaM-kinase phosphatase, which is thought to inactivate CaM-kinase that reacts with calcium, which is extremely important to cellular function (J Biochem. (Tokyo), 129; 193, 2001). Since CaM-kinase phosphatase is widely distributed in the nerve system including the spinal cord, it is thought to have an important role in regulation of pain in the spinal cord.

In ch8L-32 gene product, TMS membrane protein/tumor differentially expressed protein (TDE) was present. Since ch8L-32 gene product having amino acid sequence represented by SEQ ID NO: 14 has extremely high homology with membrane protein TMS-1 (J Exp Biol., 203; 447, 2000), which contains glutamic acid involved in many brain and nerve functions including pain, and is rich in nerves, it is though to have an important role in regulation of pain in the nerve system.

In phM-8 gene product, Zinc finger, C2H2 type (ZnF_C2H2) was present. ZnF_C2H2 has a protein structure having nucleic acid binding ability and takes part in transcription regulatory functions. Since mouse homologue (msal) of Drosophila spalt gene (MechDev., 56; 117, 1996), and Zep (J Biochem. (Tokyo), 124; 1220, 1998), which are strongly expressed in the spinal cord, also have ZnF_C2H2, phM-8 gene product having amino acid sequence represented by SEQ ID NO: 15 is thought to take part in transcriptional regulation of some proteins in the spinal cord.

In phX-6 gene product, emp24/gp25L/p24-like sequence was present. A protein family whose members have emp24/gp25L/p24-like sequence is known to have a carrier function involved in intracellular protein transport, particularly in transport from the endoplasmic reticulum, and to be present in membranes.

As described above, motif analysis and other characterizations for the novel genes of the present invention strongly suggested that the genes of the present invention which show increased expression in the spinal cord of a model animal developing allodynia and hyperalgesia are genes relating to pain.

### Example 10

### Human Genome Database Search

To screen for homologues of ch7L-7, ch7L-14, ch8L-32, phM-8, phS-3, and phX-6 from the human genome database, the homologous sequences of the nucleotide sequences thereof were searched by accessing the public genome data base and using a program called Spidey. This program is available at http://www.ncbi.nlm.nih.gov/IEB/Research/Ostell/Spidey/.

Open reading frames of the homologous sequences were predicted based on the search result, and human homologues of ch7L-7, ch7L-14, ch8L-32, phS-3, and phX-6 were obtained. These nucleotide sequences are respectively set forth in SEQ ID NOS: 7 to 11. Further, amino acid sequences were predicted from the polynucleotide sequences described as SEQ ID NOS: 7 to 11. The predicted amino acid sequences are respectively set forth in SEQ ID NOS: 18 to 22.

According to the present invention, there are provided polynucleotides having nucleotide sequences composing novel genes, and the gene products thereof; polypeptides, antibodies and the like, which are applicable to biology, medicine, veterinary medicine and the like. These molecules can be utilized in a reagent or a pharmaceutical for diagnosing or assessing, or preventing or treating pain including, but are not limited to, e.g., neuropathic pain, shingles pain, and post-herpetic neuralgia; or for screening for a compound or the like which acts on the molecule of the present invention.

The invention is not to be limited in scope by the specific embodiments illustrated in the specification, and functionally equivalent methods and components are within the scope of the invention. Indeed various modifications of the invention, in addition to those shown and described herein will become apparent to those skilled in the art from foregoing description. Such modifications are intended to fall within the scope of the appended claims.

All references cited herein, including patent applications, patents, and other publications, are incorporated by reference herein in their entireties for all purposes.

What is claimed is:

1. An isolated polynucleotide, comprising a nucleotide sequence selected from the group consisting of SEQ ID NOS: 1, 2, 4, 7, 8, 9, 10 and 11.

2. An isolated polynucleotide, which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, wherein said isolated polynucleotide comprises a nucleotide sequence encoding a polypeptide relating to pain.

3. An isolated polynucleotide, comprising a nucleotide sequence having at least 70% homology with the nucleotide sequence selected from the group consisting of SEQ ID NOS: 1, 2, 4, 7, 8, 9, 10 and 11, and encoding a polypeptide relating to pain.

4. An isolated polynucleotide, comprising a nucleotide sequence encoding a polypeptide consisting of an amino acid sequence selected from the group consisting of SEQ ID NOS: 12, 13, 15, 18, 19, 20, 21 and 22.

5. A recombinant vector, comprising the polynucleotide of claim 2.

6. A recombinant expression vector, comprising the polynucleotide of claim 2 functionally related to a nucleotide regulatory element that regulates the expression of a nucleotide sequence in a host cell.

7. A genetically engineered host cell, comprising the polynucleotide of claim 2.

8. A genetically engineered host cell, comprising the polynucleotide of claim 2 functionally related to a nucleotide regulatory element that regulates the expression of a nucleotide sequence in a host cell.

9. An isolated polypeptide, which is encoded by the polynucleotide of claim 2.

10. An isolated polypeptide, comprising the amino acid sequence selected from the group consisting of SEQ ID NOS: 12, 13, 15, 18, 19, 20, 21 and 22.

11. An isolated polypeptide, which has at least 70% homology with the amino acid sequence selected from the group consisting of SEQ ID NOS: 12, 13, 15, 18, 19, 20, 21, and 22, wherein said isolated polypeptide relates to pain.

12. A method for producing a polypeptide, comprising culturing a transformant transformed with a recombinant vector comprising the polynucleotide of claim 2 to produce a polypeptide encoded by the polynucleotide.

13. An antibody, which is raised against the polypeptide of claim 9.

14. A transgenic animal, containing the polynucleotide of claim 2.

15. The transgenic animal of claim 14, wherein said polynucleotide is an expression transgene incorporated into the genome of the animal.

16. The transgenic animal of claim 14, in which the expression of said polynucleotide is blocked or suppressed, or promoted.

17. A method for screening for a compound that suppresses or enhances an expression of a polynucleotide, wherein said polynucleotide hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

18. A kit for screening for a compound that suppresses or enhances an expression of a polynucleotide, wherein said polynucleotide hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

19. The method of claim 17, wherein said method uses a genetically engineered host cell that contains the polynucleotide which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

20. The method of claim 17, wherein said method uses a transgenic animal that contains the polynucleotide which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

21. A method for screening for a compound that acts on the polypeptide of claim 9, which uses said polypeptide.

22. The method of claim 21, wherein said action is the promotion or inhibition of the activity of the polypeptide.

23. A method for screening for a compound that acts on the antibody of claim 13, which uses said antibody.

24. The method of claim 23, wherein said action is the promotion or inhibition of the activity of said antibody.

25. A kit for screening for a compound that acts on a polypeptide by using the polypeptide, wherein said polypeptide is encoded by the polynucleotide which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

26. A pharmaceutical composition, comprising the polypeptide of claim 9.

27. A pharmaceutical composition, comprising the antibody of claim 13.

28. A method for treating or preventing pain, comprising administering the pharmaceutical composition of claim 26 to a subject.

29. A method for treating or preventing pain, comprising administering the pharmaceutical composition of claim 27 to a subject.

30. The method of claim 28, wherein said pain is neuropathic pain.

31. The method of claim 29, wherein said pain is neuropathic pain.

32. The method of claim 30, wherein said neuropathic pain is shingles pain or post-herpetic neuralgia.

33. The method of claim 31, wherein said neuropathic pain is shingles pain or post-herpetic neuralgia.

34. A method for diagnosing a pain patient for the risk of having a disease accompanied by pain, or the degree of pain, comprising measuring the differential expression of the polypeptide of claim 9 in the sample obtained from said pain patient.

35. A method for monitoring the therapeutic effect by a pharmaceutical composition in a pain patient, comprising measuring the differential expression of the polypeptide of claim 9 in a sample obtained from said pain patient, wherein said pharmaceutical composition contains the polypeptide encoded by the polynucleotide which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

36. A method for monitoring the therapeutic effect by a pharmaceutical composition in a pain patient, comprising measuring the differential expression of the polypeptide of claim 9 in a sample obtained from said pain patient, wherein the pharmaceutical composition contains an antibody against a polypeptide encoded by the polynucleotide which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

37. A method for diagnosing a pain patient for the risk of having a disease accompanied by pain, or the degree of pain, comprising measuring the differential expression of the polynucleotide of claim 2 in a sample obtained from said pain patient.

38. A method for monitoring the therapeutic effect by a pharmaceutical composition in a pain patient, comprising measuring the differential expression of the polynucleotide of claim 2 in a sample obtained from said pain patient, wherein the pharmaceutical composition contains a polypeptide encoded by the polynucleotide which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

39. A method for monitoring the therapeutic effect by a pharmaceutical composition in a pain patient, comprising measuring the differential expression of the polynucleotide of claim 2 in a sample obtained from said pain patient, wherein the pharmaceutical composition contains an antibody against a polypeptide encoded by the polynucleotide which hybridizes under stringent conditions to the polynucleotide consisting of the nucleotide sequence selected from the group consisting of SEQ ID NOS: 1, 2, 4, 7, 8, 9, 10 and 11, or a polynucleotide consisting of a nucleotide sequence complementary to the polynucleotide, and comprises a nucleotide sequence encoding a polypeptide relating to pain.

40. The method of claim 34, wherein said pain patient is a patient with neuropathic pain.

41. The method of claim 35, wherein said pain patient is a patient with neuropathic pain.

42. The method of claim 37, wherein said pain patient is a patient with neuropathic pain.

43. The method of claim 38, wherein said pain patient is a patient with neuropathic pain.

44. The method of claim 40, wherein said patient with neuropathic pain is a patient with shingles pain or post-herpetic neuralgia.

45. The method of claim 41, wherein said patient with neuropathic pain is a patient with shingles pain or post-herpetic neuralgia.

46. The method of claim 42, wherein said patient with neuropathic pain is a patient with shingles pain or post-herpetic neuralgia.

47. The method of claim 43, wherein said patient with neuropathic pain is a patient with shingles pain or post-herpetic neuralgia.

48. A method for diagnosing pain, which uses the antibody of claim 13.
